(19) United States
(12) Patent Application Publication
(10) Pub. No.: US 2013/0035479 A1
(43) Pub. Date: Feb. 7, 2013

(54) FUNCTIONALIZED BETA 1,6 GLUCOSAMINE DISACCHARIDES AND PROCESS FOR THEIR PREPARATION

(71) Applicant: OM PHARMA, Meyrin 2 (CH)

(72) Inventors: Stephane MOUTEL, Bonne (FR); Jacques BAUER, Saint-Prex (CH); Carlo CHIAVAROLI, Thoiry (FR)

(73) Assignee: OM PHARMA, Meyrin 2 (CH)

(21) Appl. No.: 13/632,838

(22) Filed: Oct. 1, 2012

Related U.S. Application Data
(63) Continuation of application No. 12/514,882, filed on Sep. 1, 2009.
Application No. 12/514,882 is a continuation-in-part of application No. PCT/IB2006/003244, filed on Nov. 16, 2006.

(51) Int. Cl. C07H 1/00; C07H 1/02

(52) U.S. Cl. 536/17.1; 536/55.2; 536/17.2; 536/17.6; 536/17.9

(57) ABSTRACT

The present invention relates to a novel process for the chemical synthesis of β-(1->6)-linked glucosamine disaccharides of the formula (1) and (intermediate) compounds relating to the process. According to further aspects the invention relates to compositions comprising the compounds and the use of the compounds in the synthesis of disaccharides and medicine.

### FIELD OF THE INVENTION

The present invention relates to a novel process for the chemical synthesis of β-(1→6)-linked glucosamine disaccharides. Such compounds may be used as lipid A derivatives. An example of a lipid A derivative is OM-174-DP®, first isolated by OM PHARMA,¹ from partially degraded Escherichia coli Lipopolysaccharides. This invention includes the design and chemical synthesis of new lipid A analogs which have lost both sugar-O-acyl substituents (at O-3 and O-3′) and therefore carry only the N-linked fatty acid residues. The immunological activities of such compounds is related to that of the parent biological OM-174-DP®.

### BACKGROUND OF THE INVENTION

Lipopolysaccharides (LPS) are the major compounds expressed at the outer membrane of almost all Gram-negative bacteria. These amphiphilic macromolecules possess a common structure composed of a hydrophilic polysaccharide (formed from a core oligosaccharide and an O-specific polysaccharide) covalently linked to a lipophilic moiety called lipid A,² which serves as the LPS membrane anchor.

LPS, also known as endotoxins, are potent stimulators of host defense systems, both as adjuvants for vaccine antigens³ and as inducers of non specific resistance to infection in animal models.⁴ These amphiphilic macromolecules possess extremely potent immunostimulating activities.⁵ The biological activity of LPS is due principally to the lipid A constituent while the toxicity of lipid A is strictly dependant on its primary structure.

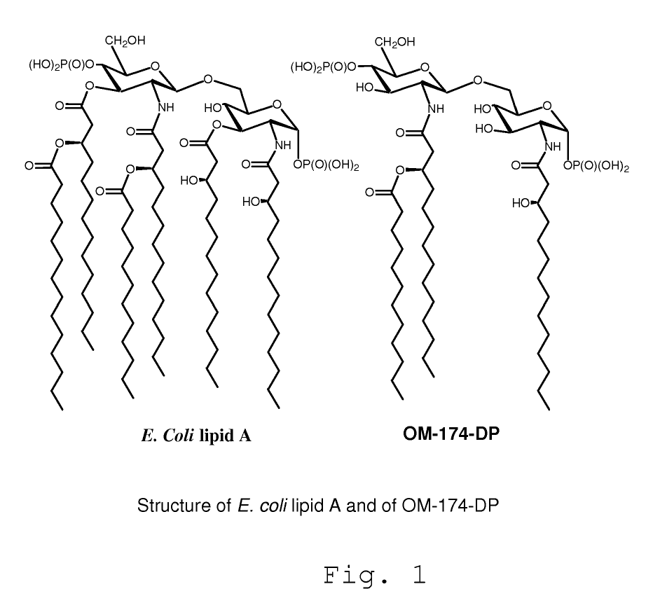

Generally, lipid A has a highly conservative structure. It is generally composed of a β-(1→6)-linked glucosamine disaccharide backbone, phosphorylated at positions O-1 and O-4′ and six or more fatty acyl groups linked as esters and amides. The configuration of the anomeric phosphate (O-1 position) of the reducing glucosamine part is α without exception. For example, the complete chemical structure of the lipid A isolated from E. coli cells (FIG. 1), elucidated by Imoto et al⁶ contains a β-(1→6)-linked glucosamine disaccharide backbone, phosphorylated at positions O-1 and O-4′ and acylated at 2, 3 position with (R)-3-hydroxytetradecanoic acid, at 2′ position with (R)-3-dodecanoyloxytetradecanoic acid and at 3′ position with (R)-3-tetradecanoyloxytetradecanoic acid.

Enormous interest from both industrials and academic research laboratories arose due to the broad spectrum of biological activities. Much effort has been dedicated to chemical modifications of the lipid A structures with the goal of reducing the natural endotoxicity of the parent compound while maintaining or improving its beneficial immunostimulating properties. In the 1980s, Ribi et al studied a chemical process with the intention to uncoupling the toxic effects of natural Salmonella Chlamydia RC595 lipid A from potentially useful immunomodulatory effects. This process, based on a selective hydrolysis of 1-phosphono group⁷ and (R)-3-hydroxytetradecanoyl⁸ residue attached to the 3-position of lipid A sugar, furnished the known monophosphoryl lipid A (MPL®) immunostimulant which is an effective adjuvant in prophylactic and therapeutic vaccines⁹ with a greatly reduced toxicity compared to its parent lipid A. However, MPL® as well as naturally derived lipid A is a mixture of several components due to the inherent heterogeneity of the LPS and incomplete chemoselective hydrolysis steps or purification steps. Consequently, MPL® immunostimulant comprises several less highly acylated compounds in addition to the major hexaacyl compound.

In the early 90's, a new lipid A derivative (OM-174-DP®, FIG. 1) was isolated by OM PHARMA from partially degraded E. coli LPS.¹ This derivative has lost both sugar-O-acyl substituents (at O-3 and O-3′) and therefore carries only the N-linked fatty acid residues of E. coli lipid A, namely a (R)-3-hydroxytetradecanoyl group at N-2 and a (R)-3-dodecanoyloxytetradecanoyl group at N-2′, thus leaving only three long-chain acyl groups on the structure. Thorough pharmacological investigations of this new compound revealed that it has potent antitumor activity in several in vivo tumor models¹⁰ and that it is an effective immunoadjuvant with very low toxicity.

Structure-activity relationship of lipid A was extensively studied over the last two decades. Shiba and co-workers have directed numerous efforts towards the study of structure-activity relationship of synthetic E. coli lipid A and efforts to develop the chemical synthesis of such compounds. They have first realized the chemical synthesis of the monophosphoryl E. coli lipid A¹¹ but especially they have unequivocally confirmed the structure of E. coli lipid A by total chemical synthesis based on N-Troc protected glucosamine derivates.¹² Many structural variations of E. coli lipid A have been reported by the same group in terms of the acyl moieties (types, numbers and location on the sugar backbone)¹³ and in terms of glycosyl phosphate moiety (phosphonoxyethyl analog with α or β configuration at position 1).¹⁴

In 1997, they have described the most efficient synthesis of a precursor of lipid A.¹⁵ By this route, several unnatural analogs have been reported with modifications of the acyl chains¹⁶ and modifications of the glycosyl phosphate moiety and synthesis of lipid A itself.¹⁷ The group has published the chemical synthesis of lipid A isolated from Helicobacter pylori using the improved route¹⁸. Their publication includes a triacylated lipid A analog lacking both sugar-O-acyl substituents (at O-3 and O-3′). However, in addition to this the compound also lacks a substitution at the 4′-O position.

Shiba's work was a source of inspiration for later syntheses of various lipid A. For evidence, synthetic Chlamydia tetra- and pentaacyl lipid A analogs have been recently synthesized by Kosma and co-workers in order to clarify the role of lipid A in Chlamydia associated infections.¹⁹ Biomira group has developed unnatural synthetic lipid A structure containing novel lipid moieties mimicking the naturally occurring E. coli derived and Salmonella derived lipid A structures.²⁰ The chemical synthesis of P. gingivalis lipid A, a triacylated lipid A carrying only the N-linked fatty acid residues and lacking the 4′-O-phosphate group was also reported by Ogawa and co-workers.²¹

LPS and its related compounds have mainly been investigated as LPS-agonists. In recent years, lipid A related compounds have been studied as LPS-antagonists, which may have potential as immunosuppressants, and in autoimmune diseases and septicemia by deactivating LPS-induced aggressive macrophages. For example, Qureshi and co-workers²² have isolated a non toxic lipid A as a potent LPS antagonist from Rhodobacter sphaeroides (Rs-DPLA) and an Eisai group has developed the total synthesis of the proposed structure with their own methodology²³ and a related compound namely E5564a potent anti-septicemia drug.²⁴ Existing lipid A synthetic methodologies previously described based on a final hydrogenolysis¹¹⁻²¹ could not be applicable due to an olefinic functionality present in the proposed Rs-DPLA. In recent years, related compounds of Rs-DPLA and E5564 were synthesized²⁵.

Lipopolysaccharides and lipid-A molecules are immunostimulating agents because they activate toll like receptor 4 (TLR4), even though some LPS however may activate TLR2, such as LPS from Porphyromonas gingivalis²⁶. Normally, TLR2 responses are only induced by agents such as muramyl peptides (MPM), bacterial lipopeptides (BLP), peptidoglycans (PGN) and lipoteichoic acids (LTA). Very interestingly, the inventors of the present invention have now found that the synthetic compounds of the invention (and not only OM-174-DP derived from natural sources, as already described in a poster²⁷ or a recent review²⁸) are preferentially acting via human TLR2, and not, as it is the case in murine cells, preferentially via the expected TLR4 route. This interspecies remarkable property (preferential TLR4 in murine cells and rather TLR2 in human cells) has not been disclosed previously.

### SUMMARY OF THE INVENTION

The prior art discussed above does not disclose synthetic lipid A analogs lacking both sugar-O-acyl substituents (at O-3 and O-3′) and comprising a 4′-O-phosphate group or an alternative substitution at the 4′-O position. Such Lipid A analogs have beneficial properties and have utility in the field of (human) medicine. However, these lipid A analogs can only be obtained laboriously from natural sources e.g. by specific hydrolysis processes. In addition, obtaining these compounds from natural sources in a pharmaceutically acceptable purity is a further technological challenge, especially because the raw materials in general are obtained from potentially pathogenic organisms. In view of these problems it is the aim of the present invention to provide such compounds in synthetic form. For this the present invention according to a first aspect provides a novel process for the chemical synthesis of β-(1→6)-linked glucosamine disaccharides.

A further aspect of the invention relates to a process suitable for treating products obtained with the synthesis process of the invention. The products treated with this treatment process have an altered physico-chemical constitution and according to a preferred embodiment have an increased biological activity.

According to still further aspects the present invention relates to the compounds obtainable with the processes of the invention, intermediate compounds of the synthesis process, compositions comprising these compounds and the use of these compounds in an organic synthesis process and/or medicine.

It is worth to be mentioned here that the compounds of the invention are preferentially acting via human TLR2.

### DETAILED DESCRIPTION OF THE INVENTION

An important step in the process according to the invention is the glycosylation reaction between a compound of the formula 10:

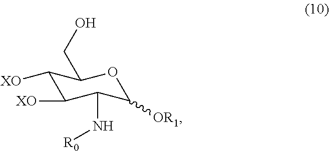

wherein:
R₁ is a group selected from a (C₃-C₆) alkenyl, such as a C₃ or C₄ alkenyl, preferably 2-propenyl or 1-propenyl;
X is a hydrogen, a group selected from benzyl or a substituted benzyl, such as 4-methoxybenzyl or 3,4-dimethoxybenzyl or 2,5-dimethoxybenzyl or 2,3,4-trimethoxybenzyl or 3,4,5-trimethoxybenzyl;
R₀ is selected from R₅ or R₂, wherein R₅ is selected from:
(i) an acyl group derived from a, straight chain-carboxylic acid having from 2 to 24 carbon atoms, preferably a hydroxy acyl group, such as a 3-hydroxy acyl group, an oxo acyl group such as a 3-oxo acyl group, an amino acyl group such as a 3-amino acyl group;
(ii) an acyloxyacyl group, preferably a 3-acyloxyacyl group, an acylaminoacyl group, preferably a 3-acylaminoacyl group, an acyl thioacyl group, preferably a 3-acylthioacyl group;
(iii) an alkyloxyacyl group, preferably a (C₂-C₂₄) alkyloxyacyl group, an alkenyloxyacyl group, preferably a (C₂-C₂₄) alkenyloxyacyl group, an alkynyloxyacyl group, preferably a (C₂-C₂₄) alkynyloxyacyl group an alkyl aminoacyl group, preferably a (C₂-C₂₄) alkylaminoacyl group, an alkenylaminoacyl group, preferably a (C₂-C₂₄) alkenylaminoacyl group, an alkynylaminoacyl group, preferably a (C₂-C₂₄) alkynylaminoacyl group, an alkylthioacyl group, preferably a (C₂-C₂₄) alkylthioacyl group, an alkenylthioacyl group, preferably a (C₂-C₂₄) alkenylthioacyl group, an alkynylthioacyl group, preferably a (C₂-C₂₄) alkynylthioacyl group, an acyl group derived from a branched chain-carboxylic acid having from 2 to 48 carbon atoms, preferably a carboxylic acid branched at the 3-position;
   wherein in the groups (i), (ii), (iii) the hydrocarbon chain of the acyl may be saturated or unsaturated and the hydrocarbon chain of the acyl, alkyl, alkenyl, alkynyl may be branched or straight and optionally may be substituted with one or more groups independently selected from halogen such as fluoro, chloro, bromo, or iodo; a hydroxyl or hydroxyl derivative —OY, wherein Y is as defined below; an amine or amine derivative —NHW, wherein W is as defined below; a group —OZ,
   wherein Z is selected from (f), (g), (h), (i), (j), (k) as defined below;
and R₂ is a group selected from a (C₁-C₆) halogenated alkoxy carbonyl, such as 2,2,2-trichloroethoxycarbonyl (TROC) or a 1,1-dimethyl-2,2,2-trichloroethoxycarbonyl (TCBOC);
   with a compound of the formula 7:

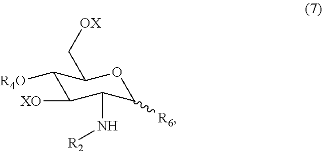

wherein R₄ is selected from:
(a) an acyl group as defined in (i), (ii) or (iii) for R₅;
(b) a branched or straight alkyl group, preferably a branched or straight (C₁-C₂₄) alkyl group; a branched or straight alkenyl group, preferably a branched or straight (C₁-C₂₄) alkenyl group; a branched or straight alkynyl group, preferably a a branched or straight (C₁-C₂₄) alkynyl group;
(c) a group —[(C₁-C₂₄) alkyl]-COOX, —[(C₂-C₂₄) alkenyl]-COOX or —[(C₂-C₂₄) alkynyl]-COOX wherein X is as defined below
(d) a group —[(C₁-C₂₄) alkyl]-NHW, —[(C₁-C₂₄) alkenyl]-NHW or —[(C₁-C₂₄) alkynyl]-NHW wherein W is as defined below;
(e) a formyl alkyl group, preferably a formyl [(C₁-C₂₄) alkyl] group; a formyl alkenyl group, preferably a formyl [(C₁-C₂₄) alkenyl] group; a formyl alkynyl group, preferably a formyl [(C₁-C₂₄) alkynyl] group;
(f) a dimethoxyphosphoryl group;
(g) a group —P(O)(OY)₂, wherein Y is as defined below;
(h) a group —P(O)(OH)—O[(C₁-C₂₄) alkyl]-NHW, —P(O)(OH)—O[(C₁-C₂₄) alkenyl]-NHW or —P(O)(OH)—O[(C₁-C₂₄) alkynyl]-NHW wherein W is as defined below;
(i) a group —P(O)(OH)—O[(C₁-C₂₄)alkyl], —P(O)(OH)—O[(C₁-C₂₄) alkenyl], or —P(O)(OH)—O[(C₁-C₂₄)alkynyl];
(j) a group —P(O)(OH)—O[(C₁-C₂₄) alkyl]-COOX, —P(O)(OH)—O[(C₁-C₂₄) alkenyl]-COOX, —P(O)(OH)—O[(C₁-C₂₄) alkynyl]-COOX, wherein X is as defined above;
(k) a group —S(O)(OH)₂;
(l) a protective group selected from benzyl or a substituted benzyl, such as 4-methoxybenzyl or 3,4-dimethoxybenzyl or 2,5-dimethoxybenzyl or 2,3,4-trimethoxybenzyl or 3,4,5-trimethoxybenzyl; or from a (C₃-C₆) alkenyl, such as a C₃ or C₄ alkenyl, preferably 2-propenyl or 1-propenyl;
wherein alkyl, alkenyl, alkynyl groups may be branched or straight and may be unsubstituted or optionally are substituted with one or more groups independently selected from halogen such as fluoro, chloro, bromo, or iodo; a hydroxyl or hydroxyl derivative —OY, wherein Y is as defined below; an amine or amine derivative —NHW,
wherein W is as defined below; or a group —OZ, wherein Z is selected from (f), (g), (h), (i), (j), (k);
and wherein Y is selected from hydrogen; an (C₃-C₆) alkenyl, such as a C₂ or C₃ alkenyl, preferably 2-propenyl or 1-propenyl group; a group selected from benzyl or a substituted benzyl, such as 4-methoxybenzyl or 3,4-dimethoxybenzyl or 2,5-dimethoxybenzyl or 2,3,4-trimethoxybenzyl or 3,4,5-trimethoxybenzyl; a O-Xylylene group;
and wherein W is selected from hydrogen; a benzyloxycarbonyl group or a 9-fluorenylmethyloxycarbonyl;
   and wherein R₆ is a group selected from trichloroacetimidate, fluoride, chloride, bromide, and X and R₂ are as defined above.

The reaction may be carried out according to a general method for glycosylation known in the art, such as the method described in Angew. Chem., Int. Ed. Engl., (1986), 212. This method uses dichloromethane as a solvent and a catalytic amount of acid such as trimethylsilyltrifluoromethanesulfonate. When using this method only the β-disaccharide according to formula 11h is obtained.

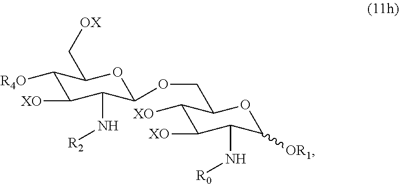

wherein R₁, R₂, R₄, R₀ and X are as defined above. A bond as the one connecting OR₁ indicates that both the α and β anomer are possible.

R₅ may be selected from an acyl group as defined in (i) or alternatively a branched acyl group as defined in (ii), (iii). The acyl group, may be selected from the group comprising an acyloxyacyl group, an acylaminoacyl group, an acylthioacyl group, a (C₁-C₂₄) alkyloxyacyl group, a (C₁-C₂₄) alkylaminoacyl group, and a (C₁-C₂₄) alkylthioacyl group. (Cn-Cn), wherein n is an integer, such as (C₁-C₂₄) and (C₂-C₂₄) as used in this specification means that the saturated or unsaturated hydrocarbon chain it refers to may contain the number of carbon atoms indicated in the interval such as 1 to 24 carbon atoms and 2 to 24 carbon atoms respectively, such as 2, 4, 6, 8, 10, 12, 14, 16, 18, 20, 22 carbon atoms. Acyl, alkyl, alkenyl and alkynyl hydrocarbon chains in the acyl and acyl derivatives defined in (i), (ii) or (iii) may each individually comprise from 1 to 50 carbon atoms such as from 2 to 48 carbon atoms, including 1 to 24 carbon atoms, such as from 2 to 24 carbon atoms, in particular 2, 4, 6, 8, 10, 12, 14, 16, 18, 20, 22 carbon atoms. As such in a (C₂-C₂₄)alkyloxyacyl group for example the alkyl hydrocarbon may comprise from 2 to 24 carbon atoms and the hydrocarbon chain of the acyl moiety may comprise from 2 to 24 carbon atoms.

The hydrocarbon chain of the acyl groups may be saturated or may comprise one or more unsaturated carbon double or triple bonds. In addition to this hydrocarbon chains of acyl, alkyl, alkenyl and alkynyl may be branched or straight and may optionally be substituted with one or more groups independently selected from halogen such as fluoro, chloro, bromo, or iodo; a hydroxyl or hydroxyl derivative —OY, wherein Y is as defined before; an amine or amine derivative —NHW, wherein W is as defined before; a group —OZ, wherein Z is selected from (f), (g), (h), (i), (j), (k) as defined before.

In the case of the acyloxyacyl group, two acyl groups are linked via an oxygen atom, in the case of the acylaminoacyl group via an NH group, and in the case of the acylthioacyl group via a sulphur atom. The (C₁-C₂₄) alkyloxyacyl group, the (C₁-C₂₄) alkylaminoacyl group and the (C₁-C₂₄) alkylthioacyl group may be obtained starting from the corresponding hydroxy fatty acid.

Acyl groups are preferably substituted at the 3-position, such as a 3-acyloxyacyl group, a 3-acylaminoacyl group, and the 3-acylthioacyl group. The same applies to the aforementioned (C₁-C₂₄) alkyl equivalents.

Preferably the members of the group R₅ comprise one or two acyl moieties, preferably selected from fatty acid residues, hydroxy fatty acid residues and oxy fatty acid residues. When the acyloxyacyl group is a 3-acyloxyacyl group, these acyl moieties preferably comprise a 3-hydroxy fatty acid residue or for the ester-linked group a 3-oxo fatty acid residue. Typical examples of the acyloxyacyl group are 3-hydroxy (C₄-C₂₄)-fatty acid-acyls which are ester-linked at the 3-hydroxy position with a (C₁-C₂₄)-carboxylic acid. Preferably the acyloxyacyl group is a 3-hydroxy (C₈-C₁₈)-fatty acid-acyl which is ester-linked at the 3-hydroxy position with (C₁₀-C₁₈)-fatty acid. Such acyloxyacyl groups are present in the lipid A component of Gram-negative bacteria, such as Escherichia coli, Haemophilus influenzae, Campylobacter jejuni, Rhodocyclus gelatinosus, Chromobacterium violaceum, Neisseria meningitides, Salmonella minnesota.

In a first group of preferred glucosamine disaccharides according to the invention the acyloxyacyl group selected for R₅ is the 3-hydroxy C₁₄-fatty acid-acyl ester-linked at the 3-hydroxy position with the C₁₂-fatty acid, with this acyloxyacyl group at the N2′-position. In another preferred glucosamine disaccharide according to the invention the acyloxyacyl group selected for R₅ is the 3-hydroxy C₁₄-fatty acid-acyl ester-linked at the 3-hydroxy position with the C₁₄-fatty acid, and the acyloxyacyl group is preferably at the N-2′ position.

In another preferred glucosamine disaccharide according to the invention the acyloxyacyl group selected for R₅ is the 3-hydroxy C₁₄-fatty acid-acyl ester-linked at the 3-hydroxy position with the C₁₂-fatty acid, with this acyloxyacyl group at the N-2 position. In another preferred glucosamine disaccharide according to the invention the acyloxyacyl group selected for R₅ is the 3-hydroxy C₁₄-fatty acid-acyl ester-linked at the 3-hydroxy position with the C₁₂-fatty acid, with the acyloxyacyl group at both the N2-position and the N-2′-position.

When a compound of the invention comprise a chiral centre the inventions encompasses all R- and S enantiomers, and any racemic mixture.

The other selection for R₅ may be an acyl group or also an acyloxyacyl group. According to a second group of disaccharides according to the invention the acyl group is a 3-hydroxy (C₄-C₂₄)-fatty acid, preferably a 3-hydroxy (C₁₀-C₁₈)-fatty acid. The 3-hydroxy group of such a fatty acid may be protected with a group X as defined previously. In the preferred disaccharides according to the invention the acyl group is a 3-hydroxy C₁₄-fatty acid, at the N2-position or at the N2′-position.

However, the R₅ may also be an acyloxyacyl group defined hereinbefore, and comprising an 3-hydroxy (C₄-C₂₄)-fatty acid-acyl which is ester-linked at the 3-hydroxy position with (C₁-C₂₀)-carboxylic acid, preferably an 3-hydroxy (C₈-C₁₈)-fatty acid-acyl ester-linked at the 3-hydroxy position with (C₁₀-C₁₈)-fatty acid. More preferred is the disaccharide wherein R₅ at the N2 position is the 3-hydroxy C₁₄-fatty acid-acyl ester-linked at the 3-hydroxy position with the C₁₂-fatty acid or C₁₆-fatty acid, and wherein R₅ at the N2′ position is the 3-hydroxy C₁₄-fatty-acid-acyl ester-linked at the 3-hydroxy position with the C₁₂-fatty acid or C₁₄-fatty acid.

According to a preferred embodiment a first group R₅ is selected from the subgroup (i) as defined and a second group R₅ is selected from a subgroup (ii) or (iii) as defined in claim 1, wherein preferably the group R₅ at the N-2 position is selected from (i). In alternative embodiments the groups R₅ are both selected identically or differently from the subgroup (i) or are both selected identically or differently from a subgroup (ii) or (iii).

It is noted, that in the group R₅ the acyl groups and/or the acyl and alkyl group may be interlinked.

In this specification the term “fatty acid residue” means: a substantially hydrophobic chain of C₂-C₃₀ atoms, which chain may be straight, branched, saturated, mono- or polyunsaturated, having inserted one or more hetero atoms such as nitrogen, oxygen, sulphur, and which chain may be substituted with one or more substituents, such as hydroxyl, oxo, acyloxy, alkoxy, amino, nitro, cyano, halogeno, sulphydryl, provided that the biological activity is not substantially adversely affected. An example of a substituted fatty acid residue (comprising an amide-linked substituent) is disclosed by Onozuka, K. et al. in Int. J. Immunopharmac, Volume 15, pages 657-664 [1993]).

R₄ may be selected from (a)-(l) as defined above. The alkyl, alkenyl, alkynyl chains in these substituents for R₄ may be branched or straight and may be unsubstituted or optionally are substituted with one or more groups independently selected from halogen such as fluoro, chloro, bromo, or iodo; a hydroxyl or hydroxyl derivative —OY, wherein Y is as defined defore; an amine or amine derivative —NHW, wherein W is as defined before. For the groups (a), (b), (c), (d), (e) the optional substituents may furthermore comprise a group —OZ, wherein Z is selected from (f), (g), (h), (i), (j), (k). Preferably R₄ is selected from (f), (g), (h), (i) or (j), more preferably from (g). Preferably the groups (a), (b), (c), (d), (e), (f), (g), (h), (i), (j), comprise from 1 to 50 carbon atoms, such as from 2 to 24 carbon atoms.

In a subsequent step a number of the (C1-C6) halogenated alkoxy carbonyl protective groups R₂ are hydrolytically removed from the compound of formula 11h. In this specification a number of shall mean one or more unless otherwise specified. It is preferred that all groups R₂ of the compound of formula 11h are removed. If R₀ is selected as R₅ then the compound of formula 11h will comprise a single group R₂. If R₀ is selected as R₂ then the compound of formula 11h will comprise two groups R₂ and it will be preferred to remove both these groups. The groups R₂ may be removed with any suitable means know to the skilled person. It is know to the skilled person that (C1-C6) halogenated alkoxy carbonyl protective groups such as Troc may be removed using zinc-copper couple in acetic acid and water.

If R₀ is selected as R₅ then a compound of the formula 12a will be obtained.

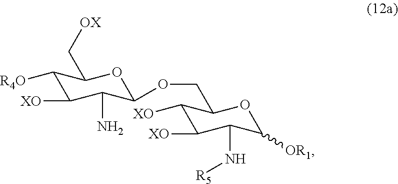

wherein R1, R4, R5 and X are as defined before. If R₀ is selected as R₂ in formula 11h, then a compound of the formula 12b will preferably be obtained:

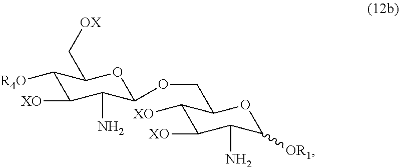

wherein R1, R4, and X are as defined before.

To the free amino group of the compound of formula 12a or 12b a group R₅ is attached. This may be accomplished by reacting a compound of formula 12a or 12b with a (activated) carboxylic acid corresponding to said group R₅. The reaction may be performed in any way know to the skilled person such as by using a coupling agent such as isobutyl chloroformate or 1-isobutyloxy 2-isobutyloxycarbonyl-1,2-dihydroquinoleine or a carbodiimide. In the reaction of the compound 12a the (activated) carboxylic acid corresponding to said group R₅ may comprise a group R₅ identical or different from the group R₅ of the compound of formula 12a.

The reaction of the compound of formula 12a or 12b with a (activated) carboxylic acid corresponding to said group R₅ results in the formation of a compound of the formula 13:

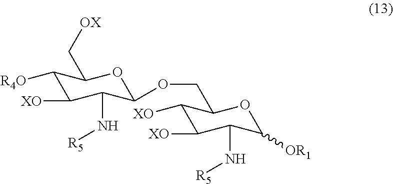

wherein R₁, R₄, R₅, and X are as defined previously. The groups R₅ may be identical or different. Whether the groups R₅ of compound 13 are identical or different may depend on the fact whether compound 12a or compound 12b is used in the reaction and the nature of the (activated) carboxylic acid used in the reaction. If compound 12b is used it is possible to select the group R₅ of the (activated) carboxylic acid different from the group R₅ of the compound 12b. In that case the groups R₅ of compound 13 will differ. However, the group R₅ of the (activated) carboxylic acid may also be identical to the group R₅ of compound 12b. And it will be clear that in that case the groups R₅ of compound 13 will be identical. If compound 12a is reacted with a single (activated) carboxylic acid the groups the groups R₅ of compound 13 will be identical. However, it is also possible to use combinatorial chemistry and to react compound 12b with a number of differing (activated) carboxylic acids. In that case a mixture of compounds according to the general formula 13 will be obtained in which the groups R₅ are identical or different. The skilled person will understand that the number of different compounds of the general formula 13 and their ratios in the mixture will depend on the number of differing (activated) carboxylic acids used in the reaction and their ratios. It is preferred that at least one of R₅ is selected from a branched acyl group as defined in (ii), (iii). More preferably the group R₅ connected to the N₂′-position is selected as a branched acyl group.

A hemiacetal of the formula 14:

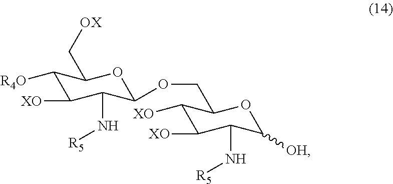

wherein R₄, R₅, and X are as defined above, is formed by removal of the group R₁ from the compound of the formula 13. The deprotection of a (C₃-C₆) alkenyl group may be achieved in any way known to the skilled person. For example an (C₃-C₆) alkenyl group may be removed in a two-step conversion. If the (C₃-C₆) alkenyl group is for example 2-propenyl first, the allyl group in 13 may be isomerized into 1-propenyl by treatment with hydrogen-activated Iridium catalyst such as commercially available ([bis(methyldiphenylphosphine)]-(1,5-cyclooctadiene)Iridium(I) hexafluorophosphate) in a polar solvent such as tetrahydrofuran (Synthesis, (1981), 305-308). The 1-propenyl group may then be cleaved with an aqueous iodine source such as iodine or N-Bromosuccinimide. (J. Chem Soc., Chem. Commun., (1982), 1274). Different selections of the group R₁ may be removed in analogy.

Compound 13 and the hemiacetal of the formula 14 are important intermediates in the synthesis process according to the invention. Depending on the reactions performed on compounds 13 and 14 and the intermediates derived there from form a great number of different protected β-(1→6)-linked glucosamine disaccharides with different substitutions R₈ on the O-1 position may be obtained. These protected β-(1→6)-linked glucosamine disaccharides may be represented with the general formula 15:

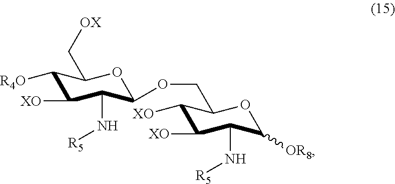

wherein R₄, R₅, and X are as defined previously and R₈ is selected from (a), (b), (c), (d), (e), (f), (g), (h), (i), (j) or (k) as defined previously for R₄.

In one embodiment of the synthesis process of the invention the free hydroxyl group of compound 14 may be phosphorylated in any way known to the skilled person. For this commercially available tetrabenzyl pyrophosphate may be used in the presence of a suitable base in a polar solvent. The base may be selected from lithium bis(trimethylsilyl)amide and the solvent may be selected from tetrahydrofuran. Phosphorylation of compound 14 results in a compound of the formula 15a:

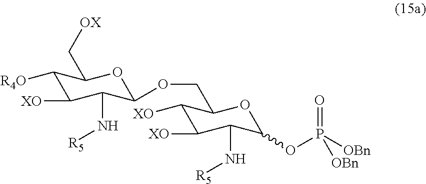

Phosphorylation may be of use to obtain compounds having substitutions at the O-1 position selected from (g), (h), (i) or (j) as defined for R₄. If necessary the phosphate group obtained in compound 15a may be further derivatized.

In a different embodiment the free hydroxyl group of compound 14 may be sulphated in any way known to the skilled person. Sulfatation of compound 14 results in a compound of the formula 15b:

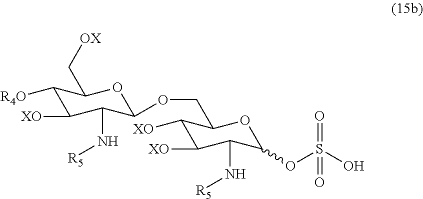

In yet a different embodiment the process according to the invention further comprises reacting the free hydroxyl group of compound 14 with an (activated) carboxylic acid of the formula R₈OH, wherein R₈ is selected from (a) as defined previously for R₄. The reaction may take place in any way known to the skilled person such as in the presence of a coupling agent such as isobutyl chloroformate or 1-isobutyloxy 2-isobutyloxycarbonyl-1,2-dihydroquinoleine or a carbodiimide under formation of a compound of the formula 15c:

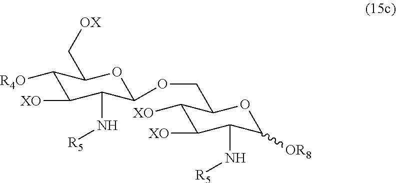

wherein R₄, R₅, and X are as defined before, and R₈ is selected from (a) as defined previously for R₄ and wherein R₈ may be in the α or β configuration and preferably is in the β configuration.

In yet a different embodiment a group that may function in a subsequent reaction as a leaving group, such as a trichloroacetimidate group, is coupled to the free hydroxyl group of compound 14. This may be effected in any way known to the skilled person e.g. by reacting compound 14 with trichloacetonitrile in the presence of a mineral base such as cesium carbonate or potassium carbonate in a polar solvent, preferably an aprotic polar solvent such as dichloromethane. This reaction of compound 14 results in a compound of the formula 24:

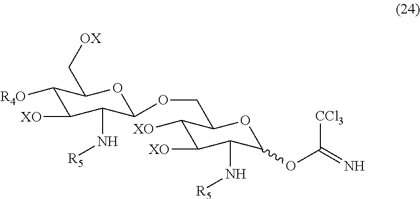

Compound 24 may be reacted further with an organic molecule R₈OH to replace the trichloroacetimidate group with the group R₈. R₈ may be selected from (b), (c), (d), (e) as defined for R₄.

The reaction of the acetimidate group with an organic alcohol is known to the skilled person. It may take place in a polar solvent, preferably an aprotic polar solvent such as dichloromethane in the presence of a catalytic amount of acid such as trimethylsilyltrifluoromethanesulfonate and may be performed in analogy with the method described in Angew. Chem., Int. Ed. Engl., (1986), 212. The reaction of compound 24 with the compound R₈ results in a compound of the formula 15d:

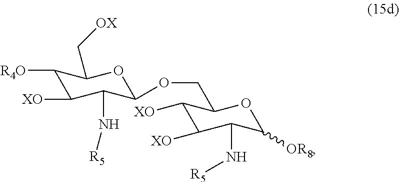

Wherein R₄, R₅, R₈ and X are as defined above and wherein R₈ may be in the α or β configuration and preferably is in the β configuration.

Compounds 13, 15a, 15b, 15c and 15d may be reacted further such as to remove any protecting groups selected form X, Y, W other then from H. Removal of protecting groups may be accomplished according to methods known in the art. Benzyl protecting groups may for example be removed by hydrogenolysis in the presence of a high-grade metal such as palladium on carbon. Allyl groups and analogous groups may be removed as discussed above for the removal of the allyl group from compound 13. Removal of 4-methoxybenzyl or 3,4-dimethoxybenzyl or 2,5-dimethoxybenzyl or 2,3,4-trimethoxybenzyl or 3,4,5-trimethoxybenzy or phenyl or 4-methoxyphenyl or 3,4-dimethoxyphenyl or 2,5-dimethoxyphenyl or 2,3,4-trimethoxyphenyl or 3,4,5-trimethoxyphenyl groups may be accomplished by oxidative cleavage such as with dichlorodicyanoquinone (DDQ) or Ceric ammonium nitrate (CAN). An O-Xylylene group and a benzyloxycarbonyl group may be removed by hydrogenolysis in the presence of a high-grade metal such as palladium on carbon. A 9-fluorenylmethyloxycarbonyl may be removed by a base such as piperidine, morpholine. It will be understood that different protecting groups may be removed independently. Therefore, any protecting group present within R₈ could be removed prior to removal of X.

Reactive groups initially present on R₈ or after removal of a protective group may be reacted further before removal of (additional) protective groups. If R₈ comprises a number of free hydroxyl groups, esters, including phosphate and sulfate esters, and ethers may be formed with methods known in the art. Free hydroxyl groups may furthermore be oxidized with known methods to obtain a carboxylic acid or a ketone. If R₈ comprises a number of carboxylic acid groups, esters or amide may be formed with methods known in the art. If R₈ comprises a number of free amine groups an amide may be formed with methods known in the art. If R₈ comprises a number of unsaturated carbon bonds these may be reacted with osmium tetra oxide with methods known in the art to obtain a α, β hydroxylated group. The free hydroxyl groups of such a α, β hydroxylated group may be reacted further before removal of protecting groups.

In addition to this the phosphate group may be methylated with methods known in the art, such as by reaction with CH₂N₂. It should be noted that such methylation with CH₂N₂ may take place before or after removal of protective groups on the β-(1→6)-linked glucosamine disaccharides including a protective group selected from X, as defined above.

In an alternative embodiment of the process of the invention the protective groups of compound 14 are removed with methods know in the art, such as those described above.

In a further alternative embodiment of the process of the invention the unsaturated bond of the (C3-C6) alkenyl group of compound 13, such as a C3 or C2 alkenyl, preferably 2-propenyl or 1-propenyl is hydrogenated to the corresponding alkyl.

In yet a further alternative embodiment of the process of the invention the (C3-C6) alkenyl group of compound 13, is selected as 2-propenyl and the unsaturated bond of the 2-propenyl group is reacted with osmium tetra oxide with methods known in the art to obtain a α, β hydroxylated group. The free hydroxyl groups of such a α, β hydroxylated group may be reacted further before removal of protecting groups. It will be clear that with the synthesis process according to the invention a great number of β-(1→6)-linked glucosamine disaccharides according to the formula 1 may be obtained:

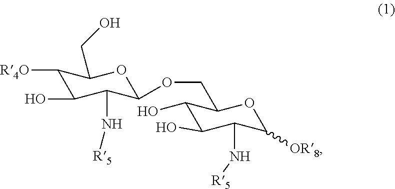

wherein R₄′, R₅′ and R₈′ are as defined previously for R₄, R₅ and R₈ respectively, wherein any Y or W are H, and wherein the selection of R₈′ furthermore includes H.

Compound 7 which is involved in the process according to the invention may be obtained by coupling a leaving group selected from trichloroacetimidate, fluoride, chloride, bromide, to the free hydroxyl group of a compound of formula 6:

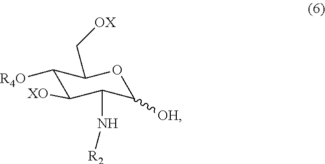

wherein R₂, R₄ and X are as defined previously. This may be accomplished by any suitable method known in the art. For example treatment of the compound of the formula 6 with trichloroacetonitrile, preferably in the presence of a base, more preferably a mineral base, such as cesium carbonate or potassium carbonate, in a polar solvent, preferably an aprotic polar solvent such as dichloromethane. Protection with chlorine and bromine may be accomplished by reaction with acetic anhydride in a solvent such as pyridine and subsequent reaction with gaseous HCl or HBr in acetic acid respectively. Protection with fluorine may be accomplished by reaction with acetic anhydride and subsequent reaction with diacyl amino sulfur trifluoride (DAST).

The compound of formula 6, may be obtained by removing with known methods the group R₁ from the compound of the formula 5:

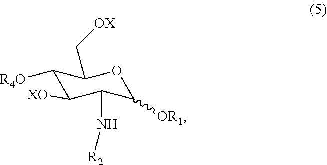

wherein R₁, R₂, R₄ and X are as defined previously. For example the deprotection of an allyl group may be achieved in two-step conversion. First, the allyl group may be isomerized into 1-propenyl by treatment with hydrogen-activated Iridium catalyst such as commercially available ([bis(methyldiphenylphosphine)]-(1,5-cyclooctadiene)Iridium(I) hexafluorophosphate) in a polar solvent such as tetrahydrofuran according to a method described in Synthesis, (1981), 305-308. The propenyl group may then be cleaved with aqueous iodine source such as iodine or N-Bromosuccinimide. (J. Chem Soc., Chem. Commun., (1982), 1274).

The compound of formula 5 may be obtained in a number of different reactions depending on the selection of the group R₄. These reactions may start from the compound of the formula 4:

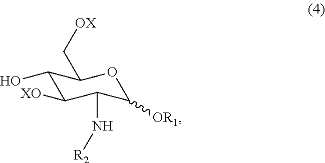

wherein R₁, R₂ and X are as defined previously. Starting from compound 4a number of different substituents may be added as R₄ to the free hydroxyl group of this compound. These substituents may be added with general methods known in the art.

If R₄ is selected from (f), (g), (h) (i) or (j) the process according to the invention may comprise phosphorylation under suitable reaction conditions of the free hydroxyl group of the compound of the formula 4:

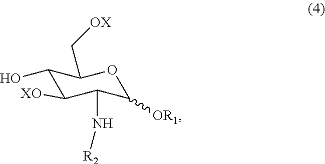

wherein R₁, R₂, and X are as defined before. This may be accomplished for example by reaction with a phosphoramidite reagent, such as a diaryl N,N dialkyl phosphoramidite or a diallyl N,N dialkyl phosphoramidite, preferably diallyl N,N diisopropyl phosphoramidite, in the presence of a coupling agent, such as [1H] tetrazole in a polar solvent, preferably an aprotic polar solvent. In this reaction first a phosphite is formed which may subsequently be oxidized to a phosphate for example in the presence of an aromatic peroxycarboxylic acid, such as m-chloroperbenzoic acid.

If R₄ is selected from (k) the process according to the invention may comprise sulfatation under suitable reaction conditions of the free hydroxyl group of the compound of the formula 4:

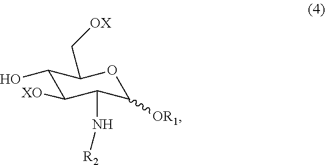

wherein R₁, R₂, and X are as defined before. This may be accomplished for example by reaction with a sulfur trioxide complex, for example trimethyl amine sulfur trioxide complex in a polar solvent such as DMF.

If R₄ is selected from (1), the process according to the invention may comprise reacting the free hydroxyl group of the compound of formula 4:

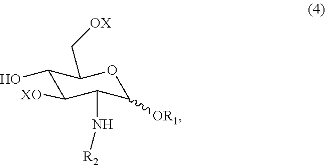

wherein R₁, R₂, and X are as defined before, with a compound suitable for donating a protecting group to said free hydroxyl group of the compound of formula 4. Such a protecting group donating compound may preferably be selected from benzyl-2,2,2-trichloroacetimidate or a substituted benzyl-2,2,2-trichloroacetimidate, such as 4-methoxybenzyl-2,2,2-trichloroacetimidate, 3,4-dimethoxybenzyl-2,2,2-trichloroacetimidate, 2,5-dimethoxybenzyl-2,2,2-trichloroacetimidate, 2,3,4-trimethoxybenzyl-2,2,2-trichloroacetimidate or 3,4,5-trimethoxybenzyl-2,2,2-trichloroacetimidate. Alternatively the protective group may be derived from a (C₃-C₆)alkenyl-2,2,2-trichloroacetimidate such as a C₃ or C₄-2,2,2-trichloroacetimidate, preferably a 2-propenyl-2,2,2-trichloroacetimidate or 1-propenyl-2,2,2-trichloroacetimidate. The reaction preferably is performed in a polar solvent and/or in the presence of an acid catalyst such as tin II trifluoromethanesulphonate or trifluoromethanesulphonic acid.

If R₄ is selected from (a) the process according to the invention may comprise reacting the free hydroxyl group of the compound of formula 4:

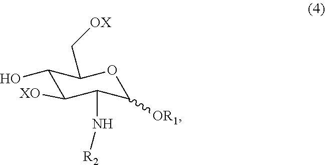

wherein R₁, R₂, and X are as defined before, with a carboxy group of a (activated) carboxylic acid of the formula R₄OH, wherein R₄ is selected from (a) as defined before. The reaction preferably is performed in the presence of a coupling agent such as isobutyl chloroformate or 1-isobutyloxy 2-isobutyloxycarbonyl-1,2-dihydroquinoleine or a carbodiimide.

If R₄ is selected from (b), (c), (d) or (e),) the process according to the invention may comprises reacting the free hydroxyl group of the compound of formula 4:

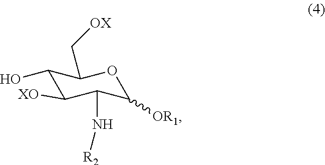

wherein R₁, R₂, and X are as defined before, with a 2,2,2, trichloroacetimidate activated alkyl alcohol derivative corresponding to said selection (b), (c), (d) or (e) of R₄. The reaction preferably is performed in a polar solvent and/or in the presence of an acid catalyst such as tin II trifluoromethanesulphonate or trifluoromethanesulphonic acid. The skilled person will understand that 2,2,2, trichloroacetimidate activated alcohol derivative corresponding to said selection (b), (c), (d) or (e) of R₄ may be an alkyl-2,2,2-trichloroacetimidate, such as e.g. propyl-2,2,2-trichloroacetimidate when R₄ is selected from (b) as an alkyl group. In analogy with this it is possible to select other 2,2,2, trichloroacetimidate activated alcohol derivatives corresponding to said selection (b), (c), (d) or (e) such as an alkenyl-2,2,2-trichloroacetimidate, alkynyl-2,2,2-trichloroacetimidate,

The various substituents of R₄ may similarly to the substituents of R₈ contain reactive groups, such as hydroxyl groups, amine groups, carboxy groups or carbon unsaturated bonds, such as double bonds. Such reactive groups on compound 5 may be further derivatized for example in a reaction selected from esterification, amidation, oxidation, hydrogenation or α, β hydroxylation with osmium tetroxide.

Compound 4 may be obtained by the reductive ring opening of the benzylidene group of a compound of the formula 3:

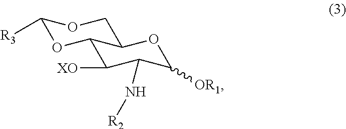

wherein R₁, R₂ and X are as defined previously, and R₃ is a group selected from an aromatic hydrocarbon, such as phenyl or 4-methoxyphenyl or 3,4-dimethoxyphenyl or 2,5-dimethoxyphenyl or 2,3,4-trimethoxyphenyl or 3,4,5-trimethoxyphenyl group. The reaction may be carried out with any method known in the art such as using a hydride, such as trimethylamine-borane complex, and a lewis acid, such as aluminum chloride, in a polar solvent, such as THF. This method is described in Carbohydrate Research, (2003), 697-703 and in Tetrahedron Lett. (2000), 41, 6843-6847.

Compound 10, which in the process of the invention is reacted together with compound 7 to form compound 11, may be obtained from compound 9:

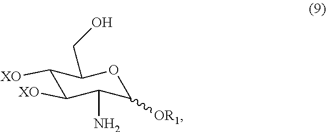

wherein R₁ and X are as defined previously. For formation of compound 10 the free amino group of compound 9 is acylated by reaction with an (activated) carboxylic acid of the formula R₅OH, wherein R₅ is as defined previously. The process may be carried out under conditions known to the skilled person with e.g. a mixed anhydride such as the mixed anhydride prepared from the (R)-3-benzyloxytetradecanoic acid described in Bull. Chem. Soc. Jpn, (1987), 2197-2204 and an alkyl chloroformate such as isobutyl chloroformate.

Compound 9 may be formed by the hydrolytic cleavage with known methods of the group R₂ of a compound of the formula 8:

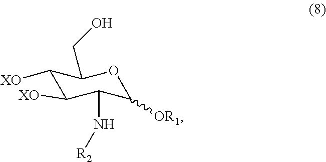

wherein R₁, R₂ and X are as defined previously. For example a trichloroethoxycarbonyl protective group (Troc) may be removed by using zinc in acetic acid.

The compound of formula 8, may be obtained by the reductive ring opening under suitable reaction conditions of the benzylidene group of a compound of the formula 3:

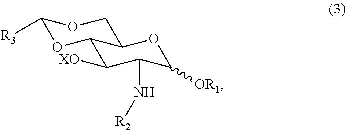

wherein R₁, R₂, R₃ and X are as defined previously. For this any method known in the art may be used such as using a hydride such as dimethylamine-borane complex as reagent and a Lewis acid such as boron-trifluoride in a polar solvent as dichloromethane.

The compound of the formula 3 may be obtained by reacting a compound of the

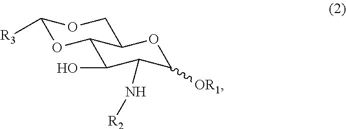

wherein R₁, R₂, R₃ and X are as defined previously with a compound suitable for donating a protecting group to the free hydroxyl group of the compound of formula 2. The protecting group donating compound preferably is selected from benzyl-2,2,2-trichloroacetimidate, 4-methoxybenzyl-2,2,2-trichloroacetimidate, 3,4-dimethoxybenzyl-2,2,2-trichloroacetimidate, 2,5-dimethoxybenzyl-2,2,2-trichloroacetimidate, 2,3,4-trimethoxybenzyl-2,2,2-trichloroacetimidate or 3,4,5-trimethoxybenzyl-2,2,2-trichloroacetimidate. The reaction preferably is performed in a polar solvent and/or in the presence of an acid catalyst such as tin II trifluoromethanesulphonate or trifluoromethanesulphonic acid. Suitable methods are disclosed in J. Chem Soc., Chem. Commun., (1981), 1240-1241). It is of interest to note that no reaction was observed using the methodology described in Tetrahedron Letters, (2001), 7613-7616 or in Tetrahedron Lett. (2000), 41, 6843-6847 to obtain the compound 3 and only the starting material 2 was recovered. As such these papers are considered to be non-enabling disclosures of compound 3. Compound 2 was prepared as described in Liebigs Ann. (1996), 1599-1607.

According to a further aspect the invention relates to a process for treating glucosamine disaccharides preferably β-(1→6)-linked glucosamine disaccharides. This process may be used to treat the compounds obtainable with the synthesis process according to the invention. The process comprises:
(i) mixing a solution of a compound of the formula 1:

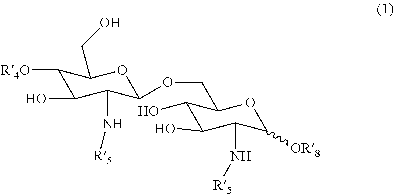

wherein R4′, R5′ and R8′ are as defined previously, with a solid reverse phase resin under conditions suitable for binding at least part of the compound of formula 1 to the solid phase;
(ii) removing the liquid phase and washing the solid phase with a washing liquid comprising an aqueous phase optionally buffered at pH 6-9, preferably 7-8, and most preferably 7.3-7.7, and an organic phase, which aqueous phase and organic phase are mixed in a ratio of between 15:1 to 5:1, preferably 9:1 (v/v);
(iii) removing the washing liquid and elution of at least part of the compound 1 bound to the solid phase with an elution liquid comprising an aqueous phase and an organic phase, which aqueous phase and organic phase are mixed in a ratio of between 1:15 to 1:5, preferably 1:9 (v/v);
(iv) collecting elution liquid comprising an amount of the compound of formula 1 and optionally removal of the organic phase from the elution liquid.

In a preferred embodiment the process further comprises adjusting the pH of the elution liquid comprising an amount of the compound of formula 1 to a pre-selected pH value, preferably to pH 6-9, more preferably pH 7-8, and most preferably pH 7.3-7.7. At this pH value the products are most stable.

Surprisingly it has been found that treating a compound of the formula 1 with this process results in compounds with an increased biological activity relative to the starting material.

The compounds of formula 1 may be bound to the reverse phase resin in a polar solvent such as a C₂-C₃ organic alcohol optionally mixed with water. Such as a mixture of water and 2-propanol, mixed in a ratio of 15:1 to 5:1, preferably 9:1 (v/v). The reverse phase resin may be VYDAC C18 resin or any other suitable reverse phase resin.

The organic phase of the washing liquid and/or the elution liquid may comprise an organic solvent such as a polar organic solvent for example a C₂-C₃ organic alcohol.

The compound of the formula 1 may be provided in a solvent which is suitable for the reaction wherein protective groups are removed by hydrogenolysis. An example of such a solvent is tetrahydrofurane (THF). As such the compounds according to the invention may be treated in the treatment process according to the invention directly after their synthesis with the process of the invention. However, it is preferred to first purify the compounds of the invention. Purification may be accomplished with methods known in the art such as by using reverse phase chromatography, preferably ion pair reverse phase chromatography such as with the use of tetrabutylammonium phosphate.

The compounds obtainable with the synthesis process according to the invention are β-(1→6)-linked glucosamine disaccharides according to the formula 1:

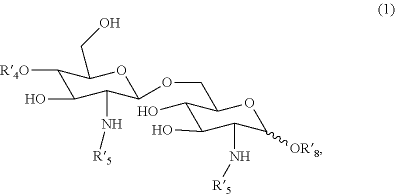

wherein R4′, R5′ and R8′ are as defined previously. One aspect of the invention relates to these compounds. Preferred compounds of the invention are presented in claim 47 and the figures attached. The skilled person will understand that these compounds may exist in ionized forms. The present invention also relates to (pharmaceutically acceptable) salts of such ionized forms, such as sodium, potassium or ammonium salts.

Many of the compounds according to the invention are novel with respect to their chemical structure. In addition to this the compounds according to the invention are distinguishable from compounds with a known chemical structure, but derived from natural sources due to the fact that they are free from any biological impurities such as traces of nucleic acids and/or peptides and/or carbohydrates. Although present in minute quantities the presence of traces of these biological impurities is considered unacceptable for pharmaceutical products. The presence of biological impurities may be determined with known methods for example selected from immunological methods or PCR methods. Such methods may in particular be aimed at detecting cellular components of gram-negative bacteria, such as E. coli.

In yet a different aspect the invention relates to certain novel intermediates of the process according to the invention. In particular according to this aspect the invention relates to compounds 3, 7, 8, 10a, 11, 11b, 12b, 12a, 13, 14. Preferred embodiments of this aspect of the invention relate to the compounds 3b, 7b, 8b, 10b, 11a, 11c, 12c, 12d, 13b, 14b. These compounds may be used as intermediates, including a starting material, in a process for the synthesis of an asymmetrically or symmetrically substituted β-(1→6)-linked glucosamine disaccharides.

The compounds according to formula 1 are of use in medicine for the treatment of warm-blooded animals such as mammals, including humans. In particular the compounds of the invention may be used in the treatment of immune disorders, such as immune disorders associated with overproduction of inflammatory cytokines or a decreased production of inflammatory cytokines. Inflammatory cytokines may be produced by activated T lymphocytes, monocytes, or antigen presenting cells and may belong to the group consisting of IL-1β, IL-4, IL-5 IL-6, IL-8, IL-9, IL-13, IFN-γ, TNF-α, or MCP-1. Conditions treatable with the compounds according to the invention include cancer, asthma, atopic dermatitis, allergic rhinitis, inflammatory bowel disease, diabetes, rheumatoid arthritis and others in which up- and/or down regulation of inflammatory cytokines is beneficial. The fact that the compounds of the invention preferentially act via human TLR2 may be of clinical interest to treat cancer (Garay et al., 2007). Cancers potentially treatable with the compounds of the invention include colorectal cancer, breast cancer and melanomas.

The compounds of the invention furthermore may decrease histamine secretion by mast cells. As such they are useful in the treatment, including amelioration, of conditions where excessive histamine secretion by mast cells is involved. Such conditions may include allergic reactions, including hay fever (pollinosis), allergic reactions caused by insect stings, such as bee stings and wasp stings or allergic reactions to food allergens.

Due to their stimulatory effect on the immune system the compounds of the invention furthermore are of use as vaccine components.

The compounds of the invention may be administered to a subject in need thereof in a formulation optionally in combination with a pharmaceutically-acceptable carrier and/or other excipients via the oral, parenteral, intravenous, intratumoral, subcutaneous, rectal, topical or mucosal routes. Administration via the peritoneal, subcutaneous, oral, intranasal, sublingual, intramuscular or aerosol routes is possible. Selection of suitable dosage ranges for the compounds of the invention will depend on the specific activity of the selected compound, the condition of the subject and the disorder treated. The skilled person will be able to select suitable dosage ranges based on his common general knowledge and his experience in the art. For conditions such as asthma, atopic dermatitis, allergic rhinitis, inflammatory bowel disease, diabetes or rheumatoid arthritis suitable dosage ranges for humans may be from 0.01 to 50 mg/m².

Further aspects of the invention relate to processes wherein the novel and inventive (intermediate) compounds of the invention are used and/or synthesized. Due to the use and/or production of novel and inventive compounds these processes are novel and inventive. The processes may be of use in the synthesis of an asymmetrically or symmetrically substituted 1,6-β disaccharide including the compound of the invention.

The invention will now be further illustrated with reference to the following examples and the accompanying figures, wherein:

FIG. 1 shows the structure of E. coli Lipid A and OM-174-DP®;

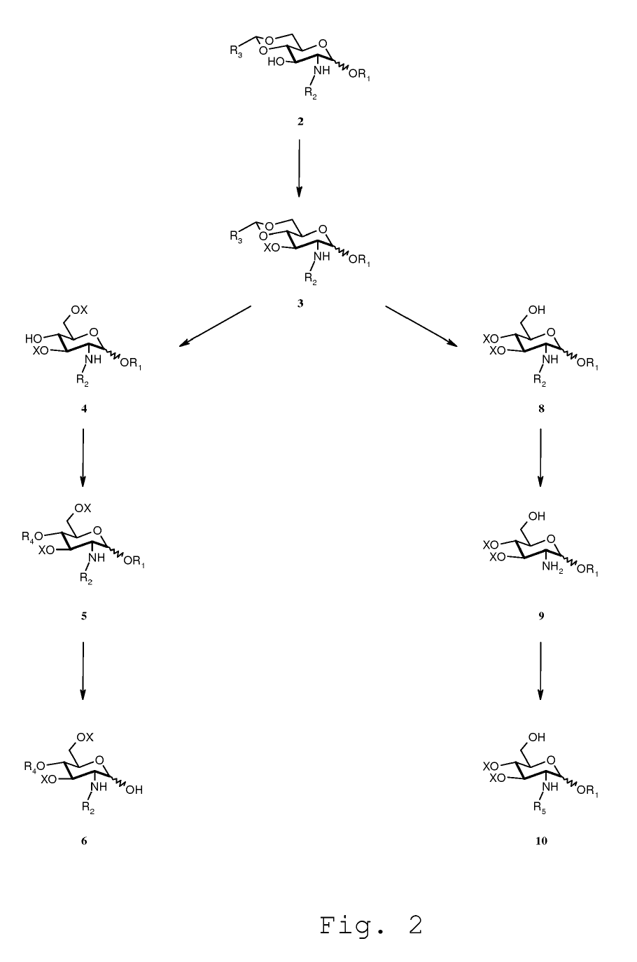

FIG. 2, gives an overview of an embodiment of the synthesis process according to the invention;

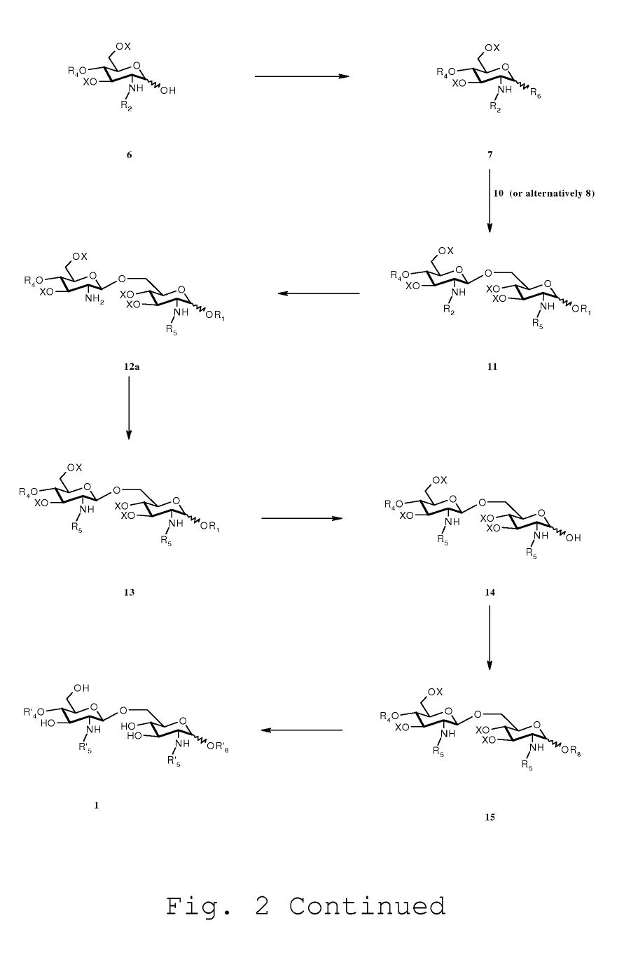

FIG. 3, gives an overview of a preferred embodiment of the synthesis process according to the invention;

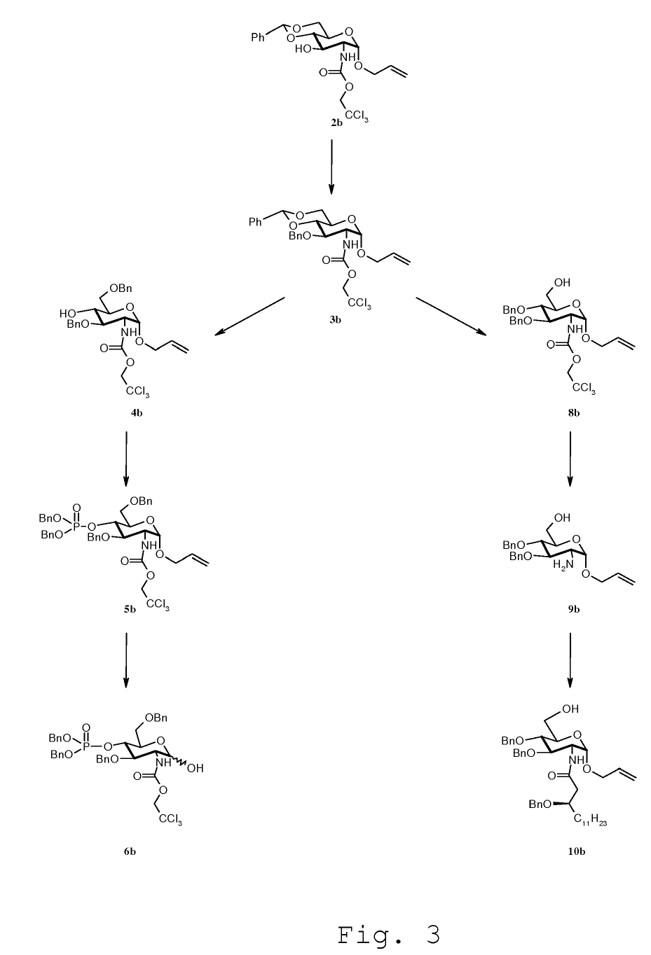

FIGS. 4-24, give an overview of various alternative synthesis routes for forming compounds of the formula 1 and/or direct predecessors thereof;

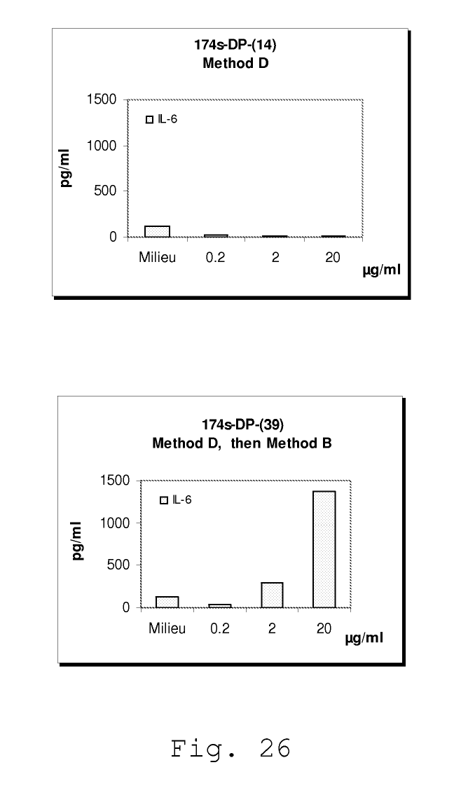

FIG. 25 represents a graph showing NO production by murine macrophages in response to compounds of the invention;

FIG. 26 represents experimental results illustrating the enhancement of the biological activity of β-(1→6)-linked glucosamine disaccharides when treated with the method according to the invention.

In these figures the groups R₀, R₁, R₂, R₄, R₅, R₆, R₈, R₄′, R₅′, R₈′, X, Y and W are as defined in the claims and the description for the various compounds. Bn designates a benzyl group, Allyl designates an allyl group and Ipr designates an isopropyl group.

The molecular structures represented in FIG. 1 correspond to E. coli Lipid A and OM-174-DP® as indicated. In FIG. 1 the designation of O-3 and O-3′ are furthermore indicated.

FIG. 2, gives an overview of an embodiment of the synthesis process according to the invention. From the description above it will be clear that compound 7 may be reacted with compound 10 to obtain compound 11h, wherein R₀ is R₅ or alternatively with compound 8 to obtain compound 11h, wherein R₀ is selected from R₂. In the embodiment shown in FIG. 2, compound 7 is reacted with compound 10. This opens the possibility to introduce different R₅ substituents on the molecule which thus may be asymmetrically substituted. Symmetrically substituted compounds may be obtained by reacting compound 7 with compound 8 and subsequently reacting the obtained compound 11h wherein R₀ is selected from R₂ to a compound 12b. With compound 12b the reaction sequence may be proceeded in a similar fashion in order to obtain compounds which are symmetrically substituted at the N-2 and N-2′ position.

FIG. 3, gives an overview of a preferred embodiment of the synthesis process according to the invention. In this embodiment of the process according to the invention the asymmetrically substituted OM-174-DP® is the endproduct.

FIG. 4 shows a first possible reaction for phosphorylation of the free hydroxyl group of the hemiacetal of formula 14. In this reaction compound 14 is reacted with tetrabenzyl pyrophosphate in the presence of lithium bis(trimethylsilyl)amide (LiHMDS). The reaction may take place in a polar solvent such as THF.

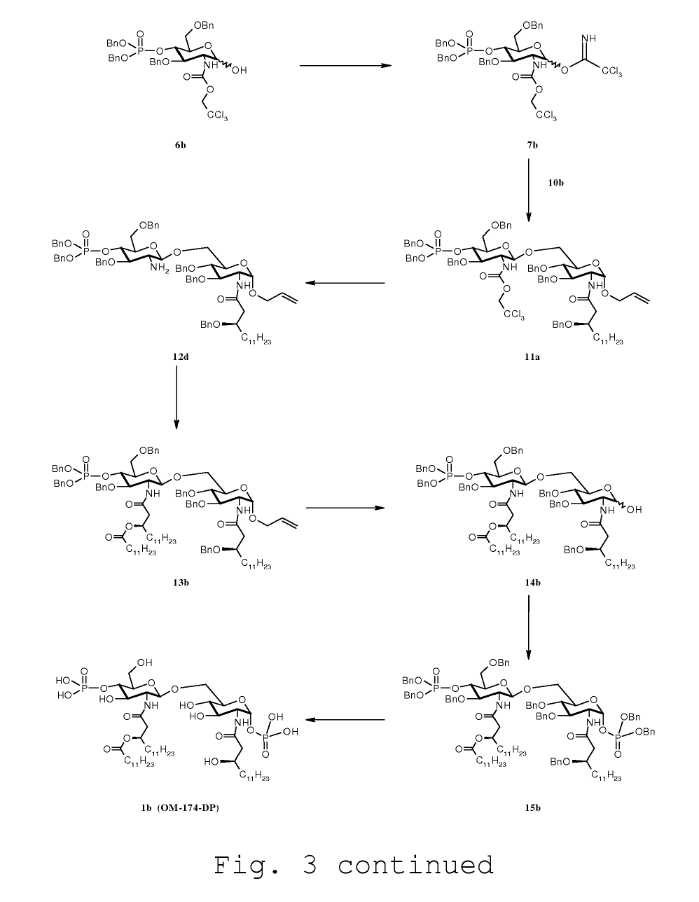

FIG. 5 shows an alternative reaction for phosphorylation of the free hydroxyl group of the hemiacetal of formula 14. In this reaction compound 14 is reacted with diallyl N,N-diisopropyl phosphoramidite in the presence of a coupling agent, such as [1H] tetrazole. The reaction may take place in a polar solvent, preferably an aprotic polar solvent. In the reaction first a phosphite is formed. This phosphite is subsequently oxidized to a protected phosphate in the presence of an aromatic peroxycarboxylic acid, such as m-chloroperbenzoic acid.

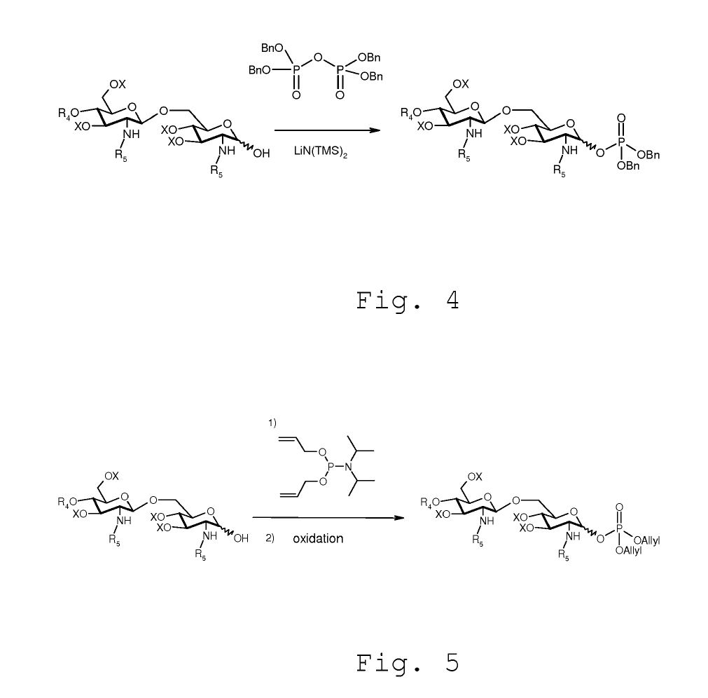

FIG. 6 shows the exemplary formation of a phosphodiester by reaction of a phosphonate with a protected organic amino alcohol of the formula HO—(C₁-C₂₄)—NHW. After formation of the phosphodiester bond the protecting group W may be removed together or separately from the protecting groups X. When the group W is removed, while the groups X remain on the molecule, the free amino group may be further derivatised, e.g. by forming an amide with an organic acid.

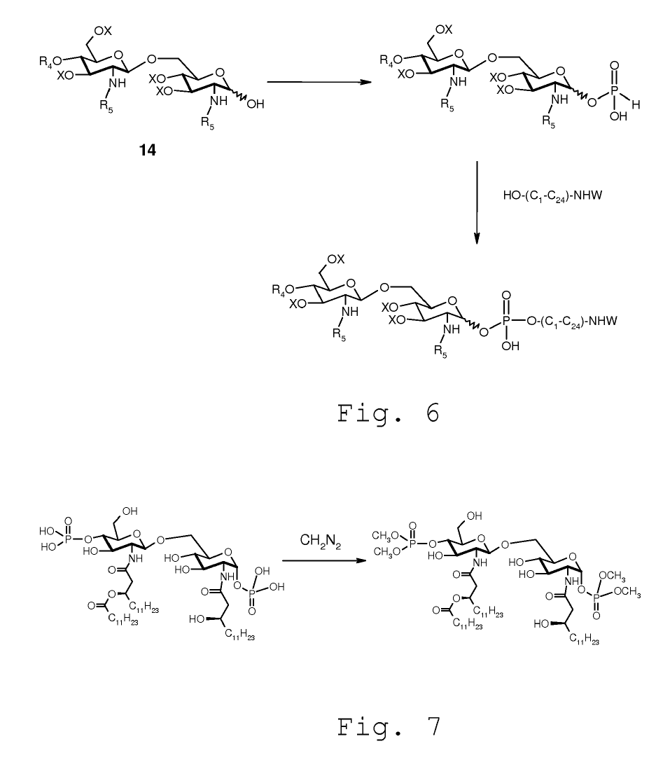

FIG. 7 shows a further alternative reaction for derivatisation of a phosphate group. In this reaction the phosphate group is methylated with CH₂N₂. The reaction shown in FIG. 7 is performed on a molecule wherein neither of the phosphate groups is protected. It will be understood that when one of the phosphate groups is protected, such as the 1-O phosphate group, or the 4′-O phosphate group such a protected phosphate group will not be methylated in the reaction. This opens the possibility for selective derivatisation of either or both phosphate groups.

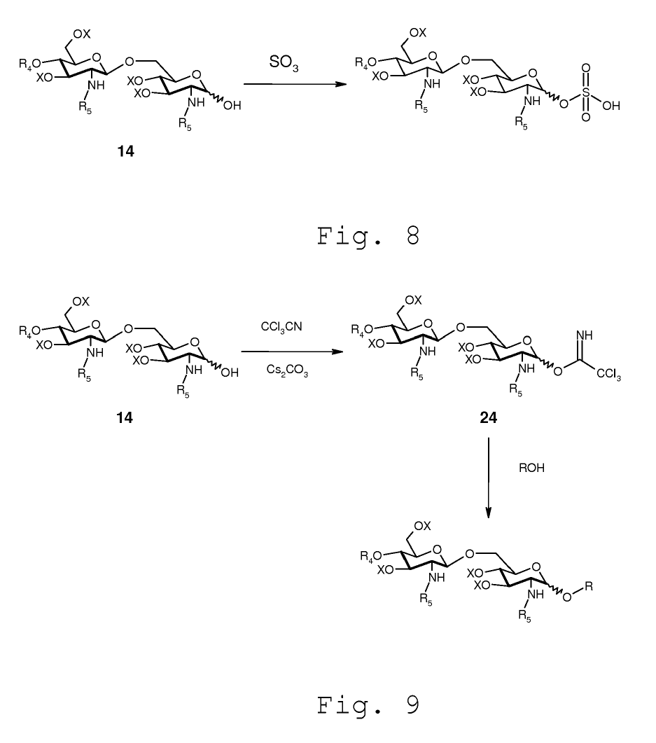

FIG. 8 shows the reaction for sulfatation of compound 14. In the reaction compound 14 is reacted with sulfur trioxide complex.

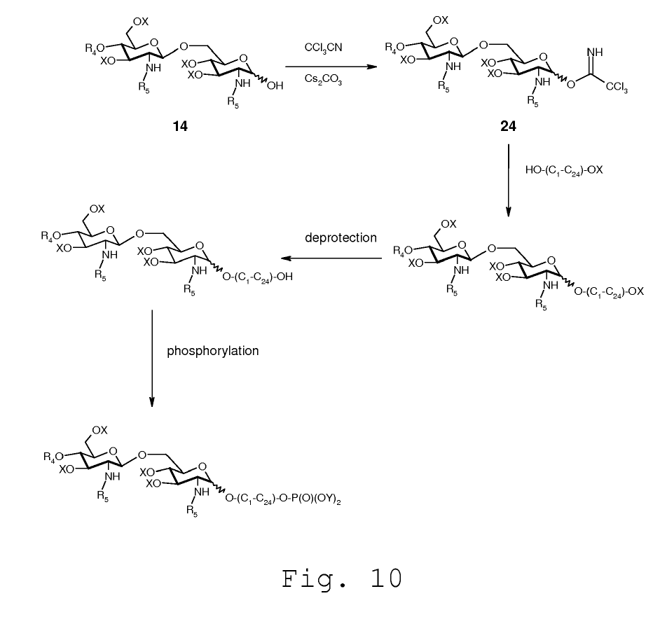

In order to obtain compounds having a hydrocarbon chain attached directly to the 1-O position there are a number of possibilities. Some of these are shown in FIG. 9. First it is possible to hydrogenate the (C₃-C₆) alkenyl attached to the 1-O position in the compound of the formula 13 to the corresponding alkyl. The 1-allyl group is hydrogenated to a propyl group. Secondly it is possible to attach hydrocarbon chains by first activating the hydroxyl function at the 1-O position of compound 14 and subsequent reaction of the activated group with an organic alcohol. Activation of the free hydroxyl group of compound 14 may be achieved by reaction of compound 14 with trichloacetonitrile in the presence of a mineral base, such as cesium carbonate or potassium carbonate. This reaction may take place in a polar solvent, preferably an aprotic polar solvent such as dichloromethane. When compound 14 is reacted with trichloacetonitrile under such conditions a compound of the formula 24 will be formed. Reaction of compound 24 with an organic alcohol represented with the general formula ROH in FIG. 9, will result is a compound having the hydrocarbon chain R attached to the O-1 position.

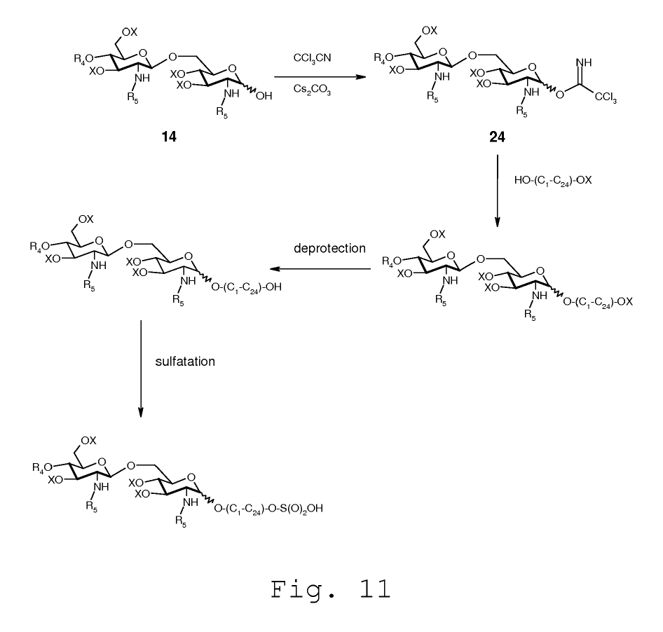

FIG. 10 shows a further example of a reaction of compound 24 with an organic alcohol. In FIG. 10, compound 24 is reacted with an organic diol having 1 to 24 carbon atoms of which one of the hydroxyl groups is protected with a group X, preferably PMB. The monoprotected organic diol is represented with the generic formula HO—(C₁-C₂₄)—OX. In FIG. 10 it is furthermore shown that after coupling of the monoprotected organic diol to the O-1 position, the protecting group X of the monoprotected organic diol may be removed selectively if it is selected differently from the group X on the carbohydrate. After selective removal of the protecting group X of the monoprotected organic diol, the free hydroxyl group may be further derivatised e.g. by phosphorylating it with methods discussed above. It will be understood that the phosphate group may be further derivatised as discussed above.

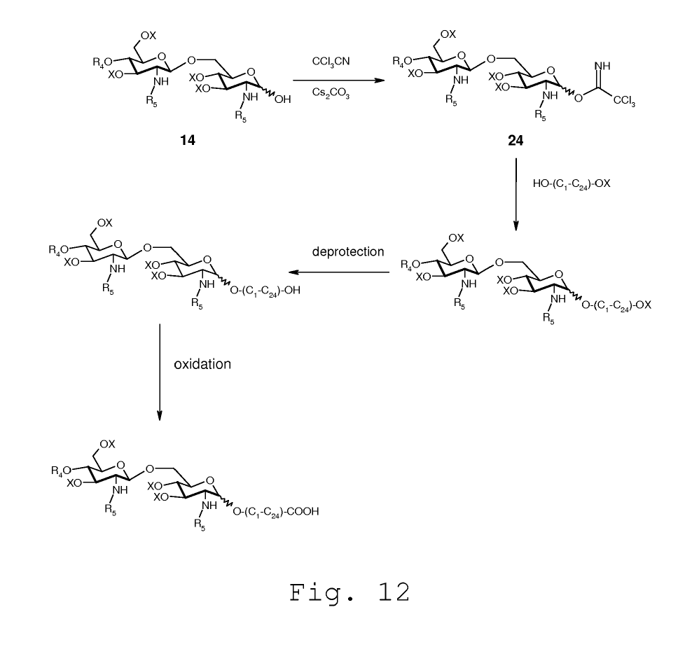

FIG. 11 shows a reaction scheme similar to that of FIG. 10. However, in FIG. 11 after removal of the protective group X of the monoprotected organic diol, the hydroxyl group is subjected to sulfatation.

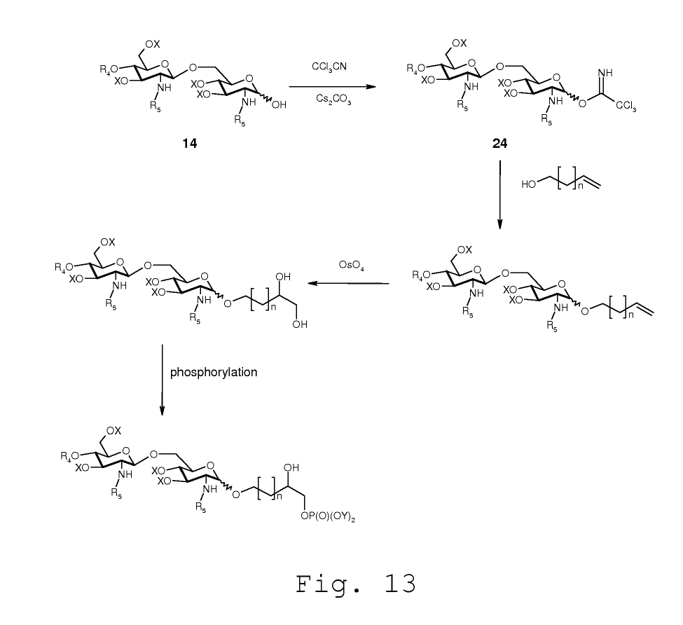

Alternatively, as shown in FIG. 12, after removal of the protective group X of the monoprotected organic diol, the hydroxyl group may be oxidized to a carboxy group. It will be understood that the carboxy group may be further derivatised e.g. by formation of an amide or an ester.

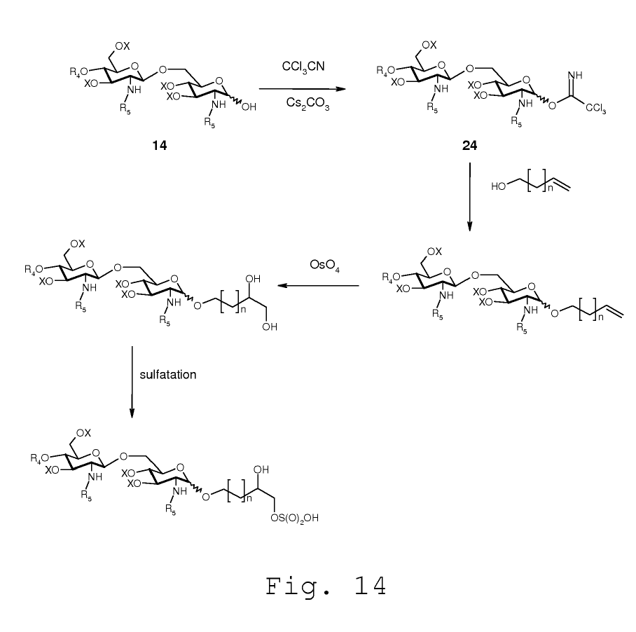

FIG. 13 shows a reaction sequence which makes it possible to introduce a hydrocarbon chain having an α, β dihydroxy substitution. In this reaction scheme an organic alcohol having an unsaturated carbon-carbon double bond is reacted with compound 24. The length of the hydrocarbon chain connecting the hydroxyl group and the unsaturated bond of the organic alcohol shown is variable and comprises n carbon atoms, wherein n may vary between 1 and 24. Although the unsaturated bond of the organic alcohol shown is present at the terminus of the organic alcohol it will be understood that it may also be present at a location within the hydrocarbon chain. After connection of the organic alcohol to the 1-O position of compound 24 the unsaturated bond may be reacted with osmium tetroxide for α, β dihydroxy addition to the double bond. The hydroxyl groups introduced in this way may be further derivatised. For example by formation of phosphate as shown in FIG. 13 or alternatively by formation of sulfate, esters with organic acids or ethers. In FIG. 13 only a single hydroxyl group is phosphorylated. This may be achieved by reaction with a minor amount of the phosphorylation reagent. It will be understood that in such a reaction the bisphosphate will also be formed.

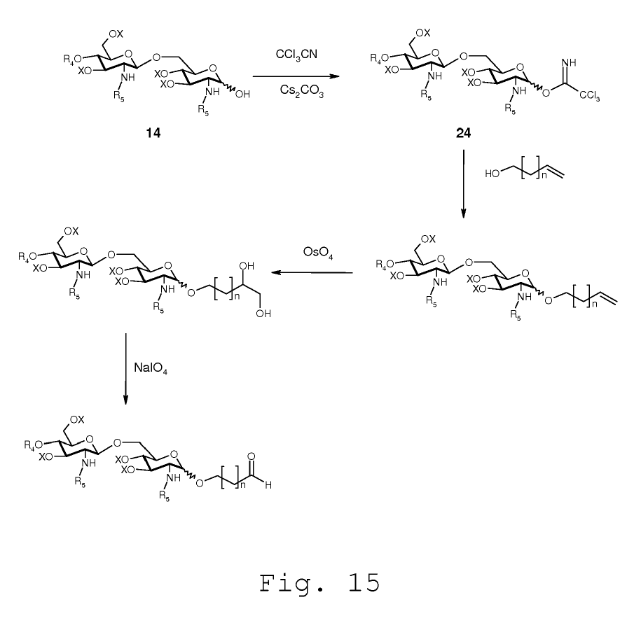

FIG. 14 shows a reaction sequence similar to the reaction sequence shown in FIG. 13. However, after α, β dihydroxy addition to the double bond the hydroxyl functions are sulfated.

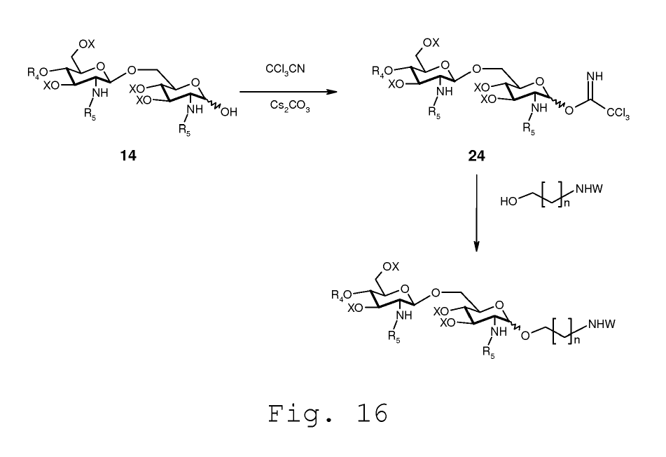

FIG. 15 shows a reaction sequence similar to the reaction sequence shown in FIG. 13. However, after α, β dihydroxy addition to the double bond the hydroxyl functions are reacted with a oxidising agent such as NaIO₄ to obtain a carbonyl function.

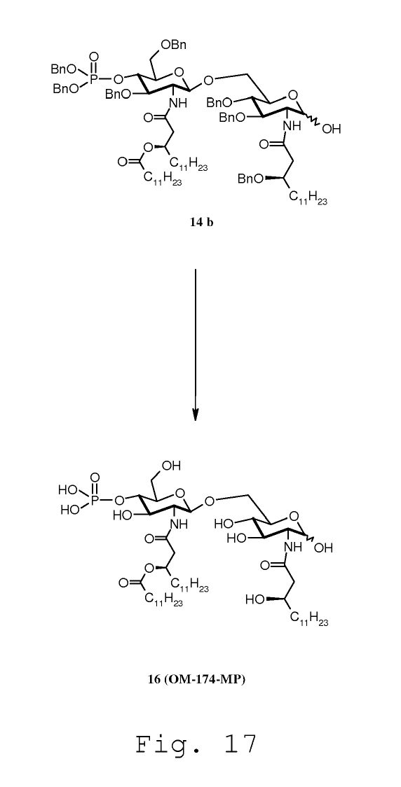

In the reaction scheme shown in FIG. 16, compound 24 is reacted with a protected organic amino alcohol of the formula HO—(C₁-C₂₄)—NHW. After connection of the protected organic amino alcohol the protected amine function may be further treated as discussed in connection to FIG. 6.

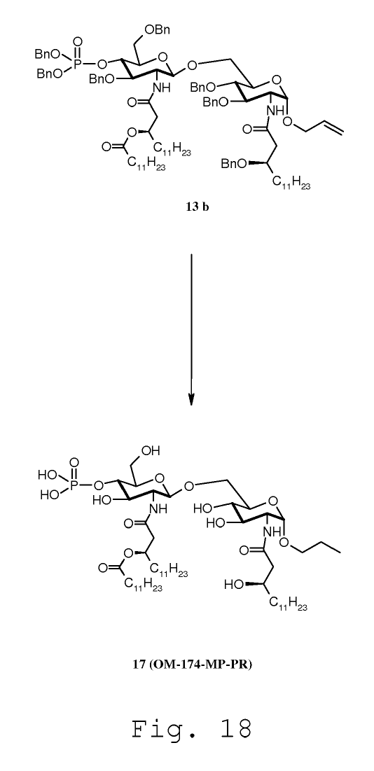

FIG. 17 shows part of the reaction sequence for obtaining compound OM-174-MP (compound 16) from compound 14b. Details of the reaction sequence are provided in the synthesis examples.

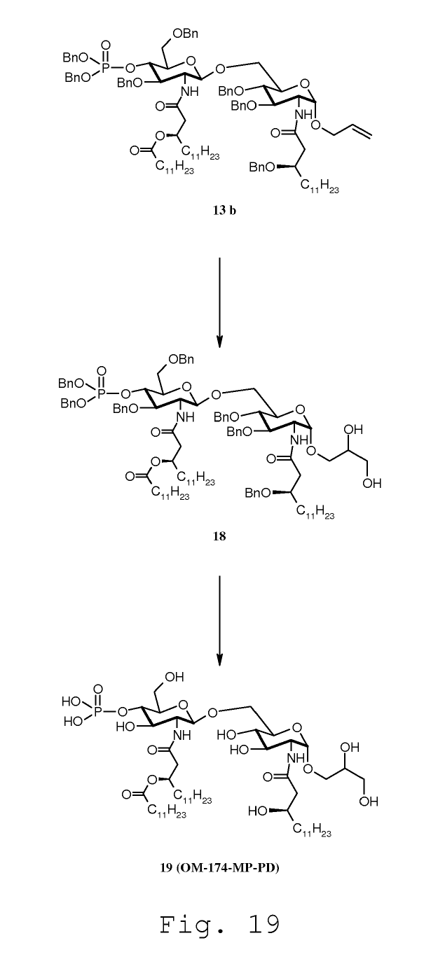

FIG. 18 shows part of the reaction sequence for obtaining compound OM-174-MP-PR (compound 17) from compound 14b. Details of the reaction sequence are provided in the synthesis examples.

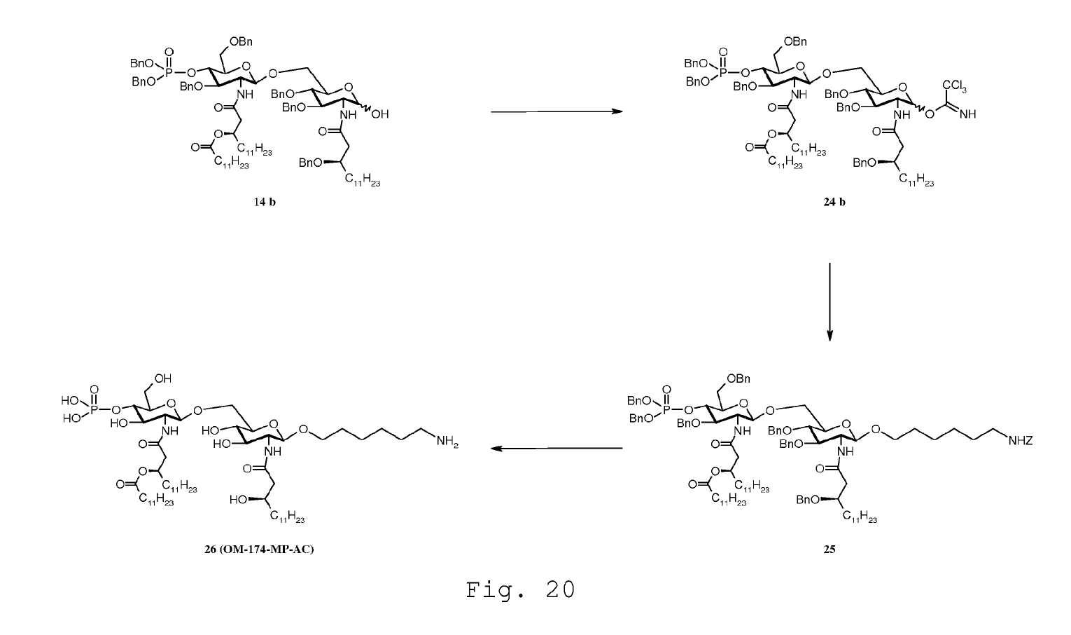

FIG. 19 shows part of the reaction sequence for obtaining compound OM-174-MP-PD (compound 19) from compound 13b via compound 18. Details of the reaction sequence are provided in the synthesis examples.

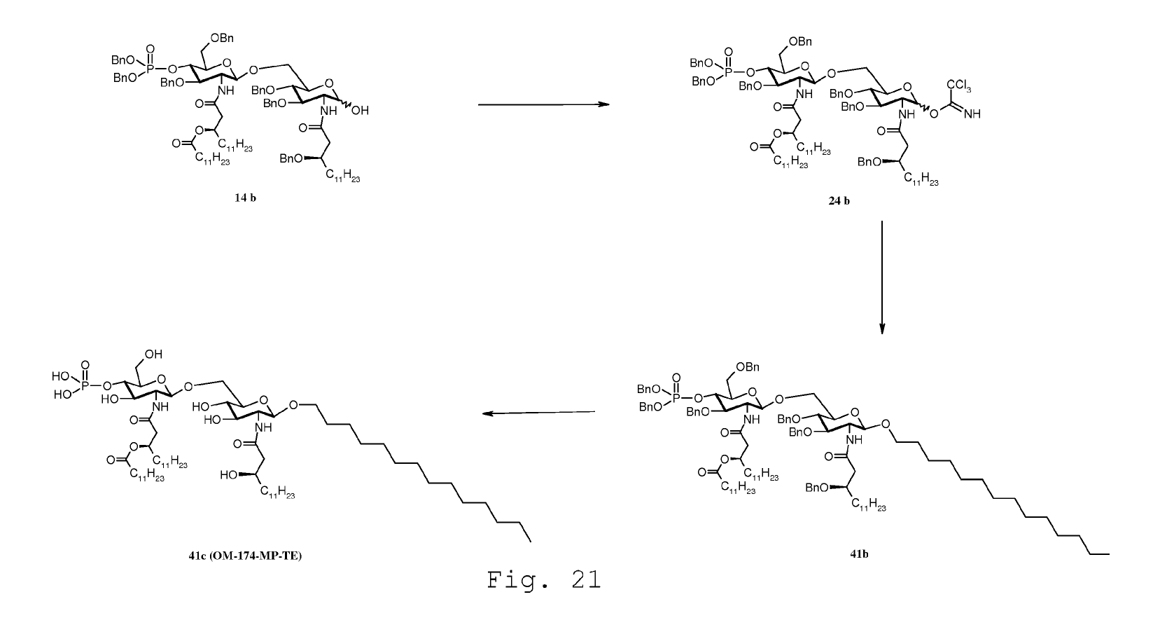

FIG. 20 shows a reaction sequence for obtaining compound OM-174-MP-AC (compound 26) starting from compound 14b. Details of the reaction sequence are provided in the synthesis examples.

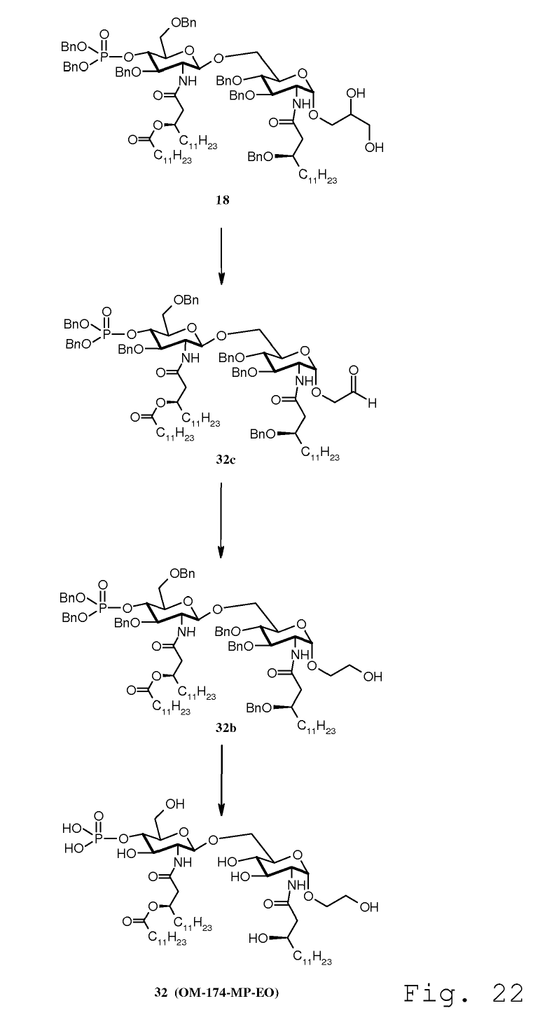

FIG. 21 shows a reaction sequence for obtaining compound OM-174-MP-TE (compound 41c) from compound 14b. Details of the reaction sequence are provided in the synthesis examples.

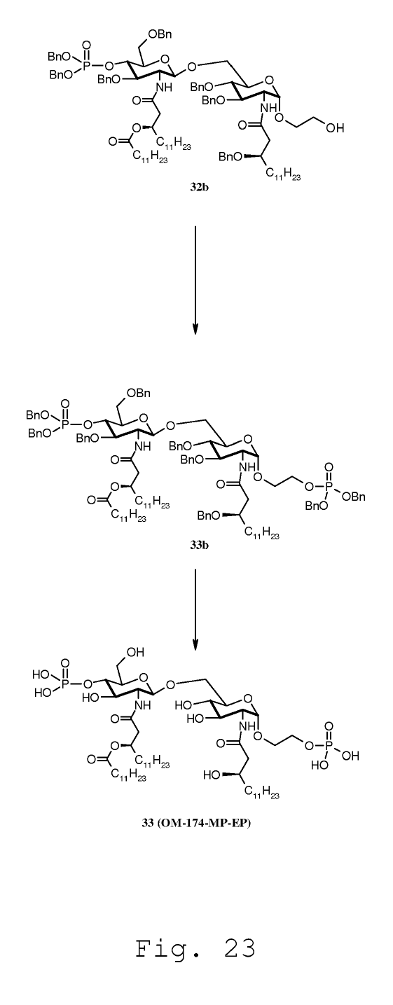

FIG. 22 shows a reaction sequence for obtaining compound OM-174-MP-EO (compound 32) from compound 18. Details of the reaction sequence are provided in the synthesis examples.

FIG. 23 shows a reaction sequence for obtaining compound OM-174-MP-EP (compound 33) from compound 32b. Details of the reaction sequence are provided in the synthesis examples.

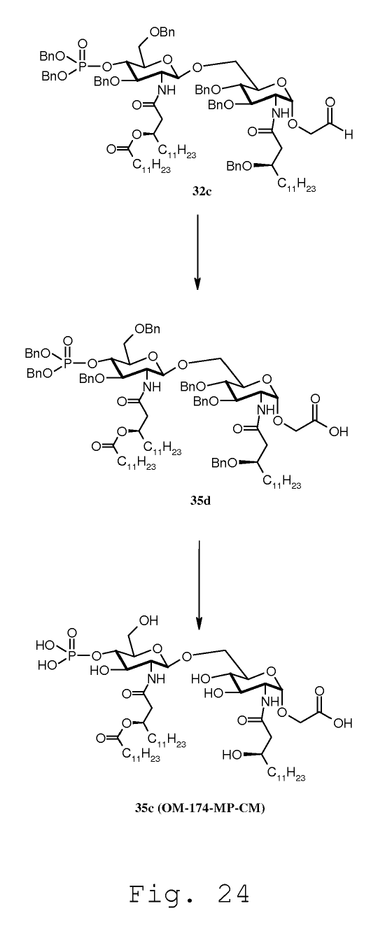

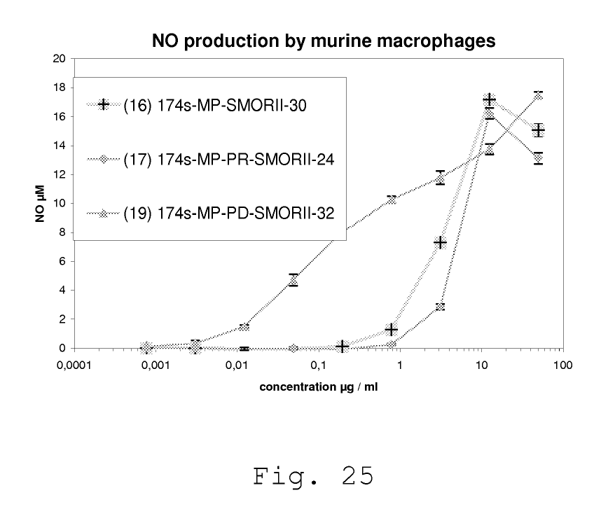

FIG. 24 shows a reaction sequence for obtaining compound OM-174-MP-CM (compound 35c) from compound 32c. Details of the reaction sequence are provided in the synthesis examples.

The reactions discussed above may also be used to connect different substituents to the O-4′ position of the β-(1→6)-linked glucosamine disaccharides of the invention. This may be achieved by using the reactions discussed above for introduction of substituents to the O-1 position. These reactions may similarly be performed on the free hydroxyl group of the compound of formula 4.

Form the above it will be clear that a great diversity of substitutions may be connected to the O-1 and O-4′ positions of the β-(1→6)-linked glucosamine disaccharides of the invention.

In the following experimental examples of the synthesis of the compounds of the invention and examples relating to the biological activity of the compounds of the invention will be provided.

### SYNTHESIS EXAMPLES

In the following section the synthesis of compounds of the invention will be discussed. The various compounds synthesized are shown in FIG. 3 with their corresponding compound designation number.

### Allyl-3-O-benzyl-4,6-O-benzylidene-2-deoxy-2-(2,2,2-trichloroethoxycarbonylamino)-α-D-glucopyranoside (3b)

To a stirred suspension of Allyl-4,6-O-benzylidene-2-deoxy-2-(2,2,2-trichloroethoxycarbonylamino)-α-D-glucopyranoside 2b [Liebigs Ann. (1996), 1599-1607] (5 g, 10.35 mmol) and commercially available benzyl 2,2,2-trichloroacetimidate (2.9 mL, 15.5 mmol) in ether (200 mL) was added tin trifluoromethanesulfonate (863 mg, 2.1 mmol). The mixture was stirred for 17 h at room temperature, neutralized with saturated NaHCO₃ and concentrated. The residue was taken up in EtOAc, washed with H₂O and the organic phase was separated and dried over MgSO₄. The solvent was evaporated and the residue recrystallized from EtOH to give 3b (4.43 g, 75%) as a white crystalline solid. Mp 168.7° C.; [α]_{D}+65 (c 0.24, CHCl₃); νₘₐₓ cm⁻¹ 3301, 2915, 1709, 1546, 1075, 1013, 693; ¹H NMR (500 MHz, CDCl₃): δ 7.54-7.26 (m, 10H, Ph), 5.90 (m, 1H, CH═CH₂), 5.62 (s, 1H, PhCH), 5.32-5.22 (m, 2H, CH═CH₂), 5.10 (d, 1H, J_{2,NH} 10.0 Hz, NH), 4.95 (AB, 1H, J 11.9 Hz, CH₂Ph), 4.92 (d, 1H, J_{1,2} 3.7 Hz, H-1), 4.81 (d, 1H, J 12.0 Hz, CH₂CCl₃), 4.71 (AB, d, 2H, CH₂Ph, CH₂CCl₃), 4.30 (dd, 1H, J_{6,6′} 10.1 Hz, J_{6,5} 4.6 Hz, H-6), 4.19 (m, 1H, OCH₂CH), 4.07-3.97 (m, 2H, H-2, OCH₂CH), 3.88 (m, 1H, H-5), 3.83-3.75 (m, 3H, H-6′, H-4, H-3); ¹³C NMR (125.8 MHz, CDCl₃): δ 154.3 (C═O), 138.2 (Cq), 137.2 (Cq), 133.2 (CH═CH₂), 129.0, 128.7, 128.2, 126.0 (CH arom), 118.3 (CH═CH₂), 101.2 (PhCH), 97.3 (C-1), 95.4 (CH₂CCl₃), 82.7 (C-4), 76.2 (C-3), 74.8-74.4 (CH₂CCl₃, CH₂Ph), 68.9 (C-6), 68.6 (OCH₂CH), 62.9 (C-5), 54.9 (C-2); MS-ES 596-594 [M+Na]⁺; Anal. Calcd. for C₂₆H₂₈Cl₃NO₇: C, 54.51; H, 4.93; N, 2.44%. Found: C, 54.51; H, 4.94; N, 2.34%.

### Allyl-3,6-di-O-benzyl-2-deoxy-2-(2,2,2-trichloroethoxycarbonylamino)-α-D-glucopyranoside (4b)

To a stirred solution of 3b (1.3 g, 2.27 mmol) and borane trimethylamine complex (660 mg, 9.08 mmol) in dry THF (45 mL) at room temperature was added aluminium chloride (1.81 g, 13.6 mmol). After the dissolution of the reagents, water (82 μl, 4.54 mmol) was added dropwise and stirring was continued at room temperature for 30 min. The mixture was stopped by addition of water (20 mL) followed by 1M HCl solution (20 mL) and diluted with EtOAc. The organic phase was separated, washed with a saturated solution of NaCl, dried over MgSO₄ and the solvent removed in vacuo. Flash chromatography of the residue on silica gel (n-heptane/EtOAc, 3:1) provided compound 4b (1.13 g; 87%) as a white solid. Mp 65° C.; [α]_{D}+64 (c 0.80, CHCl₃); νₘₐₓ cm⁻¹ 3329, 2915, 1706, 1536, 1044, 730, 694; ¹H NMR (500 MHz, CDCl₃): δ 7.40-7.26 (m, 10H, Ph), 5.90 (m, 1H, CH═CH₂), 5.32-5.21 (m, 2H, CH═CH₂), 5.14 (d, 1H, J_{2,NH} 10.0 Hz, NH), 4.92 (d, 1H, J_{1,2} 3.7 Hz, H-1), 4.82 (d, 1H, J 12.0 Hz, CH₂CCl₃), 4.80 and 4.77 (AB, 2H, J 11.6 Hz, CH₂Ph), 4.67 (d, 1H, J 12.0 Hz, CH₂CCl₃), 4.64 and 4.57 (AB, 2H, J 12.0 Hz, CH₂Ph), 4.19 (m, 1H, OCH₂CH), 4.03-3.97 (m, 2H, H-2, OCH₂CH), 3.82-3.68 (m, 4H, H-6, H-6′, H-5, H-4), 3.63 (t, 1H, J_{2,3}=J_{3,4} 10.2 Hz, H-3), 2.60 (s, 1H, OH); ¹³C NMR (125.8 MHz, CDCl₃): δ 154.2 (C═O), 138.2 (Cq), 137.7 (Cq), 133.4 (CH═CH₂), 128.8, 128.5, 128.4, 127.8, 127.7, 127.6 (CH arom), 118.0 (CH═CH₂), 96.8 (C-1), 95.4 (CH₂CCl₃), 80.2 (C-3), 74.6, 74.5, 73.6 (CH₂CCl₃, 2×CH₂Ph), 72.0, 70.2 (C-4, C-5), 69.7 (C-6), 68.3 (OCH₂CH), 54.5 (C-2); MS-ES 598-596 [M+Na]⁺; Anal. Calcd. for C₂₆H₃₀Cl₃NO₇: C, 54.32; H, 5.26; N, 2.44%. Found: C, 54.55; H, 5.39; N, 2.39%.

### Allyl-3,6-di-O-benzyl-4-O-(dibenzyloxyphosphoryl)-2-deoxy-2-(2,2,2-trichloroethoxycarbonylamino)-α-D-glucopyranoside (5b)

To a stirred solution of 4b (1.1 g, 1.91 mmol) and a commercially available solution of 1H-tetrazole in CH₃CN (˜0.45 M) (8.5 mL, 3.8 mmol) in CH₂Cl₂ (33 mL) at room temperature was added dibenzyl dimethylphosphoramidite (762 μl; 2.87 mmol). Stirring was continued at room temperature for 30 min and the solution was then cooled down to −20° C. A solution of mCPBA (57-86%, 1.22 g; 7.00 mmol) in CH₂Cl₂ (20 mL) was added and the solution was stirred for 30 min at −20° C. 10% Aqueous sodium thiosulfate (50 mL) was added and the mixture was stirred for 10 min, then diluted with EtOAc and the organic phase was separated. The organic layer was washed successively with 10% aqueous Na₂S₂O₃ solution (3×), saturated aqueous NaHCO₃ solution (2×), N HCl solution (1×) and brine. The organic phase was dried over MgSO₄ and the solvent removed in vacuo. Flash chromatography of the residue on silica gel (n-heptane/EtOAc, 4:1) provided compound 5b (1.25 g; 78%) as a colorless oil. [α]_{D}+56 (c 1.32, CHCl₃); νₘₐₓ cm⁻¹ 3301, 2920, 1728, 1542, 1453, 1264, 995, 731, 694; ¹H NMR (500 MHz, CDCl₃): δ 7.40-7.10 (m, 20H, Ph), 5.90 (m, 1H, CH═CH₂), 5.33-5.23 (m, 2H, CH═CH₂), 5.13 (d, 1H, J_{2,NH} 10.0 Hz, NH), 4.93 (d, 1H, J_{1,2} 3.5 Hz, H-1), 4.96-4.82 (m, 4H, 2×CH₂Ph), 4.86 (m, 1H, CH₂CCl₃), 4.76 and 4.65 (AB, 2H, J 12.0 Hz, CH₂Ph), 4.73 (m, 1H, CH₂CCl₃), 4.60 (t, 1H, J_{3,4}=J_{4,5} 9.5 Hz, H-4), 4.57 and 4.47 (AB, 2H, J 12.0 Hz, CH₂Ph), 4.21 (m, 1H, OCH₂CH), 4.10 (ddd, 1H, H-2), 4.02 (m, 1H, OCH₂CH), 3.92 (m, 1H, H-5), 3.84 (dd, 1H, J_{2,3} 9.3 Hz, H-3), 3.80 (dd, 1H, J_{6,5} 2.0 Hz, J_{6,6′} 11.0 Hz, H-6), 3.76 (dd, 1H, J_{6′,5} 4.7 Hz, H-6′); ¹³C NMR (125.8 MHz, CDCl₃): δ 154.0 (C═O), 138.1 (Cq), 137.8 (Cq), 135.8 (Cq), 135.7 (Cq), 133.2 (CH═CH₂), 128.6, 128.5, 128.4, 128.3, 128.2, 128.1, 128.0, 127.9, 127.8, 127.7, 127.6, 127.5, 127.4 (CH arom), 118.1 (CH═CH₂), 96.4 (C-1), 95.3 (CH₂CCl₃), 78.4 (C-3), 75.6 (C-4), 74.6, 73.7, 73.3 (CH₂CCl₃, 2×CH₂Ph), 70.2 (C-5), 69.5, 69.4 (2×CH₂Ph), 68.4 (C-6, OCH₂CH), 54.3 (C-2); MS-ES 858-856 [M+Na]⁺; Anal. Calcd. for C₄₀H₄₃Cl₃NO₁₀P: C, 57.53; H, 5.19; N, 1.68%. Found: C, 57.41; H, 5.28; N, 1.74%.

### 3,6-Di-O-benzyl-4-O-(dibenzyloxyphosphoryl)-2-deoxy-2-(2,2,2-trichloroethoxycarbonylamino)-D-glucopyranose (6b)

To a stirred solution of 5b (659 mg; 0.79 mmol) in dry THF (10 mL) at room temperature was added [bis(methyldiphenylphosphine)]-(1,5-cyclooctadiene)Iridium(I) hexafluorophosphate (67 mg). After activation of the iridium catalyst with hydrogen for 1 min (the slightly red solution becomes colorless), the mixture was stirred under nitrogen for 1 h. Iodine (360 mg, 1.42 mmol) and water (850 μL) were added and the reaction mixture was stirred for additional 30 min. To the mixture was added 10% aqueous Na₂S₂O₃ solution and the solution was extracted with EtOAc. The organic layer was washed successively with 10% aqueous Na₂S₂O₃ solution (2×) and brine. The organic phase was dried over MgSO₄, the solvent removed in vacuo and the residue crystallized from a mixture n-heptane/EtOAc to give 6b (419 mg, 67%) as a pale yellow solid. νₘₐₓ cm⁻¹ 3361, 2920, 1716, 1522, 1452, 1216, 1006, 729, 693; ¹H NMR (500 MHz, CDCl₃) for α-anomer: δ 7.40-7.12 (m, 20H, Ph), 5.20 (d, 1H, J_{1,2} 3.4 Hz, H-1), 5.17 (d, 1H, J_{2,NH} 9.8 Hz, NH), 4.96-4.40 (m, 10H, 4×CH₂Ph, CH₂CCl₃), 4.45 (t, 1H, J_{3,4}=J_{4,5}9.5 Hz, H-4), 4.15 (m, 1H, J_{6,5}6.4 Hz, J_{4,5} 9.5 Hz, H-5), 4.00 (dt, 1H, J_{2,NH}=J_{2,3} 9.8 Hz, H-2), 3.78 (dd, 1H, H-3), 3.78 (dd, 1H, J_{6′,5} 1.7 Hz, J_{6,6′} 11.0 Hz, H-6′), 3.76 (dd, 1H, J_{6,5} 6.4 Hz, H-6); ¹³C NMR (125.8 MHz, CDCl₃) for α-anomer: δ 154.1 (C═O), 137.8 (Cq), 137.7 (Cq), 135.7 (Cq), 135.6 (Cq), 128.6, 128.5, 128.4, 128.3, 128.2, 128.1, 128.0, 127.9, 127.8, 127.7, 127.6, 127.5, 127.4 (CH arom), 95.3 (CH₂CCl₃), 91.6 (C-1), 77.9 (C-3), 76.1 (C-4), 74.6, 73.7, 73.3 (CH₂CCl₃, 2×CH₂Ph), 70.6 (C-5), 69.5, 69.4 (2×CH₂Ph), 68.8 (C-6), 54.7 (C-2); MS-ES 818-816 [M+Na]⁺; Anal. Calcd. for C₃₇H₃₉Cl₃NO₁₀P: C, 55.90; H, 4.94; N, 1.76%. Found: C, 55.67; H, 5.18; N, 1.62%.

### 3,6-Di-O-benzyl-4-O-(dibenzyloxyphosphoryl)-2-deoxy-2-(2,2,2-trichloroethoxycarbonylamino)-D-glucopyranosyl trichloroacetimidate (7b)

To a stirred solution of 6b (419 mg; 0.53 mmol) in dry CH₂Cl₂ (6.5 mL) at room temperature was added trichloroacetonitrile (528 μl; 5.3 mmol) and cesium carbonate (86 mg, 0.26 mmol). After stirring for 1 h, the reaction was quenched with a saturated aqueous NaHCO₃ solution (5 mL) and the solution was extracted. The organic layer was washed with brine, dried over MgSO₄ and the solvent removed in vacuo to give 7b (400 mg) as a pale yellow oil which was used in the next step without further purification.

### Allyl-3,4-di-O-benzyl-2-deoxy-2-(2,2,2-trichloroethoxycarbonylamino)-α-D-glucopyranoside (8b)

To a stirred solution of 3b (937 mg, 1.63 mmol) and borane dimethylamine (482 mg, 8.18 mmol) in CH₂Cl₂ (18 mL) at 0° C. was slowly added BF₃:Et₂O (1 mL, 8.18 mmol). After stirring for 45 min, the mixture was stopped by slowly addition of a saturated aqueous NaHCO₃ solution. The organic phase was separated, washed with a saturated solution of NaCl, and dried over MgSO₄. The solvent was evaporated and the residue recrystallized from EtOAc/n-Heptane to provide 8b (757 mg, 81%) as a white crystalline solid. Mp 119.9° C.; [α]_{D}+74 (c 0.59, CHCl₃); νₘₐₓ cm⁻¹ 3312, 2916, 1702, 1538, 1023, 732, 692; ¹H NMR (500 MHz, CDCl₃): δ 7.40-7.26 (m, 10H, Ph), 5.88 (m, 1H, CH═CH₂), 5.30-5.20 (m, 2H, CH═CH₂), 5.07 (d, 1H, J_{2,NH} 10.0 Hz, NH), 4.89 (d, 1H, J_{1,2} 3.5 Hz, H-1), 4.87 (d, 1H, J 11.0 Hz, CH₂CCl₃), 4.87 and 4.75 (AB, 2H, J 11.0 Hz, CH₂Ph), 4.78 and 4.68 (AB, 2H, J 12.0 Hz, CH₂Ph), 4.68 (d, 1H, J 11.0 Hz, CH₂CCl₃), 4.16 (m, 1H, OCH₂CH), 4.02-3.94 (m, 2H, H-2, OCH₂CH), 3.86-3.64 (m, 5H, H-6, H-6′, H-5, H-4, H-3), 1.78 (m, 1H, OH); ¹³C NMR (125.8 MHz, CDCl₃): δ 154.2 (C═O), 138.0 (Cq), 137.8 (Cq), 133.3 (CH═CH₂), 128.5, 128.4, 128.1, 128.0, 127.8, 127.7 (CH arom), 118.0 (CH═CH₂), 96.8 (C-1), 95.4 (CH₂CCl₃), 80.2 (C-3), 78.0 (C-4), 75.2, 75.1, 74.6 (CH₂CCl₃, 2×CH₂Ph), 71.5 (C-5), 68.3 (OCH₂CH), 61.6 (C-6), 55.2 (C-2); MS-ES 598-596 [M+Na]⁺; Anal. Calcd. for C₂₆H₃₀Cl₃NO₇: C, 54.32; H, 5.26; N, 2.44%. Found: C, 54.76; H, 5.53; N, 2.31%.

### Allyl-2-amino-3,4-di-O-benzyl-2-deoxy-α-D-glucopyranoside (9b)

To a stirred solution of 8b (245 mg, 0.43 mmol) in AcOH (6 mL) at room temperature was added zinc powder (430 mg). After stirring overnight, the suspension was filtered over Celite, the solvent removed in vacuo and the residual solvent was coevaporated with toluene three times. The residue was taken up in EtOAc, washed with a saturated aqueous NaHCO₃ solution and brine. The organic phase was separated, dried over MgSO₄ and the solvent removed in vacuo to give 9b (157 mg) as a colorless oil which was used in the next step without further purification. A sample was purified by flash chromatography on silica gel (CH₂Cl₂/Acetone, 10:1->1:1) to provide compound 9b as a white crystalline solid. Mp 85.2° C.; [α]_{D}+98 (c 0.89, CHCl₃); νₘₐₓ cm⁻¹ 3190, 2899, 1664, 1577, 1496, 1452, 1363, 1024, 737, 695; ¹H NMR (500 MHz, CDCl₃): δ 7.50-7.26 (m, 10H, Ph), 5.92 (m, 1H, CH═CH₂), 5.30-5.20 (m, 2H, CH═CH₂), 4.90 (d, 1H, J_{1,2} 3.5 Hz, H-1), 5.01 and 4.73 (AB, 2H, J 11.0 Hz, CH₂Ph), 4.88 and 4.75 (AB, 2H, J 12.0 Hz, CH₂Ph), 4.16 (dd, 1H, J 5.0 Hz, J 12.9 Hz, OCH₂CH), 4.02 (dd, 1H, J 6.0 Hz, J 12.9 Hz, OCH₂CH), 3.85-3.55 (m, 5H, H-6, H-6′, H-5, H-4, H-3), 2.80 (dd, 1H, J_{2,3} 9.4 Hz, H-2); ¹³C NMR (125.8 MHz, CDCl₃): δ 138.6 (Cq), 138.1 (Cq), 133.9 (CH═CH₂), 128.6, 128.5, 127.9, 127.8, 127.7 (CH arom), 117.3 (CH═CH₂), 98.8 (C-1), 83.8, 78.7, 71.9 (C-3, C-4, C-5), 75.6, 74.8 (2×CH₂Ph), 68.3 (OCH₂CH), 61.4 (C-6), 56.0 (C-2); MS-ES 400 [M+H]⁺; Anal. Calcd. for C₂₃H₂₉NO₅: C, 69.15; H, 7.32; N, 3.51%. Found: C, 69.21; H, 7.36; N, 3.25%.

### Allyl-2-[(R)-3-benzyloxytetradecanoylamino]-3,4-di-O-benzyl-2-deoxy-α-D-glucopyranoside (10b)

To a cold solution (−15° C.) of (R)-3-benzyloxytetradecanoic acid (145 mg; 0.43 mmol) [Bull. Chem. Soc. Jpn, 60 (1987), 2197-2204] in THF (5 mL) were added N-methylmorpholine (47 μL; 0.43 mmol) and isobutyl chloroformate (57 μL; 0.43 mmol). The reaction mixture was stirred at −15° C. for 30 min. A solution of compound 9b (157 mg; 0.39 mmol) in THF (5 mL) was added to the reaction mixture. Stirring was continued overnight at room temperature. Water and EtOAc were then added, the organic phase was separated and the aqueous phase was extracted with EtOAc once more. The organic layers were combined, washed with H₂O and brine, and dried over MgSO₄. The solvent was evaporated and the residue was recrystallized from MeOH to give 10b (176 mg, 63% over the 2 steps) as a white crystalline solid. Mp 141.3° C.; [α]_{D}+61 (c 0.31, CHCl₃); νₘₐₓ cm⁻¹ 3297, 2920, 2851, 1637, 1544, 1025, 732, 693; ¹H NMR (500 MHz, CDCl₃): δ 7.40-7.26 (m, 15H, Ph), 6.32 (d, 1H, J_{2,NH} 9.0 Hz, NH), 5.76 (m, 1H, CH═CH₂), 5.23-5.10 (m, 2H, CH═CH₂), 4.82 and 4.65 (AB, 2H, J 11.0 Hz, CH₂Ph), 4.80 (d, 1H, J_{1,2} 4.0 Hz, H-1), 4.73 and 4.57 (AB, 2H, J=11.5 Hz, CH₂Ph), 4.54 and 4.49 (AB, 2H, J 11.0 Hz, CH₂Ph), 4.27 (m, 1H, J_{1,2} 4.0 Hz, H-2), 4.00 (m, 1H, OCH₂CH), 3.85-3.62 (m, 7H, OCH₂CH, H-6, H-6′, H-5, H-4, H-3, H-3″), 2.40 (dd, 1H, J 3.7, 15.1 Hz, H-2″B), 2.28 (dd, 1H, J=7.6, 15.1 Hz, H-2″A), 1.58 (m, 1H, H-4″A), 1.49 (m, 1H, H-4″B), 1.35-1.12 (m, 18H, 9 CH₂), 0.90 (t, 3H, CH₃); ¹³C NMR (125.8 MHz, CDCl₃): δ 171.2 (C═O), 138.3 (Cq), 138.2 (Cq), 137.9 (Cq), 133.5 (CH═CH₂), 128.5, 128.4, 128.3, 128.0, 127.9, 127.8, 127.7, 127.6, 127.5 (CH arom), 117.7 (CH═CH₂), 96.9 (C-1), 80.2 (C-3), 78.0 (C-4), 76.6 (C-3″), 74.7 (CH₂Ph), 74.6 (CH₂Ph), 71.4 (C-5), 71.3 (CH₂Ph), 68.3 (OCH₂CH), 61.8 (C-6), 52.6 (C-2), 41.5 (C-2″), 33.8 (C-4″), 31.9, 29.6, 29.5, 29.4, 29.3, 25.1, 22.7 (CH₂), 14.1 (CH₃); MS-ES 739 [M+Na]⁺; Anal. Calcd. for C₄₄H₆₁NO₇: C, 73.81; H, 8.59; N, 1.96%. Found: C, 73.62; H, 8.57; N, 1.82%.

### Allyl-3,4-di-O-benzyl-6-O-[3,6-di-O-benzyl-4-O-(dibenzyloxyphosphoryl)-2-deoxy-2-(2,2,2-trichloroethoxycarbonylamino)-β-D-glucopyranosyl]-2-[(R)-3-benzyloxytetradecanoylamino]-2-deoxy-α-D-glucopyranoside (11a)

To a stirred solution of 10b (231 mg, 0.32 mmol) and imidate 7b (400 mg, 0.42 mmol) in anhydrous CH₂Cl₂ (9 mL) at −20° C. was added 4 Å molecular sieves. After stirring for 30 min, TMSOTf (12 μL, 64 μmol) was added and stirring continued for additional 1 h. The reaction was filtered over Celite, diluted with EtOAc and neutralized with a saturated aqueous NaHCO₃ solution. The organic phase was separated, washed with a saturated solution of NaCl, dried over MgSO₄. The solvent was evaporated and the residue was recrystallized from MeOH to give 11a (335 mg, 70%) as a white crystalline solid. Mp 145.2° C.; [α]_{D}+37 (c 0.12, CHCl₃); νₘₐₓ cm⁻¹ 3297, 2921, 1713, 1641, 1540, 1453, 1266, 997, 730, 693; ¹H NMR (500 MHz, CDCl₃): δ 7.37-7.14 (m, 35H, Ph), 6.32 (d, 1H, J_{2,NH} 9.0 Hz, NH-2), 5.76 (m, 1H, CH═CH₂), 5.23-5.10 (m, 3H, NH-2′, CH═CH₂), 4.95-4.42 (m, 15H, CH₂CCl₃, 7×CH₂Ph), 4.79 (d, 1H, J_{1,2} 3.5 Hz, H-1), 4.74 (d, 1H, J_{1′,2′} 10.0 Hz, H-1′), 4.46 (t, 1H, J_{3′,4′}=J_{4′,5′} 8.5 Hz, H-4′), 4.32 (m, 1H, J_{1,2} 4.0 Hz, H-2), 4.25 (AB, 1H, CH₂Ph), 4.13 (m, 1H, H-6A), 4.08 (m, 1H, H-3′), 4.04 (m, 1H, OCH₂CH), 3.90-3.60 (m, 9H, OCH₂CH, H-5′, H-6′A, H-6′B, H-6B, H-5, H-4, H-3, H-3″), 3.42 (m, 1H, H-2′), 2.40 (dd, 1H, J 3.7, 15.1 Hz, H-2″B), 2.28 (dd, 1H, J=7.6, 15.1 Hz, H-2″A), 1.58 (m, 1H, H-4″A), 1.49 (m, 1H, H-4″B), 1.35-1.12 (m, 18H, 9CH₂), 0.90 (t, 3H, CH₃); ¹³C NMR (125.8 MHz, CDCl₃): δ 171.0 (C═O), 153.7 (C═O), 138.3 (Cq), 138.2 (Cq), 138.1 (Cq), 137.9 (Cq), 137.7 (Cq), 135.7 (Cq), 135.6 (Cq), 133.6 (CH═CH₂), 128.5, 128.4, 128.3, 128.2, 128.1, 128.0, 127.9, 127.8, 127.7, 127.6, 127.5, 127.4 (CH arom), 117.7 (CH═CH₂), 99.8 (C-1′), 96.9 (C-1), 95.1 (CH₂CCl₃), 80.6 (C-3), 78.6 (C-3′), 78.0 (C-4), 76.6 (C-3″), 76.2 (C-4′), 74.4 (C-5′), 74.7, 74.2, 73.8, 73.3, 71.3 (CH₂CCl₃, 5×CH₂Ph), 70.2 (C-5), 69.6, 69.4 (2×P—OCH₂Ph), 69.0 (C-6 or C-6′), 68.1 (OCH₂CH), 67.7 (C-6 or C-6′), 57.3 (C-2′), 52.6 (C-2), 41.6 (C-2″), 33.8 (C-4″), 31.9, 29.6, 29.5, 29.4, 29.3, 25.1, 22.7 (CH₂), 14.1 (CH₃); MS-ES 1515-1513 [M+Na]; Anal. Calcd. for C₈₁H₉₈Cl₃N₂O₁₆P: C, 65.16; H, 6.62; N, 1.88%. Found: C, 65.31; H, 6.70; N, 1.77%.

### Allyl-6-O-[2-amino-3,6-di-O-benzyl-4-O-(dibenzyloxyphosphoryl)-2-deoxy-β-D-glucopyranosyl]-2-[(R)-3-benzyloxytetradecanoylamino]-3,4-di-O-benzyl-2-deoxy-α-D-glucopyranoside (12d)

To a stirred solution of 11a (310 mg, 0.21 mmol) in AcOH/H₂O 9:1 (5 mL) at room temperature was added zinc-copper couple (260 mg). After stirring 1 h, zinc-copper couple (260 mg) was added and the operation repeated once more. Stirring was continued for another 3H and the suspension was filtered over Celite. The solvent was removed in vacuo and the residual solvent was coevaporated with toluene three times. The residue was taken up in EtOAc, washed with a saturated aqueous NaHCO₃ solution (2×) and brine. The organic phase was separated, dried over MgSO₄ and the solvent removed in vacuo to give 12d (273 mg) as a colorless oil which was used in the next step without further purification. MS-ES 1317 [M+H]⁺, 1339 [M+Na]⁺

### Allyl-3,4-di-O-benzyl-6-O-{3,6-di-O-benzyl-4-O-(dibenzyloxyphosphoryl)-2-deoxy-2-[(R)-3-dodecanoyloxytetradecanoylamino]-β-D-glucopyranosyl}-2-[(R)-3-benzyloxytetradecanoylamino]-2-deoxy-α-D-glucopyranoside (13b)

To a stirred solution of (R)-3-dodecanoyloxytetradecanoic acid [Bull. Chem. Soc. Jpn, 60 (1987), 2205-2214] (115 mg; 0.27 mmol) in THF (4 mL) at −15° C. were added successively N-methylmorpholine (30 μl; 0.27 mmol) and isobutyl chloroformate (35 μL; 0.27 mmol). Stirring was continued for 30 min at −15° C. A solution of crude 12d (273 mg; 0.21 mmol) in THF (4 mL) was then added to the reaction mixture. After stirring overnight at room temperature, the solvent was removed in vacuo and H₂O was added to the residue. The mixture was then extracted with EtOAc, the organic phases was washed successively with a saturated aqueous NaHCO₃ solution, brine and dried over MgSO₄. The solvent was evaporated and the residue was crystallized from MeOH to give 13b (259 mg, 72% over 2 steps) as a white solid. Mp 173° C.; [α]_{D}+30 (c 0.90, CHCl₃); νₘₐₓ cm⁻¹ 3302, 2919, 2850, 1726, 1636, 1544, 1453, 1357, 1266, 998, 730, 694; ¹H NMR (500 MHz, CDCl₃): δ 7.40-7.10 (m, 35H, Ph), 6.31 (d, 1H, J_{2,NH} 9.4 Hz, NH-2), 6.01 (d, 1H, J_{2′,NH′} 7.5 Hz, NH-2′), 5.76 (m, 1H, CH═CH₂), 5.23-5.08 (m, 2H, CH═CH₂), 5.08 (d, 1H, J_{1′,2′} 8.2 Hz, H-1′), 5.05 (m, 1H, H-3′″), 4.95-4.42 (m, 14H, 7×CH₂Ph), 4.79 (d, 1H, J_{1,2} 3.4 Hz, H-1), 4.46 (t, 1H, J_{3′,4′}=J_{4′,5′} 8.7 Hz, H-4′), 4.32 (m, 2H, H-2, H-3′), 4.09 (m, 1H, H-6A), 4.03 (m, 1H, OCH₂CH), 3.90-3.60 (m, 9H, OCH₂CH, H-5′, H-6′A, H-6′B, H-6B, H-5, H-4, H-3, H-3″), 3.46 (m, 1H, H-2′), 2.40 (dd, 1H, J 3.3, 15.1 Hz, H-2″B), 2.34 (dd, 1H, J 7.2, 15.5 Hz, H-2′″B), 2.28 (dd, 1H, J=7.6, 15.1 Hz, H-2″A), 2.21 (dd, 1H, J 5.0, 15.5 Hz, H-2′″A), 2.11 (t, 2H, J 7.5, H-2″″), 1.58 (m, 1H, H-4″A), 1.55-1.36 (m, 3H, H-4″B, 2×H-4′″), 1.35-1.12 (m, 54H, 27 CH₂), 0.90 (m, 9H, 3×CH₃); ¹³C NMR (125.8 MHz, CDCl₃): δ 173.3 (C═O), 171.0 (C═O), 170.0 (C═O), 138.4 (Cq), 138.3 (Cq), 138.2 (Cq), 138.1 (Cq), 137.8 (Cq), 135.7 (Cq), 135.6 (Cq), 133.6 (CH═CH₂), 128.5, 128.4, 128.3, 128.2, 128.1, 128.0, 127.9, 127.8, 127.7, 127.6, 127.5, 127.4 (CH arom), 117.6 (CH═CH₂), 99.2 (C-1′), 96.8 (C-1), 80.5 (C-3), 78.3 (C-3′, C-4), 76.6 (C-3″), 76.0 (C-4′), 74.8, 74.7 (2×CH₂Ph), 74.3 (C-5′), 73.2, 73.1 (2×CH₂Ph), 71.3 (CH₂Ph), 70.5 (C-3′″), 70.3 (C-5), 69.4, 69.3 (2×P—OCH₂Ph), 69.1 (C-6 or C-6′), 68.1 (OCH₂CH), 67.6 (C-6 or C-6′), 56.7 (C-2′), 52.4 (C-2), 41.6-41.4 (C-2″, C-2′″), 34.4, 34.1, 33.8 (C-4″, C-4′″, C-2″″), 31.9, 29.9, 29.6, 29.5, 29.4, 29.3, 29.2, 29.0, 25.1, 25.0, 24.9, 22.7 (CH₂), 14.1 (CH₃); MS-ES 1747 [M+Na]⁺; Anal. Calcd. for C₁₀₄H₁₄₅N₂O₁₇P: C, 72.36; H, 8.47; N, 1.62%. Found: C, 72.52; H, 8.43; N, 1.51%.

### 3,4-Di-O-benzyl-6-O-{3,6-di-O-benzyl-4-O-(dibenzyloxyphosphoryl)-2-deoxy-2-[(R)-3-dodecanoyloxytetradecanoylamino]-β-D-glucopyranosyl}-2-[(R)-3-benzyloxytetradecanoylamino]-2-deoxy-D-glucopyranose (14b)

To a stirred solution of 13b (259 mg; 0.15 mmol) in dry THF (13 mL) at room temperature was added [bis(methyldiphenylphosphine)]-(1,5-cyclooctadiene)Iridium(I) hexafluorophosphate (13 mg). After activation of the iridium catalyst with hydrogen for 1 min (the slightly red solution becomes colorless), the mixture was stirred under nitrogen for 1 h. Iodine (69 mg, 0.27 mmol) and water (13 mL) were added and the reaction mixture was stirred for additional 15 min. To the mixture was added 10% aqueous Na₂S₂O₃ solution and the solution was extracted with EtOAc. The organic layer was washed successively with 10% aqueous Na₂S₂O₃ solution (2×) and brine. The organic phase was dried over MgSO₄, the solvent removed in vacuo and the residue crystallized from CH₃CN to give 14b (165 mg; 65%) as a gray solid. νₘₐₓ cm⁻¹ 3388, 3276, 3062, 2919, 2850, 1726, 1641, 1544, 1453, 1358, 1263, 997, 731, 694; ¹H NMR (500 MHz, CDCl₃) for α-anomer: δ 7.40-7.10 (m, 35H, Ph), 6.39 (d, 1H, J_{2,NH} 9.4 Hz, NH-2), 6.18 (m, 1H, NH-2′), 5.38 (d, 1H, J_{1′,2′} 7.7 Hz, H-1′), 5.12 (m, 1H, J_{1,2} 3.2 Hz, H-1), 5.05 (m, 1H, H-3′″), 4.95-4.42 (m, 14H, 7×CH₂Ph), 4.50 (m, 1H, H-4′), 4.23 (dt, 1H, J_{1,2} 3.2 Hz, J_{2,NH}=J_{2,3} 9.4 Hz, H-2), 4.19 (t, 1H, J_{2′,3′}=J_{3′,4′} 8.9 Hz, H-3′), 4.07 (m, 1H, H-5), 3.96 (d, 1H, J_{6A,6B} 11.4 Hz, H-6A), 3.89-3.80 (m, 2H, H-3″, H-6′A), 3.80-3.65 (m, 4H, H-5′, H-6′B, H-6B, H-3), 3.35 (m, 1H, H-2′), 3.32 (t, 1H, J_{3,4}=J_{4,5} 9.5 Hz, H-4), 2.40-2.20 (m, 4H, 2×H-2″, 2×H-2′″), 2.16 (t, 2H, J 7.5, H-2″″), 1.58 (m, 1H, H-4″A), 1.55-1.36 (m, 3H, H-4″B, 2×H-4′″), 1.35-1.12 (m, 54H, 27 CH₂), 0.90 (m, 9H, 3×CH₃); ¹³C NMR (125.8 MHz, CDCl₃) for α-anomer: δ 173.9 (C═O), 171.4 (C═O), 170.6 (C═O), 138.4 (Cq), 138.3 (Cq), 138.2 (Cq), 138.1 (Cq), 137.8 (Cq), 135.7 (Cq), 135.6 (Cq), 128.5, 128.4, 128.3, 128.2, 128.1, 128.0, 127.9, 127.8, 127.7, 127.6, 127.5, 127.4 (CH arom), 98.8 (C-1′), 91.7 (C-1), 80.6 (C-3), 78.8 (C-3′, C-4), 76.8 (C-3″), 76.2 (C-4′), 74.8 (CH₂Ph), 74.2 (C-5′), 73.6, 73.4, 73.3 (3×CH₂Ph), 71.8 (C-5), 71.3 (CH₂Ph), 70.8 (C-3′″), 69.4, 69.3 (2×P—OCH₂Ph), 69.0 (C-6′), 67.6 (C-6), 57.0 (C-2′), 52.9 (C-2), 41.7 (C-2″, C-2′″), 34.4, 34.1, 33.8 (C-4″, C-4′″, C-2″″), 31.9, 29.9, 29.6, 29.5, 29.4, 29.3, 29.2, 29.0, 25.1, 25.0, 24.9, 22.7 (CH₂), 14.1 (CH₃); MS-ES 1708 [M+Na]⁺; Anal. Calcd. for C₁₀₁H₁₄₁N₂O₁₇P: C, 71.94; H, 8.43; N, 1.66%. Found: C, 71.68; H, 8.35; N, 1.61%.

### 3,4-Di-O-benzyl-6-O-{3,6-di-O-benzyl-4-O-(dibenzyloxyphosphoryl)-2-deoxy-2-[(R)-3-dodecanoyloxytetradecanoylamino]-β-D-glucopyranosyl}-2-[(R)-3-benzyloxytetradecanoylamino]-2-deoxy-α-D-glucopyranosyldibenzyloxyphosphate (15b)

To a stirred solution of 14b (1 g, 0.60 mmol) in THF (90 mL) at −78° C. was added lithium bis(trimethylsilyl)amide solution (1M in THF) (1.9 mL, 1.88 mmol). The mixture was stirred 5 min then tetrabenzyl pyrophosphate (1.3 g, 2.37 mmol) was added. Stirring was continued at −78° C. for 2 h then the solution was neutralized with a saturated aqueous NaHCO₃ solution and diluted with EtOAc. The organic phase was separated, dried over MgSO₄ and the solvent removed in vacuo to give 15b (2 g) as a pale yellow oil which was used in the next step without further purification.

### 2-Deoxy-6-O-[2-deoxy-4-O-(dihydroxyphosphoryl)-2-[(R)-3-dodecanoyloxytetradecanoylamino]-β-D-glucopyranosyl]-2-[(R)-3-hydroxytetradecanoylamino]-α-D-glucopyranosyl dihydrogenophosphate (1b) (OM-174-DP)

Crude compound 15b (2 g) in THF (100 mL) was hydrogenated for 17 h in the presence of 5% Pd—C (1.5 g) at room temperature under hydrogen (6 bars). The mixture was then neutralized with Et₃N (500 μL) and the catalyst was removed by filtration. The filtrate was concentrated in vacuo and the residue was purified by HPLC according to the invention (Method D) to give 1b (as a sodium salt) (342 mg; 48% over 2 steps) as a white lyophilizate. [α]_{D}+14 (c 0.6, H₂O); νₘₐₓ cm⁻¹ 3305, 2918, 2849, 1713, 1648, 1553, 1465, 1376, 1174, 1039, 915, 719, 654; ¹H NMR (500 MHz, CDCl₃/CD₃OD/Pyridine-d₅/DCl 137% 5/2/1/1): δ 5.60 (dd, 1H, J_{1,2} 7.5 Hz, J_{1,P} 2.0 Hz, H-1), 5.27 (m, 1H, H-3′″), 4.73 (d, 1H, J_{1′,2′} 8.5 Hz, H-1′), 4.09 (q, 1H, J_{3,4}=J_{4,5}=J_{4,P} 9.0 Hz, H-4′), 4.05-3.80 (m, 6H, 2×H-6, 2×H-6′, H-4, H-3), 4.00 (m, 1H, H-3″), 3.85 (m, 2H, H-2, H-3′), 3.85 (m, 1H, H-2′), 3.50 (m, 2H, H-5, H-5′), 2.67 (m, 2H, J 6.4 Hz, 2×H-2′″), 2.43 (m, 2H, J 6.1 Hz, 2×H-2″), 2.29 (m, 2H, J 7.3, J 15 Hz, 2×H-2″″), 1.60-1.36 (m, 6H, 2×H-4″, 2×H-4′″, 2×H-3″″), 1.35-1.12 (m, 52H, 26 CH₂), 0.90 (m, 9H, 3×CH₃); ¹³C NMR (125.8 MHz, CDCl₃/CD₃OD/Pyridine-d₅/DCl37% 5/2/1/1): δ 174.9 (C═O), 174.1 (C═O), 172.9 (C═O), 102.4 (C-1′), 94.7 (C-1), 75.3 (C-5′), 73.8 (C-4′), 73.4, 70.4, 70.1, 69.6 (C-3′, C-5, C-4, C-3), 71.9 (C-3′″), 69.6 (C-3″), 69.4 (C-6), 60.8 (C-6′), 56.1 (C-2′), 54.8 (C-2), 44.2 (C-2″), 41.7 (C-2′″), 37.7, 35.1, 34.6 (C-4″, C-4′″, C-2″″), 32.3 (C-12″, C-12′″, C-10″″), 30.1-29.6 (C-6″->C-11″, C-6′″->C-11′″, C-4″″->C-9″″), 25.7, 25.6, 25.2 (C-5″, C-5′″, C-3″″), 23.1 (C-13″, C-13′″, C-11″″), 14.5 (C-14″, C-14′″, C-12″″); MS-ES 1155 [M+Na−2H]⁻, 1133 [M−H]⁻; Anal. Calcd. for C₅₂H₉₇N₂O₂₀P₂ Na₃+H₂O: C, 51.23; H, 8.18; N, 2.30%. Found: C, 51.11; H, 8.46; N, 2.20%.

### 2-Deoxy-6-O-[2-deoxy-4-O-(dihydroxyphosphoryl)-2-[(R)-3-dodecanoyloxytetradecanoylamino]-β-D-glucopyranosyl]-2-[(R)-3-hydroxytetradecanoylamino]-α-D-glucopyranose (16) (OM-174-MP)

Compound 14b (80 mg, 47 μmol) in THF (50 mL) was hydrogenated for 16 h in the presence of 5% Pd—C (25 mg) at room temperature under hydrogen (6 bars). The catalyst was removed by filtration and the filtrate was concentrated in vacuo. The residue was purified by HPLC according to the invention (Method B) to give 16 (as a sodium salt) (25 mg; 50%) as a white lyophilizate. MS-ES 1053 [M−H]⁻

### Propyl-2-Deoxy-6-O-[2-deoxy-4-O-(dihydroxyphosphoryl)-2-[(R)-3-dodecanoyloxytetradecanoylamino]-β-D-glucopyranosyl]-2-[(R)-3-hydroxytetradecanoylamino]-α-D-glucopyranoside (17) (OM-174-MP-PR)

Compound 13b (100 mg, 58 μmol) in THF (100 mL) was hydrogenated for 17 h in the presence of 5% Pd—C (50 mg) at room temperature under hydrogen (6 bars). The mixture was then neutralized with Et₃N (500 μL) and the catalyst was removed by filtration. The filtrate was concentrated in vacuo and the residue was purified by HPLC according to the invention (Method D) to give 17 (as a sodium salt) (28 mg; 44%) as a white lyophilizate. ¹H NMR (500 MHz, CDCl₃/CD₃OD/Pyridine-d₅/DCl 37% 5/2/1/1): δ 5.25 (m, 1H, H-3′″), 4.72 (d, 1H, J_{1,2} 3.6 Hz, H-1), 4.70 (d, 1H, J_{1′,2′} 9.9 Hz, H-1′), 4.16 (q, 1H, J_{3,4}=J_{4,5}=J_{4,P} 9.2 Hz, H-4′), 4.01 (dd, 1H, J_{6A,6B} 9.3 Hz, H-6A), 3.95 (m, 1H, H-3″), 3.91 (t, 1H, J_{3′,4′}=J_{3′,2′} 10.0 Hz, H-3′), 3.89 (ddd, 1H, J_{1,2} 3.6 Hz, J_{3,2} 10.5 Hz, J_{NH,2} 8.0 Hz, H-2), 3.86-3.81 (m, 4H, 2×H-6′, H-6B, H-2′), 3.78 (dd, 1H, J_{3,4} 9.0 Hz, J_{3,2} 10.5 Hz, H-3), 3.64 (m, 1H, H-5), 3.56 (t, 1H, J_{3,4}=J_{4,5} 8.8 Hz, H-4), 3.54 (m, 1H, OCH₂CH), 3.50 (m, 1H, H-5′), 3.27 (m, 1H, OCH₂CH), 2.63 (m, 2H, J 6.2 Hz, 2×H-2′″), 2.48 (dd, 1H, J_{2″,3″} 3.4 Hz, J_{2″,2″} 14.8 Hz, H-2″), 2.39 (dd, 1H, J_{2″,3″} 8.5 Hz, J_{2″,2″} 14.8 Hz, H-2″), 2.28 (m, 2H, J 7.3, J 15 Hz, 2×H-2″″), 1.70-1.36 (m, 6H, 2×H-4″, 2×H-4′″, 2×H-3″″), 1.55 (m, 2H, OCH₂CH₂), 1.35-1.12 (m, 52H, 26 CH₂), 0.88 (m, 12H, 4×CH₃); ¹³C NMR (125.8 MHz, CDCl₃/CD₃OD/Pyridine-d₅/DCl 37% 5/2/1/1): δ 174.7 (C═O), 174.0 (C═O), 172.5 (C═O), 101.9 (C-1′), 97.5 (C-1), 75.3 (C-5′), 75.0 (C-4′), 73.7 (C-3′), 71.7 (C-3′″), 71.6, 71.4, 70.6 (C-5, C-4, C-3), 70.1 (OCH₂CH), 69.1 (C-3″), 69.0 (C-6), 60.8 (C-6′), 55.9 (C-2′), 54.4 (C-2), 43.4 (C-2″), 41.5 (C-2′″), 37.4, 35.0, 34.5 (C-4″, C-4′″, C-2″″), 32.3 (C-12″, C-12′″, C-10″″), 30.0-29.6 (C-6″->C-11″, C-6′″->C-11′″, C-4″″->C-9″″), 26.1, 25.6, 25.5 (C-5″, C-5′″, C-3″″), 23.0, 22.9 (OCH₂CH₂CH₃, C-13″, C-13′″, C-11″″), 14.5 (C-14″, C-14′″, C-12″″), 10.9 (OCH₂CH₂CH₃); MS-ESI 1141 [M−H+2Na]⁺, HRMS-ESI calcd for C₅₅H₁₀₄N₂O₁₇Na₂P [M−H+2Na]⁺1141.6868, found 1141.6879.

### 2,3-Dihydroxypropyl-3,4-di-O-benzyl-6-O-{3,6-di-O-benzyl-4-O-(dibenzyloxyphosphoryl)-2-deoxy-2-[(R)-3-dodecanoyloxytetradecanoylamino]-β-D-glucopyranosyl}-2-[(R)-3-benzyloxytetradecanoylamino]-2-deoxy-α-D-glucopyranoside (18)

To a stirred solution of 13b (500 mg; 0.29 mmol) in a mixture THF/t-BuOH/H₂O 10:10:1 (15 mL) at room temperature were successively added 4-methylmorpholine N-oxide (NMO) (156 mg; 1.16 mmol) and OsO₄ in 2-propanol (2.5%; 580 μL; 58 μmol). After 4 h, saturated aqueous Na₂S₂O₃ solution was added, and the mixture was extracted with EtOAc. The organic phase was washed with saturated aqueous Na₂S₂O₃ solution (2×), brine and dried over MgSO₄. The solvent was evaporated and the residue was crystallized from EtOH to give 18 (300 mg, 59%) as a white solid. νₘₐₓ cm⁻¹ 3300, 2919, 2850, 1646, 1543, 1453, 1358, 1266, 998, 730, 694; ¹H NMR (500 MHz, CDCl₃): δ 7.40-7.10 (m, 35H, Ph), 6.54 (m, 1H, NH-2′), 6.38 (m, 1H, NH-2), 5.08 (m, 0.5H, H-3′″), 5.03 (m, 0.5H, H-3′″), 4.95-4.42 (m, 14H, 7×CH₂Ph), 4.88 (m, 1H, H-1′), 4.72 (m, 1H, H-1), 4.52 (m, 1H, H-4′), 4.27 (m, 2H, H-2, H-3′), 4.18-3.30 (m, 15H, OCH₂CH, CH(OH), CH(OH, H-2′, H-5′, 2×H-6′, 2×H-6, H-5, H-4, H-3, H-3″), 2.50-2.45 (m, 1H, H-2′″), 2.45-2.35 (m, 1H, H-2″), 2.30-2.20 (m, 2H, H-2″, H-2′″), 2.15 (m, 1H, H-2″″), 1.65-1.45 (m, 6H, 2×H-4″, 2×H-4′″, 2×H-3″″), 1.35-1.12 (m, 52H, 26 CH₂), 0.90 (m, 9H, 3×CH₃); ¹³C NMR (125.8 MHz, CDCl₃): δ 173.8 (C═O), 173.7 (C═O), 171.1 (C═O), 170.6 (C═O), 170.4 (C═O), 138.2-138.02 (Cq), 137.7 (Cq), 137.6 (Cq), 135.7 (Cq), 135.6 (Cq), 128.4-127.4 (CH arom), 99.4 (C-1′), 99.2 (C-1′), 98.5 (C-1), 98.4 (C-1), 80.6 (C-3), 80.4 (C-3), 78.7, 78.4 (C-3′, C-4), 76.8, 76.7 (C-3″), 75.6, 75.5, 75.4, 75.3 (C-4′), 74.8, 74.7 (2×CH₂Ph), 74.1 (C-5′), 73.2, 72.6, 72.5, 72.0 (2×CH₂Ph), 71.3, 71.2 (CH₂Ph), 70.8-70.5 (C-3′″, C-5, CH(OH)), 69.4, 69.3 (2×P—OCH₂Ph), 68.8, 68.7, 68.6, 68.1 (C-6, (OCH₂CH), C-6′), 63.7, 63.6 (CH(OH), 56.2, 56.1 (C-2′), 52.6, 52.5 (C-2), 41.8-41.4 (C-2″, C-2′″), 34.4, 34.0, 33.7 (C-4″, C-4′″, C-2″″), 31.9 (C-12″, C-12′″, C-10″″), 29.8-29.1 (C-6″->C-11″, C-6′″->C-11′″, C-4″″->C-9″″), 25.2, 25.1, 25.0, 24.9 (C-5″, C-5′″, C-3″″), 22.6 (C-13″, C-13′″, C-11″″), 14.1 (C-14″, C-14′″, C-12″″); MS-ES 1782 [M+Na]⁺; Anal. Calcd. for C₁₀₄H₁₄₇N₂O₁₉P: C, 70.96; H, 8.42; N, 1.59%. Found: C, 70.44; H, 8.36; N, 1.47%.

### 2,3-Dihydroxypropyl-2-Deoxy-6-O-[2-deoxy-4-O-(dihydroxyphosphoryl)-2-[(R)-3-dodecanoyloxytetradecanoylamino]-β-D-glucopyranosyl]-2-[(R)-3-hydroxytetradecanoylamino]-α-D-glucopyranoside (19) (OM-174-MP-PD)

Compound 18 (113 mg, 64 μmol) in THF (100 mL) was hydrogenated for 19 h in the presence of 5% Pd—C (50 mg) at room temperature under hydrogen (6 bars). The mixture was then neutralized with Et₃N (500 μL) and the catalyst was removed by filtration. The filtrate was concentrated in vacuo and the residue was purified by HPLC according to the invention (Method D) to give 19 (as a sodium salt) (26 mg; 36%) as a white lyophilizate. MS-ESI 1173 [M−H+2Na]⁺, HRMS-ESI calcd for C₅₅H₁₀₄N₂O₁₉Na₂P [M−H+2Na]⁺1173.6766, found 1173.6763.

### 3,4-Di-O-benzyl-6-O-{3,6-di-O-benzyl-4-O-(dibenzyloxyphosphoryl)-2-deoxy-2-[(R)-3-dodecanoyloxytetradecanoylamino]-β-D-glucopyranosyl}-2-[(R)-3-benzyloxytetradecanoylamino]-2-deoxy-D-glucopyranosyl trichloroacetimidate (24b)

To a stirred solution of 14b (260 mg; 150 μmol) in dry CH₂Cl₂ (5 mL) at room temperature was added trichloroacetonitrile (155 μl; 1.54 mmol) and potassium carbonate (11 mg, 77 μmol). After stirring for 1 h, the reaction was quenched with a saturated aqueous NaHCO₃ solution (2 mL) and the solution was extracted. The organic layer was washed with brine, dried over MgSO₄ and the solvent removed in vacuo to give 24b (280 mg) as a pale yellow oil which was used in the next step without further purification.

### (6-Benzyloxycarbonylaminohexyl)-3,4-di-O-benzyl-6-O-{3,6-di-O-benzyl-4-β-(dibenzyloxyphosphoryl)-2-deoxy-2-[(R)-3-dodecanoyloxytetradecanoylamino]-β-D-glucopyranosyl}-2-[(R)-3-benzyloxytetradecanoylamino]-2-deoxy-β-D-glucopyranoside (25)

To a stirred solution of commercially available 6-benzyloxycarbonylamino-1-hexanol (43 mg, 0.17 mmol) and crude imidate 24b (280 mg) in anhydrous CH₂Cl₂ (5 mL) at −20° C. was added 4 Å molecular sieves. After stirring for 30 min, TMSOTf (6 μL, 31 μmol) was added and stirring continued for additional 2 h. The mixture was slowly warmed up to room temperature and stirred overnight. The reaction was filtered over Celite, diluted with EtOAc and neutralized with a saturated aqueous NaHCO₃ solution. The organic phase was separated, washed with a saturated solution of NaCl, dried over MgSO₄. The solvent was evaporated and the residue was recrystallized from MeOH to give 25 (174 mg, 59% over 2 steps) as a white crystalline solid. MS-ES 1942 [M+Na]

### 6-Aminohexyl-2-deoxy-6-O-[2-deoxy-4-O-(dihydroxyphosphoryl)-2-[(R)-3-dodecanoyloxytetradecanoylamino]-β-D-glucopyranosyl]-2-[(R)-3-hydroxytetradecanoylamino]-β-D-glucopyranoside (26) (OM-174-MP-AC)

Compound 25 (102 mg, 53 μmol) in a mixture AcOH/2-propanol/CH₂Cl₂ 3:3:1 (7 mL) was hydrogenated for 24 h in the presence of 10% Pd—C (50 mg) at room temperature under an atmospheric pressure. The catalyst was removed by filtration and the filtrate was concentrated in vacuo. The residue was purified by HPLC according to the invention (Method D) to give 26 (as a sodium salt) (17 mg; 28%) as a white lyophilizate. MS-ES 1153 [M−H]⁻.

### Tetradecyl-3,4-di-O-benzyl-6-O-{3,6-di-O-benzyl-4-O-(dibenzyloxyphosphoryl)-2-deoxy-2-[(R)-3-dodecanoyloxytetradecanoylamino]-β-D-glucopyranosyl}-2-[(R)-3-benzyloxytetradecanoylamino]-2-deoxy-β-D-glucopyranoside (41b)

To a stirred solution of commercially available tetradecanol (14 mg, 65 μmol) and crude imidate 24 b (98 mg) in anhydrous CH₂Cl₂ (2 mL) at −20° C. was added 4 Å molecular sieves. After stirring for 30 min, TMSOTf (2 μL, 12 μmol) was added and stirring continued for additional 2 h. The mixture was slowly warmed up to room temperature and stirred overnight. The reaction was filtered over Celite, diluted with EtOAc and neutralized with a saturated aqueous NaHCO₃ solution. The organic phase was separated, washed with a saturated solution of NaCl, dried over MgSO₄. The solvent was evaporated and the residue was recrystallized from MeOH to give 41b (58 mg, 52%) as a white crystalline solid. ¹H NMR (500 MHz, CDCl₃): δ 7.37-7.11 (m, 35H, Ph), 6.49 (d, 1H, J_{NH,2} 8.0 Hz, NH-2), 6.21 (d, 1H, J_{NH,2′} 7.5 Hz, NH-2′), 5.11 (m, 1H, H-3′″), 5.02 (d, 1H, J_{1′,2′} 8.0 Hz, H-1′), 4.95-4.40 (m, 14H, 7×CH₂Ph), 4.61 (d, 1H, H-1), 4.51 (m, 1H, H-4′), 4.31 (t, 1H, J_{3′,4′}=J_{3′,2′} 9.5 Hz, H-3′), 4.09 (m, 1H, H-6), 3.96 (m, 1H, H-3), 3.84 (m, 1H, H-6′), 3.79 (m, 1H, OCH₂CH), 3.79-3.65 (m, 5H, H-6′, H-6, H-5′, H-5, H-3″), 3.56-3.47 (m, 2H, H-4, H-2′), 3.44 (m, 1H, H-2), 3.36 (m, 1H, OCH₂CH), 2.42-2.23 (m, 4H, 2×H-2″, 2×H-2′″), 2.12 (t, 2H, J 7.2 Hz, 2×H-2″″), 1.65-1.45 (m, 8H, 2×H-4″, 2×H-4′″, 2×H-3″″, OCH₂CH₂), 1.35-1.12 (m, 74H, 37 CH₂), 0.90 (m, 12H, 4×CH₃); ¹³C NMR (125.8 MHz, CDCl₃): δ 173.4 (C═O), 171.3 (C═O), 170.1 (C═O), 138.2-138.0 (Cq), 135.7-137.6 (Cq), 128.5-127.4 (CH arom), 99.9 (C-1), 99.2 (C-1′), 80.9 (C-3), 78.5 (C-4), 78.3 (C-3′), 76.3 (C-3″), 75.7 (C-4′), 74.6, 74.9 (2×CH₂Ph), 74.2-74.1 (C-5′, C-5), 73.2, 73.0 (2×CH₂Ph), 71.0 (CH₂Ph), 70.6 (C-3′″), 69.6 ((OCH₂CH)), 69.4-69.0 (2×P—OCH₂Ph, C-6′), 67.3 (C-6), 56.6 (C-2), 56.0 (C-2′), 41.4-41.2 (C-2″, C-2′″), 34.4, 34.1, 33.6 (C-4″, C-4′″, C-2″″), 31.9 (C-12″, C-12′″, C-10″″, C-12″″), 29.7-29.1 (C-6″->C-11″, C-6′″->C-11′″, C-4″″->C-9″″, C-3″″″->C-11′″″), 26.1, 25.2, 25.0 (C-5″, C-5′″, C-3″″), 22.7 (C-13″, C-13′″, C-11″″, C-13′″″), 14.1 (C-14″, C-14′″, C-12″″, C-14′″″); MS-ES 1905 [M+Na]⁺.

### Tetradecyl-2-Deoxy-6-O-[2-deoxy-4-O-(dihydroxyphosphoryl)-2-[(R)-3-dodecanoyloxytetradecanoylamino]-β-D-glucopyranosyl]-2-[(R)-3-hydroxytetradecanoylamino]-β-D-glucopyranoside (41c) (OM-174-MP-TE)

Compound 41b (55 mg, 29 μmol) in THF (200 mL) was hydrogenated for 17 h in the presence of 5% Pd—C (20 mg) at room temperature under hydrogen (6 bars). The catalyst was removed by filtration and the filtrate was concentrated in vacuo. The residue was purified by HPLC according to the invention (Method B) to give 41c (as a sodium salt) (10 mg; 27%) as a white lyophilizate. MS-ES 1273 [M+Na]⁺, 1251 [M+H]⁺.

### Formylmethyl-3,4-di-O-benzyl-6-O-{3,6-di-O-benzyl-4-O-(dibenzyloxyphosphoryl)-2-deoxy-2-[(R)-3-dodecanoyloxytetradecanoylamino]-β-D-glucopyranosyl}-2-[(R)-3-benzyloxytetradecanoylamino]-2-deoxy-α-D-glucopyranoside (32c)

To a stirred solution of 18 (300 mg; 0.17 mmol) in THF/H₂O 4:1 (5 mL) at room temperature was added sodium periodate (182 mg; 0.85 mmol). After stirring overnight, the reaction was diluted with EtOAc and the mixture washed with a saturated aqueous NaHCO₃ solution. The organic layer was washed with brine, dried over MgSO₄ and the solvent removed in vacuo. Flash chromatography of the residue on silica gel (Petroleum ether/EtOAc, 2:3) provided compound 32c (250 mg; 85%) as a colorless oil. [α]_{D}+26 (c 0.90, CHCl₃); ¹H NMR (500 MHz, CDCl₃): δ 9.42 (1H, s, CHO), 7.37-7.10 (m, 35H, Ph), 6.55 (d, 1H, J_{NH,2} 9.2 Hz, NH-2), 6.10 (d, 1H, J_{NH,2′} 7.7 Hz, NH-2′), 5.07 (m, 1H, H-3′″), 4.95 (d, 1H, J_{1′,2′} 7.6 Hz, H-1′), 4.94-4.40 (m, 15H, 7×CH₂Ph, H-4′), 4.70 (d, 1H, J_{1,2} 3.6 Hz, H-1), 4.35-4.25 (m, 2H, H-2, H-3′), 4.05 (d, 1H, H-6), 3.95-3.75 (m, 5H, H-5, OCH₂CHO, H-6′, H-3″), 3.75-3.65 (m, 3H, H-6′, H-5′, H-3), 3.60 (m, 1H, H-6), 3.55-3.40 (m, 2H, H-4, H-2′), 2.50-2.15 (m, 4H, 2×H-2″, 2×H-2′″), 2.13 (t, 2H, J=7.5 Hz, 2×H-2″″), 1.70-1.45 (m, 6H, 2×H-4″, 2×H-4′″, 2×H-3″″), 1.35-1.12 (m, 52H, 26 CH₂), 0.90 (m, 9H, 3×CH₃); ¹³C NMR (125.8 MHz, CDCl₃): δ 199.26 (CHO), 173.4 (C═O), 171.3 (C═O), 170.2 (C═O), 138.2-138.0 (Cq), 137.6 (Cq), 135.7 (Cq), 135.6 (Cq), 128.7-127.3 (CH arom), 99.5 (C-1′), 98.4 (C-1), 80.2 (C-3), 78.3 (C-4, C-3′), 76.7 (C-3″), 75.9 (C-4′), 74.8, 74.7 (2×CH₂Ph), 74.2 (C-5′), 73.2, 73.1, 73.0 (2×CH₂Ph, OCH₂CHO), 71.2 (CH₂Ph, C-5), 70.6 (C-3′″), 69.3, 69.2 (2×P—OCH₂Ph), 69.0 (C-6′), 68.1 (C-6), 56.6 (C-2′), 52.3 (C-2), 41.4-41.2 (C-2″, C-2′″), 34.4, 34.0, 33.8 (C-4″, C-4′″, C-2″″), 31.9 (C-12″, C-12′″, C-10″″), 29.6-29.1 (C-6″->C-11″, C-6′″->C-11′″, C-4″″->C-9″″), 25.2, 25.1, 24.9 (C-5″, C-5′″, C-3″″), 22.7 (C-13″, C-13′″, C-11″″), 14.1 (C-14″, C-14′″, C-12″″); MS-ES 1750 [M+Na].

### 2-Hydroxyethyl-3,4-di-O-benzyl-6-O-{3,6-di-O-benzyl-4-O-(dibenzyloxyphosphoryl)-2-deoxy-2-[(R)-3-dodecanoyloxytetradecanoylamino]-β-D-glucopyranosyl}-2-[(R)-3-benzyloxytetradecanoylamino]-2-deoxy-α-D-glucopyranoside (32b)

To a stirred solution of 32c (119 mg; 69 μmol) in EtOH/CH₂Cl₂ 4:1 (5 mL) at 0° C. was added sodium borohydride (3 mg; 75 μmol). After stirring for 10 min, the reaction was quenched with a saturated aqueous NH₄Cl solution and diluted with CH₂Cl₂. The organic layer was extracted, washed with brine, dried over MgSO₄. The solvent was evaporated and the residue was recrystallized from EtOH to give 32b (100 mg, 84%) as a white crystalline solid. Mp 175° C. (EtOH); [α]_{D}+27 (c 0.56, CHCl₃); νₘₐₓ cm⁻¹ 3301, 2920, 2850, 1724, 1649, 1543, 1453, 1358, 1264, 1008, 731, 694; ¹H NMR (500 MHz, CDCl₃): δ 7.40-7.10 (m, 35H, Ph), 6.44 (d, 1H, J_{NH,2′} 7.0 Hz, NH-2′), 6.40 (d, 1H, J_{NH,2} 10.1 Hz, NH-2), 5.14 (m, 1H, H-3′″), 5.00-4.42 (m, 14H, 7×CH₂Ph), 4.88 (m, 1H, J_{1′,2′} 8.72 Hz, H-1′), 4.73 (d, 1H, J_{1,2} 3.6 Hz, H-1), 4.53 (m, 1H, H-4′), 4.47 (m, 1H, H-3′), 4.29 (ddd, 1H, J_{NH,2}=J_{2,3} 10.1 Hz, J_{1,2} 3.6 Hz, H-2), 4.15 (d, 1H, H-6), 3.99 (m, 1H, H-5), 3.86 (m, 2H, H-6′, H-3″), 3.77-3.60 (m, 2H, H-6′, H-5′), 3.66 (t, 1H, J_{3,4}=J_{2,3} 10.1 Hz, H-3), 3.60-3.33 (m, 7H, H-6, H-4, H-2′, OCH₂CH₂, OCH₂CH₂), 2.42 (m, 1H, H-2′″), 2.28 (m, 3H, 2×H-2″, H-2′″), 2.15 (t, 2H, J 7.5 Hz, 2×H-2″″), 1.70-1.45 (m, 6H, 2×H-4″, 2×H-4′″, 2×H-3″″), 1.35-1.12 (m, 52H, 26 CH₂), 0.90 (m, 9H, 3×CH₃); ¹³C NMR (125.8 MHz, CDCl₃): δ 173.3 (C═O), 171.0 (C═O), 170.5 (C═O), 138.2-138.0 (Cq), 137.6 (Cq), 135.7 (Cq), 135.6 (Cq), 128.7-127.3 (CH arom), 98.9 (C-1′), 98.5 (C-1), 80.3 (C-3), 78.6 (C-4), 78.1 (C-3′), 76.7 (C-3″), 75.9 (C-4′), 74.8, 74.7 (2×CH₂Ph), 74.0 (C-5′), 73.2, 73.1 (2×CH₂Ph), 72.3 (CH₂OH), 71.2 (CH₂Ph), 70.8 (C-5), 70.6 (C-3′″), 69.3, 69.2 (2×P—OCH₂Ph), 68.8 (C-6′), 68.2 (C-6), 61.8 (OCH₂CH₂), 57.1 (C-2′), 52.5 (C-2), 41.4-41.2 (C-2″, C-2′″), 34.4, 34.0, 33.7 (C-4″, C-4′″, C-2″″), 31.8 (C-12″, C-12′″, C-10″″), 29.6-29.0 (C-6″->C-11″, C-6′″->C-11′″, C-4″″->C-9″″), 25.1, 25.0, 24.9 (C-5″, C-5′″, C-3″″), 22.6 (C-13″, C-13′″, C-11″″), 14.0 (C-14″, C-14′″, C-12″″); MS-ES 1753 [M+Na]⁺.

### 2-Hydroxyethyl-2-Deoxy-6-O-[2-deoxy-4-O-(dihydroxyphosphoryl)-2-[(R)-3-dodecanoyloxytetradecanoylamino]-β-D-glucopyranosyl]-2-[(R)-3-hydroxytetradecanoylamino]-α-D-glucopyranoside (32) (OM-174-MP-EO)

Compound 32b (160 mg, 92 μmol) in THF (100 mL) was hydrogenated for 17 h in the presence of 5% Pd—C (50 mg) at room temperature under hydrogen (6 bars). The mixture was then neutralized with Et₃N (500 μL) and the catalyst was removed by filtration. The filtrate was concentrated in vacuo and the residue was purified by HPLC according to the invention (Method D) to give 32 (as a sodium salt) (50 mg; 49%) as a white lyophilizate. MS-ESI 1143 [M−H+2Na]⁺, HRMS-ESI calcd for C₅₄H₁₀₂N₂O₁₈Na₂P [M−H+2Na]⁺1143.6660, found 1143.6659.

### 2-(Dibenzyloxyphosphoryloxy)ethyl-3,4-di-O-benzyl-6-O-{3,6-di-O-benzyl-4-O-(dibenzyloxyphosphoryl)-2-deoxy-2-[(R)-3-dodecanoyloxytetradecanoylamino]-β-D-glucopyranosyl}-2-[(R)-3-benzyloxytetradecanoylamino]-2-deoxy-α-D-glucopyranoside (33b)

To a stirred solution of 32b (125 mg, 72 μmol) and a commercially available solution of 1H-tetrazole in CH₃CN (˜0.45 M) (321 μL, 0.14 mmol) in CH₂Cl₂ (5 mL) at room temperature was added dibenzyl dimethylphosphoramidite (29 μl; 0.11 mmol). Stirring was continued at room temperature for 10 min and the solution was then cooled down to −20° C. A solution of mCPBA (57-86%, 46 mg; 0.27 mmol) in CH₂Cl₂ (3 mL) was added and the solution was stirred for 30 min at −20° C. 10% Aqueous sodium thiosulfate (5 mL) was added and the mixture was stirred for 10 min, then diluted with EtOAc and the organic phase was separated. The organic layer was washed successively with 10% aqueous Na₂S₂O₃ solution (3×), saturated aqueous NaHCO₃ solution (2×), N HCl solution (1×) and brine. The organic phase was dried over MgSO₄ and the solvent removed in vacuo. Flash chromatography of the residue on silica gel (Petroleum ether/EtOAc, 1:1 to 1:2) provided compound 33b (130 mg; 90%) as a colorless oil. ¹H NMR (500 MHz, CDCl₃): δ 7.40-7.10 (m, 45H, Ph), 7.07 (d, 1H, J_{NH,2} 9.4 Hz, NH-2), 6.56 (d, 1H, J_{NH,2′} 7.4 Hz, NH-2′), 5.09 (m, 1H, H-3′″), 5.05 (m, 1H, J_{1′,2′} 7.8 Hz, H-1′), 5.00-4.36 (m, 18H, 9×CH₂Ph), 4.66 (d, 1H, J_{1,2} 3.2 Hz, H-1), 4.50 (m, 1H, H-4′), 4.43 (m, 1H, H-3′), 4.33 (ddd, 1H, J_{NH,2}=J_{2,3} 9.4 Hz, J_{1,2} 3.2 Hz, H-2), 4.10 (d, 1H, J_{6,6} 10.0 Hz, H-6), 4.05 (m, 1H, OCH₂CH₂OP), 3.93 (m, 1H, OCH₂CH₂OP), 3.88 (m, 1H, H-3″), 3.83 (d, 1H, J_{6′,6′} 10.0 Hz, H-6′), 3.80 (m, 1H, H-5), 3.73-3.63 (m, 3H, H-5′, H-3, OCH₂CH₂OP), 3.62 (dd, 1H, H-6), 3.51 (t, 1H, J_{3,4}=J_{4,5} 9.5 Hz, H-4), 3.38 (ddd, 1H, J_{NH,2′} 7.4 Hz, J_{1′,2′} 7.8 Hz, J_{2′,3′} 8.8 Hz, H-2′), 3.31 (m, 1H, OCH₂CH₂OP), 2.44 (dd, 1H, J_{2″,3″} 7.5 Hz, J_{2″,2″} 14.7 Hz, H-2″), 2.36 (m, 2H, H-2′″, H-2″), 2.23 (dd, 1H, J_{2″′,3′″} 5.3 Hz, J_{2′″,2′″} 15.2 Hz, H-2′″), 2.10 (t, 2H, J 7.5 Hz, 2×H-2″″), 1.60-1.40 (m, 6H, 2×H-4″, 2×H-4′″, 2×H-3″″), 1.35-1.05 (m, 52H, 26 CH₂), 0.88 (m, 9H, 3×CH₃); ¹³C NMR (125.8 MHz, CDCl₃): δ 173.3 (C═O), 171.5 (C═O), 170.3 (C═O), 138.7 (Cq), 138.5 (Cq), 138.3 (Cq), 138.2 (Cq), 138.0 (Cq), 135.8 (Cq), 135.7 (Cq), 135.6 (Cq), 135.5 (Cq), 135.4 (Cq), 128.6-127.8 (CH arom), 99.2 (C-1′), 98.7 (C-1), 80.8 (C-3), 78.1 (C-4, C-3′), 76.8 (C-3″), 76.0 (C-4′), 74.9, 74.8 (2×CH₂Ph), 74.2 (C-5′), 73.2, 73.1 (2×CH₂Ph), 71.4 (CH₂Ph), 70.7-70.6 (C-5, C-3′″), 69.5-69.3 (4×P—OCH₂Ph), 69.1 (C-6), 68.0 (C-6′), 67.2 (OCH₂CH₂O—P), 66.6 (OCH₂CH₂O—P), 57.0 (C-2′), 52.5 (C-2), 41.6-41.3 (C-2″, C-2′″), 34.4, 34.2, 34.0 (C-4″, C-4′″, C-2″″), 31.9 (C-12″, C-12′″, C-10″″), 29.6-29.1 (C-6″->C-11″, C-6′″->C-11′″, C-4″″->C-9″″), 25.2, 25.0, 24.9 (C-5″, C-5′″, C-3″″), 22.7 (C-13″, C-13′″, C-11″″), 14.1 (C-14″, C-14′″, C-12″″).

### 2-(Phosphonooxy)ethyl-2-Deoxy-6-O-[2-deoxy-4-O-(dihydroxyphosphoryl)-2-[(R)-3-dodecanoyloxytetradecanoylamino]-β-D-glucopyranosyl]-2-[(R)-3-hydroxytetradecanoylamino]-α-D-glucopyranoside (33) (OM-174-MP-EP)

Compound 33b (107 mg, 54 mmol) in THF (80 mL) was hydrogenated for 17 h in the presence of 5% Pd—C (70 mg) at room temperature under hydrogen (6 bars). The mixture was then neutralized with Et₃N (500 μL) and the catalyst was removed by filtration. The filtrate was concentrated in vacuo and the residue was purified by HPLC according to the invention (Method D) to give 33 (as a sodium salt) (35 mg; 55%) as a white lyophilizate. MS-ESI 1245 [M−2H+3Na]⁺, HRMS-ESI calcd for C₅₄H₁₀₂N₂O₂₁Na₃P₂[M−2H+3Na]⁺1245.6143, found 1245.6136.

### 2-Carboxymethyl-3,4-di-O-benzyl-6-O-{3,6-di-O-benzyl-4-O-(dibenzyloxyphosphoryl)-2-deoxy-2-[(R)-3-dodecanoyloxytetradecanoylamino]-β-D-glucopyranosyl}-2-[(R)-3-benzyloxytetradecanoylamino]-2-deoxy-α-D-glucopyranoside (35d)

To a stirred solution of 32c (100 mg; 58 μmol), NaH₂PO₄.H₂O (8 mg, 58 μmol), 2-methyl-2-butene (28 μL, 260 μmol) in THF/H₂O 4:1 (5 mL) at room temperature was added sodium chlorite (20 mg; 173 μmol). After stirring for 6 h, the reaction was quenched with a 1M HCl solution (1 mL) and diluted with CH₂Cl₂. The organic layer was extracted, washed with brine, dried over MgSO₄ and the solvent removed in vacuo. Flash chromatography of the residue on silica gel (CH₂Cl₂/Acetone, 5:1+1% AcOH) provided compound 35d (67 mg; 66%) as a white solid. MS-ES 1766 [M+Na]⁺.

### 2-Carboxymethyl-2-Deoxy-6-O-[2-deoxy-4-O-(dihydroxyphosphoryl)-2-[(R)-3-dodecanoyloxytetradecanoylamino]-β-D-glucopyranosyl]-2-[(R)-3-hydroxytetradecanoylamino]-α-D-glucopyranoside (35c) (OM-174-MP-CM)

Compound 35d (67 mg, 38 μmol) in THF (20 mL) was hydrogenated for 17 h in the presence of 5% Pd—C (25 mg) at room temperature under hydrogen (6 bars). The mixture was then neutralized with Et₃N (500 μL) and the catalyst was removed by filtration. The filtrate was concentrated in vacuo and the residue was purified by HPLC according to the invention (Method D) to give 35c (as a sodium salt) (25 mg; 58%) as a white lyophilizate. MS-ESI 1179 [M−2H+3Na]⁺, HRMS-ESI calcd for C₅₄H₉₉N₂O₁₉Na₃P [M−2H+3Na]⁺1179.6273, found 1179.6275.

### Biological Activity

### 1. Treatment Method A

The products were dissolved in a THF-water mixture (1:1 vol./vol.). The treatment was run by preparative reverse phase HPLC under the following conditions:

### Column: VYDAC C18, 22×250 mm, 10 μm, 300 Å

### Mobile Phase:

A: Acetonitrile-water (1:1, vol./vol.), 5 mM Tetrabutylammonium phosphate monobasic
B: 2-propanol-water (9:1, vol./vol.), 5 mM Tetrabutylammonium phosphate monobasic
Flow rate: 20 ml/min.

### Elution:

| Time (min) | % mobile phase (B) |
|---|---|
| 0 | 10 |
| 50 | 100 |
| 55 | 10 |
| 60 | 10 |

Detection: UV, 210 nm (wavelength)

Fractions containing the compounds in the form of a tetrabutylammonium salt were collected and concentrated by adsorption on a HPLC, VYDAC C18, 22×250 mm, 10 μm, 300 Å. The sodium salt of the compound is obtained through washing with a 200 mM sodium phosphate monobasic solution in water, pH 4.2+2-propanol (9:1, v/v) (5 volumes). After removal of the excess of sodium phosphate monobasic by running through 5 volumes of water+2-propanol (9:1 v/v), the compound is eluted with a solution of water+2-propanol (1:9, v/v).

After dilution with water and removal of the solvent by lyophilization, compound is obtained as a sodium salt.

### 2. Treatment Method B

The products were dissolved in a THF-water mixture (1:1 vol./vol.). The treatment was run by preparative reverse phase HPLC under the following conditions:

### Column: VYDAC C18, 22×250 mm, 10 μm, 300 Å

### Mobile Phase:

A: Acetonitrile-water (1:1, vol./vol.), 5 mM Tetrabutylammonium phosphate monobasic
B: 2-propanol-water (9:1, vol./vol.), 5 mM Tetrabutylammonium phosphate monobasic
Flow rate: 20 ml/min.

### Elution:

| Time (min) | % mobile phase (B) |
|---|---|
| 0 | 10 |
| 50 | 100 |
| 55 | 10 |
| 60 | 10 |

Detection: UV, 210 nm (wavelength)

Fractions containing the compounds in the form of a tetrabutylammonium salt were collected and concentrated by adsorption on a HPLC, VYDAC C18, 22×250 mm, 10 μm, 300 Å. The sodium salt of the compound is obtained through washing with a 100 mM sodium phosphate dibasic-sodium phosphate monobasic solution in water, pH 7.5+2-propanol (9:1, v/v) (5 volumes)+2-propanol (9:1, v/v) (5 volumes). After removal of the excess of sodium phosphate monobasic-sodium phosphate dibasic by running through 5 volumes of water+2-propanol (9:1 v/v), the compound is eluted with a solution of water+2-propanol (1:9, v/v).

After dilution with water and removal of the solvent by lyophilization, compound is obtained as a sodium salt.

3. Treatment method C

The products were dissolved in a THF-water mixture (1:1 vol./vol.). The treatment was run by preparative reverse phase HPLC under the following conditions:

### Column: VYDAC C18, 22×250 mm, 10 μm, 300 Å

### Mobile Phase:

A: Acetonitrile-water (1:1, vol./vol.), 5 mM Tetrabutylammonium phosphate monobasic
B: 2-propanol-water (9:1, vol./vol.), 5 mM Tetrabutylammonium phosphate monobasic
Flow rate: 20 ml/min.

### Elution:

| Time (min) | % mobile phase (B) |
|---|---|
| 0 | 10 |
| 50 | 100 |
| 55 | 10 |
| 60 | 10 |

Detection: UV, 210 nm (wavelength)

Fractions containing the compounds in the form of a tetrabutylammonium salt were collected and concentrated by adsorption on a HPLC, VYDAC C18, 22×250 mm, 10 μm, 300 Å. The sodium salt of the compound is obtained through washing with a 200 mM sodium phosphate dibasic solution in water, pH 9.2+2-propanol (9:1, v/v) (5 volumes). After removal of the excess of sodium phosphate dibasic by running through 5 volumes of water+2-propanol (9:1 v/v), the compound is eluted with a solution of water+2-propanol (1:9, v/v). After dilution with water and removal of the solvent by lyophilization, compound is obtained as a sodium salt.

### 4. Treatment Method D

The products were dissolved in a THF-water mixture (1:1 vol./vol.). The treatment was run by preparative reverse phase HPLC under the following conditions:

### Column: VYDAC C18, 22×250 mm, 10 μm, 300 Å

### Mobile Phase:

A: Acetonitrile-water (1:1, vol./vol.), 5 mM Tetrabutylammonium phosphate monobasic
B: 2-propanol-water (9:1, vol./vol.), 5 mM Tetrabutylammonium phosphate monobasic
Flow rate: 20 ml/min.

### Elution:

| Time (min) | % mobile phase (B) |
|---|---|
| 0 | 10 |
| 50 | 100 |
| 55 | 10 |
| 60 | 10 |

Detection: UV, 210 nm (wavelength)

Fractions containing the compounds in the form of a tetrabutylammonium salt were collected and concentrated by adsorption on a HPLC, VYDAC C18, 22×250 mm, 10 μm, 300 Å. The sodium salt of the compound is obtained through washing with a 10 g/L sodium chloride solution in water, pH 7.0+2-propanol (9:1, v/v) (5 volumes). After removal of the excess of sodium chloride by running through 5 volumes of water+2-propanol (9:1 v/v), the compound is eluted with a solution of water+2-propanol (1:9, v/v).

After dilution with water and removal of the solvent by lyophilization, compound is obtained as a sodium salt.

### 5. Monitoring of Treatment

After each treatment step, the fractions are analyzed by reverse phase analytic HPLC chromatography according to the following conditions:

### Column: Supelcosil C18, 3 μm, 4.6×150 mm, 100 Å, Supelco

### Mobile Phase:

A: water:acetonitrile (1:1, v/v), 5 mM Tetrabutylammonium phosphate monobasic
B: water-isopropanol (1:9, v/v) 5 mM Tetrabutylammonium phosphate monobasic
Flow rate: 1 ml/min.
Elution: A:B gradient (75:25 to 0:100) within 20 minutes.
Detection: UV, 210 and 254 nm (wavelength)

### Example 1

### IL-6 and TNF-α Secretion by Human Peripheral Blood Mononuclear Cells (PBMC) Effect on IL-6 and TNF-α by Synthetic Compounds of the Invention and Comparison with the Parent Biological Molecule

### Compounds Tested for IL-6 Secretion:

The compounds of the invention tested here for IL-6 secretion were:

Compound 1b (OM-174-DP), compound 16 (OM-174-MP); compound 17 (OM-174-MP-PR), compound 19 (OM-174-MP-PD), and compound 26 (OM-174 MP-AC).

Moreover, the activity of the biological parent molecule, OM-174-DP (P3) was also tested for comparison.

Then, in another series of experiments, the following compounds were tested for TNF-α secretion by human PBMC:

Compound 1b (OM-174-DP), compound 16 (OM-174-MP); compound 17 (OM-174-MP-PR), compound 19 (OM-174-MP-PD), compound 26 (OM-174 MP-AC), compound 41c (OM-174 MP-TE), compound 32 (OM-174 MP-EO), compound 33 (OM-174 MP-EP), and compound 35c (OM-174 MP-CM).

### Introduction and Rational:

The production of IL-6 by human peripheral blood mononuclear cells (PBMC) is an important in vitro test to screen the ability of new compounds to stimulate the immune system. IL-6 is a multifunctional cytokine that plays important roles in host defense, acute phase reactions, and hematopoiesis.

Tumor necrosis factor-(TNF-α) is a pleiotropic cytokine produced by a wide variety of cell types of mostly hematopoietic, but also of non-hematopoietic, origin. TNF-α is necessary for the elimination of numerous infectious agents.

Therefore activation of these cytokines by the compounds of the invention may be of important therapeutic value.

### Methods:

### Preparation of Human PBMC and Cell Culture:

Peripheral blood from healthy donors (Centre de transfusion, Hôpital Universitaire, Geneva) was centrifuged to get the buffy coat. The buffy coat was mixed with Hanks' balanced saline solution (HBSS, Sigma, Buchs, Switzerland), layered over Ficoll Paque Plus (Amersham Pharmacia) to 1.077 g/mL and centrifuged (2800 rpm, 20° C., 25 min). Cells harvested from the interphase were washed twice in HBSS at 800 rpm for 15 min at room temperature and the pelleted cells were resuspended in HBSS. The cell counts were performed using a Neubauer cell. All cell cultures were performed in RPMI-1640 medium supplemented with penicillin (100 U/mL), streptomycin (100 μg/mL), L-glutamine (2 mmol/L) and 10% fetal calf serum (FCS), all obtained from Sigma. For in vitro stimulation, the cells were cultured at a concentration of 1×10⁶ viable cells/well.

### Stimulation and Measurement of IL-6 and TNF-α in Culture Supernatants:

PBMC were incubated at 37° C. and under 5% CO₂ atmosphere with the controls (see below) and with the products of the invention at 1, 5, and 20 μg/mL for IL-6, and at 0.2, 2, and 20 μg/mL for TNF-α secretion. Medium: RPMI alone.

The surpernatants of the cultures were harvested after 24 h and the concentration of IL-6 and TNF-α were measured by an enzyme-linked immunosorbent assay (ELISA) (Human IL-6 and TNF-α ELISA Set, BD OptEIA, San Diego, USA), according to the manufacturer instructions. The detection limits were 10 and 8 pg/mL respectively.

### Results:

The results are shown in the tables below:

### A: For IL-6

TABLE 1.1
Negative (medium) and Positive (LPS) controls on IL-6
production ± STDEV (in pg/ml) by human peripheral
blood mononuclear cells.

| Products (number) + [batch] + | Conc. | | |
|---|---|---|---|
| concentration μg/ml | μg/ml | IL6 (pg/ml) | stdev |
| Medium | NA | 6.5 | 0.2 |
| Medium | NA | 20.7 | 1.5 |
| LPS [026:B6] 0.001 μg/ml | 0.001 | 15105 | 627.7 |
| LPS [026:B6] 0.01 μg/ml | 0.01 | 13194 | 632.9 |
| LPS [026:B6] 0.1 μg/ml | 0.1 | 16097 | 1004.7 |

### Interpretation of the Results:

LPS induced as expected very high levels of IL-6 from human PBMC, even at the lowest dose tested.

TABLE 1.2
Effect of the synthetic compound (1b) of the invention (OM-174-DP)
on IL-6 production by human PBMC, in comparison to the biological
parent molecule (174-P3).

| Products (number) + [batch] + | Conc. | | |
|---|---|---|---|
| concentration μg/ml | μg/ml | IL6 (pg/ml) | stdev |
| OM-174-DP [P3] 1 μg/ml | 1 | 9.8 | 1.9 |
| OM-174-DP [P3] 5 μg/ml | 5 | 18.5 | 4.2 |
| OM-174-DP [P3] 20 μg/ml | 20 | 97.7 | 12.6 |
| (1b) OM-174-DP [SMOR-189] 1 μg/ml | 1 | 20.2 | 2.0 |
| (1b) OM-174-DP [SMOR-189] 5 μg/ml | 5 | 57.1 | 2.8 |
| (1b) OM-174-DP [SMOR-189] 20 μg/ml | 20 | 225.5 | 7.9 |

### Interpretation of the Results:

Both batches of OM-174 induce the secretion of IL-6 from human PBMC, and the levels obtained with the synthetic molecule were somewhat higher than those obtained with the biological molecule P3.

TABLE 1.3
Effect of 5 examples of synthetic compounds of the invention
on IL-6 production by human PBMC

| Products (number) + [code] + | Conc. | IL6 | |
|---|---|---|---|
| concentration μg/ml | μg/ml | (pg/ml) | stdev |
| (1b) OM-174-DP [SMOR-189] 1 μg/ml | 1 | 20.2 | 2.0 |
| (1b) OM-174-DP [SMOR-189] 5 μg/ml | 5 | 57.1 | 2.8 |
| (1b) OM-174-DP [SMOR-189] 20 μg/ml | 20 | 225.5 | 7.9 |
| (16) OM-174-MP [SMORII-30] 1 μg/ml | 1 | 81.1 | 4.5 |
| (16) OM-174-MP [SMORII-30] 5 μg/ml | 5 | 473.3 | 32.0 |
| (16) OM-174-MP [SMORII-30] 20 μg/ml | 20 | 1348.2 | 22.8 |
| (17) OM-174-MP-PR [SMORII-24] 1 μg/ml | 1 | 171.6 | 9.1 |
| (17) OM-174-MP-PR [SMORII-24] 5 μg/ml | 5 | 447.6 | 45.5 |
| (17) OM-174-MP-PR [SMORII-24] 20 μg/ml | 20 | 752.4 | 37.2 |
| (19) OM-174-MP-PD [SMORII-32] 1 μg/ml | 1 | 44.5 | 1.3 |
| (19) OM-174-MP-PD [SMORII-32] 5 μg/ml | 5 | 95.9 | 1.9 |
| (19) OM-174-MP-PD [SMORII-32] 20 μg/ml | 20 | 199.1 | 6.4 |
| (26) OM-174-MP-AC-[F4] 1 μg/ml | 1 | 269.0 | 10.2 |
| (26) OM-174-MP-AC [F4] 5 μg/ml | 5 | 1923.2 | 69.3 |
| (26) OM-174-MP-AC-[F4] 20 μg/ml | 20 | 8349.3 | 483.5 |

### Interpretation of the Results:

In comparison to the biological parent molecule OM-174-DP (batch P3), the synthesized molecule (1b) induces higher levels of IL-6 by human monocytes.

The same is true for OM-174-MP (16), since the corresponding biological product induced no production of IL-6, even at the highest dose tested (20 μg/ml, not shown), whereas the related synthetic molecule OM-174-MP (compound 16) induces up to 1348 pg/ml of IL-6.

In general, all the synthetic molecules tested (1b, 16, 17, 19, and 26) were able to induce IL-6 secretion.

### B) For TNF-α

TABLE 1.4
Negative (medium) and Positive (LPS) controls on TNF-α
production ± STDEV (in pg/ml) by human peripheral blood
mononuclear cells.

| Products (number) + code + | Conc. | | |
|---|---|---|---|
| concentration μg/ml | μg/ml | TNF-α (pg/ml) | stdev |
| Medium | NA | 24 | 6.8 |
| Medium | NA | 29 | 8.8 |
| LPS [026:B6] 0.2 μg/ml | 0.2 | 16112 | 411 |
| LPS [026:B6] 2 μg/ml | 2 | 14237 | 494 |
| LPS [026:B6] 20 μg/ml | 20 | 13602 | 472 |

### Interpretation of the Results:

LPS induced as expected very high levels of TNF-α from human PBMC, at the three doses tested.

TABLE 1.5
Effect of the synthetic compound (1b) of the invention (OM-174-DP)
on TNF-α production by human PBMC, in comparison to the
biological parent molecule (174-P3).

| Products (number) + code + | Conc. | TNF-α | |
|---|---|---|---|
| concentration μg/ml | μg/ml | (pg/ml) | stdev |
| OM-174-DP [P3] 0.2 μg/ml | 0.2 | 2.5 | 2.1 |
| OM-174-DP [P3] 2 μg/ml | 2 | 7.1 | 1.1 |
| OM-174-DP [P3] 20 μg/ml | 20 | 300 | 31 |
| (1b) OM-174-DP [SMORII-132] 0.2 μg/ml | 0.2 | 37 | 4.9 |
| (1b) OM-174-DP [SMORII-132] 2 μg/ml | 2 | 117 | 22 |
| (1b) OM-174-DP [SMORII-132] 20 μg/ml | 20 | 1105 | 69 |

### Interpretation of the Results:

Both batches of OM-174 induce the secretion of TNF-α from human PBMC, and the levels obtained with the synthetic molecule were somewhat higher than those obtained with the biological molecule P3.

TABLE 1.6
Effect of 9 examples of synthetic compounds of the invention
on TNF-α production by human PBMC

| Products (number) + code + | Conc. | TNF-α | |
|---|---|---|---|
| concentration μg/ml | μg/ml | (pg/ml) | stdev |
| (1b) OM-174-DP [SMORII-132] 0.2 μg/ml | 0.2 | 37 | 4.9 |
| (1b) OM-174-DP [SMORII-132] 2 μg/ml | 2 | 117 | 22 |
| (1b) OM-174-DP [SMORII-132] 20 μg/ml | 20 | 1105 | 69 |
| (16) OM-174-MP [SMORII-30] 0.2 μg/ml | 0.2 | 0.69 | 0.1 |
| (16) OM-174-MP [SMORII-30] 2 μg/ml | 2 | 49 | 14 |
| (16) OM-174-MP [SMORII-30] 20 μg/ml | 20 | 1705 | 16 |
| (17) OM-174-MP-PR [KAS1-108] 0.2 μg/ml | 0.2 | 46 | 4.6 |
| (17) OM-174-MP-PR [KAS1-108] 2 μg/ml | 2 | 474 | 16 |
| (17) OM-174-MP-PR [KAS1-108] 20 μg/ml | 20 | 2114 | 35 |
| (19) OM-174-MP-PD [SMORII-113] 0.2 μg/ml | 0.2 | 9.2 | 4.1 |
| (19) OM-174-MP-PD [SMORII-113] 2 μg/ml | 2 | 5.1 | 2.7 |
| (19) OM-174-MP-PD [SMORII-113] 20 μg/ml | 20 | 11.2 | 6.0 |
| (26) OM-174-MP-AC [KAS1-103] 0.2 μg/ml | 0.2 | 29.3 | 3.6 |
| (26) OM-174-MP-AC [KAS1-103] 2 μg/ml | 2 | 1264 | 33 |
| (26) OM-174-MP-AC [KAS1-103] 20 μg/ml | 20 | 7016 | 186 |
| (41c) OM-174-MP-TE [KAS2-10] 0.2 μg/ml | 0.2 | 570 | 34 |
| (41c) OM-174-MP-TE [KAS2-10] 2 μg/ml | 2 | 6036 | 306 |
| (41c) OM-174-MP-TE [KAS2-10] 20 μg/ml | 20 | 9769 | 119 |
| (32) OM-174-MP-EO [SMORII-74] 0.2 μg/ml | 0.2 | 5.5 | 3.5 |
| (32) OM-174-MP-EO [SMORII-74] 2 μg/ml | 2 | 7.8 | 6.4 |
| (32) OM-174-MP-EO [SMORII-74] 20 μg/ml | 20 | 251 | 20 |
| (33) OM-174-MP-EP [SMORII-83] 0.2 μg/ml | 0.2 | 8.0 | 1.4 |
| (33) OM-174-MP-EP [SMORII-83] 2 μg/ml | 2 | 8.3 | 1.7 |
| (33) OM-174-MP-EP [SMORII-83] 20 μg/ml | 20 | 21 | 4.7 |
| (35c) OM-174-MP-CM [SMORII-135] 0.2 μg/ml | 0.2 | 48 | 3.6 |
| (35c) OM-174-MP-CM [SMORII-135] 2 μg/ml | 2 | 502 | 11 |
| (35c) OM-174-MP-CM [SMORII-135] 20 μg/ml | 20 | 591 | 11 |

### Interpretation of the Results:

With the exception of the synthesized molecules (19) and (33), all the synthesized molecules (1b, 16, 17, 26, 41c, and 32) induced high levels of TNF-α by human monocytes, suggesting that they could act as immunostimulating drugs.

The results suggest also that molecules (19, e.i. OM-174-MP-PD) and (33, e.i. OM-174-MP-EP) could be worth to be developed as “anti-inflammatory” drugs. Very interestingly, both compounds (19) and (33) exhibited “anti-inflammatory” properties.

Indeed, compound (19) displayed anti-asthmatic properties both “prophylactically” and “therapeutically” in a model of LACK-induced asthma (see example 6), and inhibited the release of compound 48/80-induced histamine secretion by murine mast cells (see example 7). In this later model presented in example 7, compound (33) was also active.

### Example 2

### Modification of the Biological Activity of the Biological Compound OM-174-DP

### Enhancement of TNF-α Induced Secretion by THP-1 Cells by an Original Purification Method of the Parent Biological Molecule OM-174-DP

### Compounds Tested:

The compounds of the invention tested here are:

The parent biological batch (GMP004) of the molecule of the invention OM-174-DP was tested either from the stock solution, or re-purified as described below, mainly by varying the pH of the HPLC mobile phase.

### Introduction and Rational:

Tumor necrosis factor-(TNF-α) is a pleiotropic cytokine produced by a wide variety of cell types of mostly hematopoietic, but also of non-hematopoietic, origin. TNF-α is necessary for the elimination of numerous infectious agents (Candida albicans, Listeria monocytogenes, mycobacteria . . . ), and exerts potent proinflammatory effects, e.g. by inducing the expression of adhesion molecules such as VCAM-1, intercellular adhesion molecule 1 (ICAM-1), or E-selectin on endothelial cells and other cell types.

Overproduction of TNF, however, has been also implicated in the pathogenesis of various diseases, such as rheumatoid arthritis, insulin-dependent diabetes-mellitus, and inflammatory bowel disease, in particular Crohn's disease.

Therefore secretion of TNF-α is necessary to trigger immunological responses; however this production should be mastered in order to avoid inflammatory pathologies.

One batch of the original biological product OM-174-DP (GMP004) was reformulated according to the two methods described below:

### Methods

### 1. Method A

The purification was run by preparative reverse phase HPLC. The UV detection was done at 210 nm. Fractions containing the compounds in the form of a tetrabutylammonium salt were collected and concentrated by adsorption on a HPLC. The sodium salt of the compound is obtained through washing with a 200 mM sodium phosphate monobasic solution in water, pH 4.23+2-propanol (9:1, v/v) (5 volumes). After removal of the excess of sodium phosphate monobasic by running through 5 volumes of water+2-propanol (9:1 v/v), the compound is eluted with a solution of water+2-propanol (1:9, v/v). After dilution with water and removal of the solvent by lyophilization, compound is obtained as a sodium salt.

The compounds obtained were then tested, with or without pH adjustment (at 7.5) on THP-1 cells to analyze their potential to induce TNF-α secretion (see below).

### 2. Method B

The purification was run by preparative reverse phase HPLC. The UV detection was done at 210 nm. Fractions containing the compounds in the form of a tetrabutylammonium salt were collected and concentrated by adsorption on a HPLC. The sodium salt of the compound is obtained through washing with a 100 mM sodium phosphate dibasic-sodium phosphate monobasic solution in water, pH 7.5+2-propanol (9:1, v/v) (5 volumes)+2-propanol (9:1, v/v) (5 volumes). After removal of the excess of sodium phosphate monobasic-sodium phosphate dibasic by running through 5 volumes of water+2-propanol (9:1 v/v), the compound is eluted with a solution of water+2-propanol (1:9, v/v).

After dilution with water and removal of the solvent by lyophilization, compound is obtained as a sodium salt.

The compounds obtained were then tested, with or without pH adjustment (at 7.5) on THP-1 cells to analyze their potential to induce TNF-α secretion (see below).

### THP-1 Cell Culture:

THP-1, a human leukemic monocytic cell line, was obtained from ATCC (Manassas, USA)

THP-1 cells (10⁶ cells/ml, 200 ll/well) were cultured in 96-well flat-bottomed tissue culture plate (Costar) in RPMI medium supplemented with 10% human serum (HS; Gibco-BRL), containing 10 mM HEPES buffer, 1 mM pyruvate, 0.1 M nonessential amino acids, 2 mM glutamine, 50 mM of 2-mercaptoethanol, 100 U/ml penicillin, and 10 mg/ml streptomycin (complete medium). Cells were stimulated with different concentrations of the compounds of the invention for various times at 37° C. in a humidified incubator with 5% CO₂. Culture supernatants were harvested and stored at −20° C. until cytokines determination by ELISA.

### Stimulation and Measurement of TNF-α in Culture Supernatants:

Cells were incubated at 37° C. and under 5% CO₂ atmosphere with the products of the invention at 0.2, 2, and 20 μg/mL: medium: RPMI alone.

The surpernatants of the cultures were harvested after 24 h and the concentration of TNF-α was measured by an enzyme-linked immunosorbent assay (ELISA) (BD OptEIA, San Diego, USA), according to the manufacturer instructions. The detection limit was 8 pg/mL.

### Results:

The results are shown in Table 2.1 below:

TABLE I
Effect of the compounds of the invention on
TNF-α production by THP-1 cells

| | TNF α | |
|---|---|---|
| Product [batch]/method/dose/pH | (pg/ml) | stdev |
| Medium | 0.51 | 0.33 |
| Medium | 0.68 | 1.24 |
| OM-174-DP [GMP004] (0.2 μg/ml) | 13.03 | 0.39 |
| OM-174-DP [GMP004] (2 μg/ml) | 64.42 | 4.81 |
| OM-174-DP [GMP004] (20 μg/ml) | 193.02 | 14.68 |
| OM-174-DP [GMP004] by method A (0.2 μg/ml) | 10.85 | 3.59 |
| OM-174-DP [GMP004] by method A (2 μg/ml) | 11.20 | 12.56 |
| OM-174-DP [GMP004] by method A (20 μg/ml) | 74.90 | 7.22 |
| OM-174-DP [GMP004] by method A (0.2 μg/ml), | 1.22 | 0.46 |
| then pH 7.5 | | |
| OM-174-DP [GMP004] by method A (2 μg/ml), | 9.09 | 1.20 |
| then pH 7.5 | | |
| OM-174-DP [GMP004] by method A (20 μg/ml), | 96.10 | 4.80 |
| then pH 7.5 | | |
| OM-174-DP [GMP004] by method B (0.2 μg/ml) | 205.96 | 43.94 |
| OM-174-DP [GMP004] by method B (2 μg/ml) | 447.36 | 6.00 |
| OM-174-DP [GMP004] by method B (20 μg/ml) | 591.09 | 23.14 |
| OM-174-DP [GMP004] by method B (0.2 μg/ml), | 141.55 | 3.86 |
| then pH 7.5 | | |
| OM-174-DP [GMP004] by method B (2 μg/ml), | 473.89 | 18.56 |
| then pH 7.5 | | |
| OM-174-DP [GMP004] by method B (20 μg/ml), | 636.78 | 51.78 |
| then pH 7.5 | | |

### Interpretation of the Results:

The TNF-α value obtained with the OM-174-DP biological products [GMP004] at 20 μg/ml was 193 pg/ml. Here we demonstrate the purification method B enhances the biological activity of the parent biological product by a factor of 3.

In conclusion, the method of purification described here could therefore be adapted to the clinics to enhance the therapeutic activity of the drug.

### Example 3

### Effect of the Biological Activity of 3 Synthetic Monophosphoryl Compounds of the Invention

### Nitric Oxide Production by Murine Macrophages Stimulated by 3 Monophosphoryl Synthetic Compounds of the Invention

### Compounds Tested:

The compounds of the invention presented here are:

Compound 16 (OM-174-MP); compound 17 (OM-174-MP-PR), and compound 19 (OM-174-MP-PD).

### Introduction and Rational:

The production of the Nitric oxide (NO) by macrophages is an important in vitro test to screen the ability of new compounds to stimulate the immune system. It is an important signaling molecule in the body of mammals including humans, one of the few gaseous signaling molecules known.

The nitric oxide molecule is a free radical, which makes it very reactive and unstable. In the body, nitric oxide is synthesized from arginine and oxygen by various nitric oxide synthase (NOS) enzymes and by sequential reduction of inorganic nitrate.

Macrophages produce nitric oxide in order to kill invading bacteria. Under certain conditions, this can backfire: Fulminant infection (sepsis) causes excess production of nitric oxide by macrophages, leading to vasodilatation (widening of blood vessels), probably one of the main causes of hypotension (low blood pressure) in sepsis.

The biological functions of nitric oxide were discovered in the 1980s, and nitric oxide was named “Molecule of the Year” in 1992 by the journal Science. It is estimated that yearly about 3,000 scientific articles about the biological roles of nitric oxide are published.

Therefore its activation by the compounds of the invention may be of important therapeutic value.

### Material and Methods:

Experimental assay of nitric oxide production by murine macrophages: Six-week old male C57/BL6 mice (six weeks old male, SPF quality, Charles Rivier, FR) were killed by CO2 inhalation. The hip, femur, and tibia from the posterior appendage were removed. The bone marrow was extracted from the lumen by injecting Dulbecco's Modified Eagle Medium (DH) through the bone after cutting both end portions. After washing, the stem cells were resuspended (40,000 cells/mL) in DH medium supplemented with 20% horse serum and 30% L929 cell supernatant. The cell suspension is incubated for 8 days in an incubator at 37° C. under 8% CO2 and moisture-saturated atmosphere. Macrophages are then detached with ice-cold PBS, washed and resuspended in DH medium supplemented with 5% fetal calf serum (FCS), amino acids and antibiotics (DHE medium). The cell density is adjusted to 700,000 cells/mL. Aqueous solutions of the products are serially diluted in DHE medium directly in microtiter plates. The products are tested in triplicates and each microtiter plate comprises a negative control composed of medium. The final volume in each well is 100 μL. 100 μL of the cell suspension are added to the diluted products and the cells are incubated for 22 h in an incubator at 37° C., under 8% CO2 and a moisture-saturated atmosphere. At the end of the incubation period, 100 μL of supernatant are transferred to another microtiter plate and the nitrite concentration produced in each supernatant is determined by running a Griess reaction. 100 μL of Griess reagent (5 mg/mL of sulfanilamide+0.5 mg/mL of N-(1-naphtyl)ethylene-diamine hydrochloride) in 2.5% aqueous phosphoric acid, are added to each well. The microtiter plates are read with a spectrophotometer (SpectraMax Plus, Molecular Devices) at 562 nm against a reference at 690 nm. The nitrite concentration is proportional to nitric oxide content being formed. The nitrite content is determined based on a standard curve. The results are given as mean value±standard deviation and plotted as a dose response curve.

### Results:

The results are shown in FIG. 25. The three molecules tested were able to induce high levels of nitric oxide by murine macrophages. Compound 19 (OM-174-MP-PD) was active at lower doses (from 0.01 μg/ml) in this test than compounds 16 (OM-174-MP) and 17 (OM-174-MP-PR).

### Example 4

### Effect of the Purification According to Method B on IL-6 Production by Human Peripheral Blood Mononuclear Cells of a Previously Inactive Synthetic Compounds of the Invention, Obtained Via Method D

### Compounds Tested:

The compounds of the invention presented here are:

Batch 14 of the synthetic product OM-174-DP (compound 1b), re-processed or not re-processed according to method D (see below).

### Introduction and Rational:

The batch of the compound presented here (synthetic OM-174-DP) was originally inactive because it was obtained with method D, in which the final pH is not well mastered. Indeed batch 14 had a pH of 4.88, before to undergo purification according to method B (see below). Very interestingly, the method of purification B (see example 2) increased considerable the activity of batch 14.

See also Example 1 for a description on IL-6 biological effects.

### Method of Purification D

The method was described in detail above.

The synthetic product OM-174-DP (1b) products (batch 14) was dissolved in a THF-water mixture (1:1 vol./vol.). The purification was run by preparative reverse phase HPLC and the UV detection was performed at 210 nm.

Fractions containing the compounds in the form of a tetrabutylammonium salt were collected and concentrated by adsorption on a HPLC, VYDAC C18, 22×250 mm, 10 μm, 300 Å. The sodium salt of the compound is obtained through washing with a 10 g/L sodium chloride solution in water, pH 7.0+2-propanol (9:1, v/v) (5 volumes). After removal of the excess of sodium chloride by running through 5 volumes of water+2-propanol (9:1 v/v), the compound is eluted with a solution of water+2-propanol (1:9, v/v).

After dilution with water and removal of the solvent by lyophilization, compound is obtained as a sodium salt. By using this method (D), the final pH is not well controlled. Indeed, the pH of batch 14 was 4.88.

Then this batch was retreated according to method B (see below).

### Method of Purification B

The method was described in detail above. The purification was run by preparative reverse phase HPLC. The UV detection was done at 210 nm. Fractions containing the compounds in the form of a tetrabutylammonium salt were collected and concentrated by adsorption on a HPLC. The sodium salt of the compound is obtained through washing with a 100 mM sodium phosphate dibasic-sodium phosphate monobasic solution in water, pH 7.5+2-propanol (9:1, v/v) (5 volumes)+2-propanol (9:1, v/v) (5 volumes). After removal of the excess of sodium phosphate monobasic-sodium phosphate dibasic by running through 5 volumes of water+2-propanol (9:1 v/v), the compound is eluted with a solution of water+2-propanol (1:9, v/v).

After dilution with water and removal of the solvent by lyophilization, compound is obtained as a sodium salt.

The compounds obtained were then tested, with or without pH adjustment (at 7.5) on THP-1 cells to analyze their potential to induce TNF-α secretion (see below).

### Preparation of Human PBMC and Cell Culture:

Peripheral blood from healthy donors (Centre de transfusion, Hôpital Universitaire, Geneva) was centrifuged to get the buffy coat. The buffy coat was mixed with Hanks' balanced saline solution (HBSS, Sigma, Buchs, Switzerland), layered over Ficoll Paque Plus (Amersham Pharmacia) to 1.077 g/mL and centrifuged (2800 rpm, 20° C., 25 min). Cells harvested from the interphase were washed twice in HBSS at 800 rpm for 15 min at room temperature and the pelleted cells were resuspended in HBSS. The cell counts were performed using a Neubauer cell. All cell cultures were performed in RPMI-1640 medium supplemented with penicillin (100 U/mL), streptomycin (100 μg/mL), L-glutamine (2 mmol/L) and 10% fetal calf serum (FCS), all obtained from Sigma. For in vitro stimulation, the cells were cultured at a concentration of 1×10⁶ viable cells/well.

### Stimulation and Measurement of IL-6 in Culture Supernatants:

PBMC are incubated at 37° C. and under 5% CO2 atmosphere with the products of the invention.

The supernatants of the cultures are harvested after 24 h and the concentration of IL-6 was measured by an enzyme-linked immunosorbent assay (ELISA) (Human IL-6 ELISA Set, BD OptEIA, San Diego, USA), according to the manufacturer instructions. The detection limit was 10 pg/mL.

### Results:

The results are shown in the FIG. 26 which shows the application of method B (i.e. the use of an appropriate pH during the purification procedure) to the compound of the invention (here compound 1b) transforms the inactive compound (batch 14) into a fully efficient activator of human PBMC (batch 39).

### Example 5

### Modification of the Biological Activity of compound 1b (OM-174-DP)

### Enhancement of TNF-α Induced Secretion by THP-1 Cells Differentiated into Macrophages by an Original Purification Method of Various Batches of the Molecule OM-174-DP

### Compounds Tested:

The compounds of the invention presented here are:

Two biological batches (P3 and GMP004) of compound 1b (OM-174-DP), and the following synthetic batch (14) was reprocessed according to methods A or B (see below). LPS was used as positive control.

### Introduction and Rational:

### TNF-α

Tumor necrosis factor-(TNF-α) is a pleiotropic cytokine produced by a wide variety of cell types of mostly hematopoietic, but also of nonhematopoietic, origin. TNF-α is necessary for the elimination of numerous infectious agents (Candida albicans, Listeria monocytogenes, mycobacteria . . . ), and exerts potent proinflammatory effects, e.g. by inducing the expression of adhesion molecules such as VCAM-1, intercellular adhesion molecule 1 (ICAM-1), or E-selectin on endothelial cells and other cell types.

Overproduction of TNF, however, has been also implicated in the pathogenesis of various diseases, such as rheumatoid arthritis, insulin-dependent diabetes-mellitus, and inflammatory bowel disease, in particular Crohn's disease.

Therefore secretion of TNF-α is necessary to trigger immunological responses, however this production should be mastered in order to avoid inflammatory pathologies. The inactive synthetic batch (batch “14”, see example 4) was reformulated according to the two methods described below:)

### Methods

### 1. Method A

The purification was run by preparative reverse phase HPLC. The UV detection was done at 210 nm. Fractions containing the compounds in the form of a tetrabutylammonium salt were collected and concentrated by adsorption on a HPLC. The sodium salt of the compound is obtained through washing with a 200 mM sodium phosphate monobasic solution in water, pH 4.23+2-propanol (9:1, v/v) (5 volumes). After removal of the excess of sodium phosphate monobasic by running through 5 volumes of water+2-propanol (9:1 v/v), the compound is eluted with a solution of water+2-propanol (1:9, v/v).

After dilution with water and removal of the solvent by lyophilization, compound is obtained as a sodium salt.

The compounds obtained were then tested, with or without pH adjustment (at 7.5) on THP-1 cells to analyse their potential to induce TNF-α secretion (see below).

### 2. Method B

The purification was run by preparative reverse phase HPLC. The UV detection was done at 210 nm. Fractions containing the compounds in the form of a tetrabutylammonium salt were collected and concentrated by adsorption on a HPLC. The sodium salt of the compound is obtained through washing with a 100 mM sodium phosphate dibasic-sodium phosphate monobasic solution in water, pH 7.5+2-propanol (9:1, v/v) (5 volumes)+2-propanol (9:1, v/v) (5 volumes). After removal of the excess of sodium phosphate monobasic-sodium phosphate dibasic by running through 5 volumes of water+2-propanol (9:1 v/v), the compound is eluted with a solution of water+2-propanol (1:9, v/v).

After dilution with water and removal of the solvent by lyophilization, compound is obtained as a sodium salt.

The compounds obtained were then tested, with or without pH adjustment (at 7.5) on THP-1 cells to analyse their potential to induce TNF-α secretion (see below).

The compounds obtained were then tested, with or without pH adjustment (at 7.5) on THP-1 cells to analyse their potential to induce TNF-α secretion (see below).

### THP-1 Cell Culture:

THP-1 cells (see method in example 1) are culture (5×10⁵ cellules/ml) in RPMI with 10% FCS+100 ng/ml PMA (Sigma). After 3 days adherents cells were harvested and adjusted at the concentration of 3×10⁵ cells per well and incubated with the products at 37° C. with 5% CO2 during 6 hours.

The surpernatants of the cultures were harvested after 24 h and the concentration of TNF-α was measured by an enzyme-linked immunosorbent assay (ELISA) (BD OptEIA, San Diego, USA), according to the manufacturer instructions. The detection limit was 8 pg/mL.

### Results:

The results are separated below in 3 different tables:

TABLE 5.1
TNF-alpha production by THP-1 cells differentiated into
macrophages by medium, LPS, and the biological
batches GMP004 et P3 of the parent product OM-174-DP

| Product [batch] dose μg/ml | μg/ml | mean | | stdev |
|---|---|---|---|---|
| Medium | 0 | 3637 | ± | 145 |
| LPS 0.001 | 0.001 | 5400 | ± | 1989 |
| LPS 0.01 | 0.01 | 8770 | ± | 687 |
| LPS 0.1 | 0.1 | 53165 | ± | 2536 |
| OM-174-DP [GMP004] 2 | 2 | 5045 | ± | 2275 |
| OM-174-DP [GMP004] 20 | 20 | 10150 | ± | 3044 |
| OM-174-DP [P3] 2 | 2 | 4628 | ± | 206 |
| OM-174-DP [P3] 20 | 20 | 9579 | ± | 2381 |

### Interpretation of the Results:

LPS induces, as expected, high levels of TNF-alpha. The production of TNF-alpha induced by the two biological batches (P3 and GMP 004) of OM-174 were much lower.

TABLE 5.2
Comparison of the TNF-alpha production by THP-1 cells
differentiated into macrophages by the biological batch
GMP004, before and after the purification via the
method A or method B of the invention (to give batches 54).

| Product [batch], method, | | | | |
|---|---|---|---|---|
| dose μg/ml | μg/ml | mean | | stdev |
| Medium | 0 | 3637 | ± | 145 |
| OM-174-DP [GMP004] 2 | 2 | 5045 | ± | 2275 |
| OM-174-DP [GMP004] 20 | 20 | 10150 | ± | 3044 |
| OM-174-DP [SMORII-54]A1 2 | 2 | 4208 | ± | 308 |
| OM-174-DP [SMORII-54] A1 20 | 20 | 10223 | ± | 2142 |
| OM-174-DP [SMORII-54] A2 2 | 2 | 4328 | ± | 421 |
| OM-174-DP [SMORII-54] A2 20 | 20 | 22802 | ± | 5612 |
| OM-174-DP [SMORII-54] B1 0.2 | 0.2 | 19039 | ± | 5497 |
| OM-174-DP [SMORII-54] B1 2 | 2 | 22049 | ± | 3442 |
| OM-174-DP [SMORII-54] B1 20 | 20 | 43301 | ± | 3069 |
| OM-174-DP [SMORII-54] B2 0.2 | 0.2 | 8079 | ± | 1704 |
| OM-174-DP [SMORII-54] B2 2 | 2 | 11401 | ± | 694 |
| OM-174-DP [SMORII-54] B2 20 | 20 | 22513 | ± | 2584 |

### Interpretation of the Results:

The results clearly show that the application of the method B to the batch GMP004 strongly increases the activity of the biological parent batch GMP004.

TABLE 5.3
Comparison of the TNF-alpha production by THP-1 cells differentiated
into macrophages by the originally inactive synthetic batch (SMORII 14)
of OM-174-DP (see example 4), and clear enhancement of its activity
by the method B of the invention (generation of the “39” series).

| Product [batch], dose μg/ml | μg/ml | mean | | stdev |
|---|---|---|---|---|
| Medium | 0 | 3637 | ± | 145 |
| OM-174-DP [SMORII-14-150306] 2 | 2 | 14603 | ± | 1030 |
| OM-174-DP [SMORII-14-150306] 20 | 20 | 9582 | ± | 2243 |
| OM-174-DP [SMORII-14-So] 2 | 2 | 8853 | ± | 2328 |
| OM-174-DP [SMORII-14-So] 20 | 20 | 9894 | ± | 3319 |
| OM-174-DP [SMORII-39-060509] 2 | 2 | 23331 | ± | 3019 |
| OM-174-DP [SMORII-39-060509] 20 | 20 | 37637 | ± | 3945 |

### Interpretation of the Results:

The products coded as batch “39” were obtained from the synthetic compound named batch “14” via processes to the method B. Clearly the process B enhanced the activity of the parent molecule. In contrast, a sonication procedure is without effect (series “so).

### Example 6

### Effect of Synthetic OM-174-DP and Synthetic OM-174-MP-PD in a Model of LACK-Induced Asthma, “Prophylactly” and “Therapeutically”

An example in vivo of the biological activity of two representative compounds of the invention is presented here. Using a mouse model of allergic asthma previously published (described in Julia et al. Immunity. 2002 February; 16(2):271-83), we aimed to investigate whether i.p. administrations of the synthetic molecules OM-174-DP and OM-174-MP-PD would inhibit airway inflammation in LACK-sensitized and challenged mice. To this goal, mice were treated either all along asthma induction (prophylactic model) or therapeutically (i.e. three times after animals have been sensitized to the allergen, the protein LACK). As read-outs, eosinophils were enumerated in bronchioalveolar lavages (BAL), and the well known markers of allergic asthma, namely the Th2 cytokines IL-4, IL-5, and IL-13 were quantified from the lungs. Moreover the level of plasmatic IgE was also reported.

### Protocol:

### Material

Saline solution were given in aerosols as control

Recombinant LACK protein was produced in E. coli, and purified onto a Ni-NTA affinity column, as described (Mougneau et al., 1995).

Aluminium hydroxide (Alum) was purchased from Pierce

The cytocentrifuge, is a Cytospin 4 (Thermo-Shandon, Cheschire, U.K.), cytoslides are purchased from Thermo-Shandon and Wright and Giemsa stains from Sigma.

Aerosols were given using an ultra-son nebulizer Ultramed (Medicalia, Forenze, Italy)

Anti-IgE (R35-118) coupled to biotin was purchased from BD Biosciences (Le Pont de Claix, France).

### Animals

6 weeks old female BALB/c ByJ mice were purchased from The Centre d'Elevage Janvier, France. The mice were kept under specific-pathogen free conditions and were fed with a standard diet provided by Safe (Augy, France).

### Experimental Groups

The 5 following groups were tested:

A: NEGATIVE Control:

untreated LACK-sensitized and saline-challenged mice (3 mice)

B: POSITIVE Control:

untreated LACK-sensitized and challenged mice (6 mice)

C: 174-DP Prophylactic:

OM-174-DP (i.p.)-treated LACK-sensitized and challenged mice (6 mice)

### D: 174-MP-PD Prophylactic:

OM-174-MP-PD (i.p.)-treated LACK-sensitized and challenged mice (6 mice)

E: 174-DP Therapeutic:

OM-174-DP (i.p.)-treated LACK-sensitized and challenged mice (6 mice)
Treatment and Schedule with Test or Control Article

Experiment started at day 0. On days 0, 2, 3, 4, 7, 9, 10 11, and 12, mice of groups C, and D were treated i.p. with synthetic OM-174-DP (compound 1b) and OM-174-MP-PD (compound 19) respectively at the dose of 1 mg/Kg (20 μg per mouse).

Mice of groups E were treated therapeutically i.p. on days 15, 17 and 19 at the dose of 1 mg/Kg (20 μg per mouse).

On day 1 and day 8, mice were sensitized i.p. with LACK/Alum. From day 16 to day 20, all the groups except group A mice were challenged with aerosols of a solution of LACK (0.15%). Group A received a saline solution (NaCl 0.9%) (group A) for 40 minutes instead.

### Method

### A: Bronchioalveolar Lavages (BAL) and Eosinophil Counts.

For all the animals, Broncho-alveolar lavages (BAL) were performed. Two days after the last aerosol challenge, mice were bled and a canula was inserted into their trachea. Lungs were washed 3 times with 1 ml of warmed PBS. Cells were washed with PBS, and red blood cells were lysed using a red blood cell lysis buffer. Cells were further washed in PBS and counted. For differential BAL cell counts, cytospin preparations were made and stained with Wright/Giemsa. At least 400 cells were scored for each slide, and the numbers of lymphocytes, neutrophils, eosinophils, and macrophages/DC/pneumocytes (scored as other mononuclear cells) were determined by microscopic examination. Only eosinophilia is reported here.

### B: Pulmonary Cytokines Determination:

To analyze pulmonary cytokine contents, lungs were harvested and left lungs were used to prepare protein extracts. 400 μl were recovered for each left lung. Cytokines (IL-4 and IL-13 in a first series of analyses, and then IL-5 and IFN-γ) were measured by multiplex analysis using FACSArray. The results, normalized for the protein content, are presented in pg/ml.

### C: LACK-Specific IgE Determination:

To analyze specific IgE contents, mice of groups A, B, and G were bled by heart puncture two days after the last aerosol, and sera were prepared. LACK-specific IgE were measured by ELISA.

### Results:

### A) Eosinophilia

### Characterization of Number of Eosinophils in Broncho-Alveolar Lavages:

The results from each individual mouse, and the mean value of each group tested are shown in the table below

| Eosino × 10⁶ | mouse 1 | mouse 2 | mouse 3 | mouse 4 | mouse 5 | mouse 6 | Mean ± SEM |
|---|---|---|---|---|---|---|---|
| A: NEG | 0 | 0 | 0 | ND | ND | ND | 0 |
| CTRL | | | | | | | |
| B: POS | 1445409 | 218957 | 286483 | 664662 | 1831421 | 895714 | 890441 ± 641887* |
| CTRL | | | | | | | |
| C: OM-174- | 75359 | 385612 | 33412 | 54158 | 105352 | 4792 | 109781 ± 139453* |
| DP (1b) | | | | | | | |
| Prophylactic | | | | | | | |
| D: OM-174- | 33286 | 64010 | 47018 | 252294 | 82555 | 123305 | 100411 ± 80738* |
| MP-PD (19) | | | | | | | |
| Prophylactic | | | | | | | |
| E: OM-174- | 16744 | 3802 | 7411 | 21739 | 12500 | 21343 | 13923 ± 7362* |
| DP(1b) | | | | | | | |
| Therapeutic | | | | | | | |

*= p < 0.05 (Student t test)

Compared to the positive asthmatic group (B), animals treated with prophylactly with compounds 1b and 19 display about 8 times less BAL eosinophilia.

Moreover, when animals were treated three times therapeutically with compound 1b (group E), their eosinophil count was strongly decreased (by a factor of 64).

### B: Characterization of Pulmonary Cytokines

In a first series of analyzes, the results from each individual mouse, and the mean value of each group tested are shown in the table below:

### IL-4 and IL13 (Compounds 1b and 19)

IL-4 and IL-13 amounts were first analyzed in lungs of treated and untreated mice. Whereas IL-4 lung contents were very low to undetectable in PBS-challenged animals, IL-4 amounts increased 20-fold in LACK-challenged untreated control mice. Upon prophylactic treatment with OM-174-DP (1b) and OM-174-MP-PD (19), the amounts of IL-4 in lungs decreased by 6 and 4 fold, respectively (p<0.001 Mann&Whitney) (see table below). It should be noted that a similar reduction in IL-4 (4 fold compared to group B) was obtained by a therapeutic treatment with the synthetic molecule 1b (group E).

| | mice | | | | | | Mean IL-4 | SEM |
|---|---|---|---|---|---|---|---|---|
| Groups | Mice 1 | Mice 2 | Mice 3 | Mice 4 | Mice 5 | Mice 6 | (pg/ml) | IL-4 |
| A:1 untreated/PBS | 0.49 | 0.44 | 0.39 | NA | NA | NA | 0.44 | 0.05 |
| B:1 untreated/LACK | 9.88 | 4.51 | 10.47 | 6.31 | 11.27 | 9.54 | 8.66 | 2.65 |
| C:1 (1b) OM-174- | 1.1 | 3.33 | 0.71 | 2.43 | 1.23 | 0.17 | 1.50** | 1.17 |
| DP [SMORII-39] | | | | | | | | |
| Prophylactic | | | | | | | | |
| D: (19) OM-174- | 1.01 | 1.47 | 0.91 | 5.68 | 1.79 | 1.66 | 2.09** | 1.79 |
| MP-PD | | | | | | | | |
| [SMORII-32] | | | | | | | | |
| Prophylactic | | | | | | | | |
| E: (1b) OM-174- | 2.5 | 1 | 1.58 | 3.48 | 1.94 | 3.48 | 2.33** | 1.02 |
| DP | | | | | | | | |
| [SMORII-39] | | | | | | | | |
| Therapeutic | | | | | | | | |

**= p < 0.01 (Mann & Whitney) compared to the positive asthmatic group (B)

IL-13 was under the detection limit in PBS-challenged mice but was quantified as a mean at 50 pg/ml in asthmatic control mice (see table below, group B). As compared to these asthmatic mice, IL-13 amount was 4-fold reduced in OM-174-DP-prophylactly-treated mice (compound 1b) (p<0.001 Mann&Whitney), and 3-fold reduced in OM-174-MP-PD-treated mice (compound 19) (p<0.001) (see table below).

It should be noted that a similar reduction in IL-13 (3 fold compared to group B) was obtained by a therapeutic treatment with the synthetic molecule 1b (group E).

| | mice | | | | | | Mean IL13 | SEM |
|---|---|---|---|---|---|---|---|---|
| Groups | Mice 1 | Mice 2 | Mice 3 | Mice 4 | Mice 5 | Mice 6 | (pg/ml) | IL13 |
| A: untreated/PBS | 1.06 | 1.56 | 0.38 | NA | NA | NA | 1.00 | 0.59 |
| B: untreated/LACK | 50.83 | 26.62 | 55.06 | 42.91 | 62.21 | 65.98 | 50.60 | 14.32 |
| C: (1b) OM-174- | 8.39 | 25.39 | 8.2 | 24.29 | 12.52 | 1.59 | 13.40* | 9.54 |
| DP [SMORII-39] | | | | | | | | |
| Prophylactic | | | | | | | | |
| D: (19) OM-174- | 12.08 | 17.1 | 8.59 | 30.6 | 16.14 | 15.21 | 16.62* | 7.52 |
| MP-PD | | | | | | | | |
| [SMORII-32] | | | | | | | | |
| Prophylactic | | | | | | | | |
| E: (1b) OM-174- | 20.35 | 5.43 | 9.93 | 23.68 | 21.99 | 26.02 | 17.90* | 8.26 |
| DP | | | | | | | | |
| [SMORII-39] | | | | | | | | |
| Therapeutic | | | | | | | | |

*= p < 0.05 (Mann & Whitney) compared to the positive asthmatic group (B)

Additional Cytokines Measured after Three Therapeutic Administrations of Compound 1b

Since airway eosinophilia was so drastically reduced upon therapeutic treatment with the molecules tested, we sought, in a second series of analyses, to monitor additional cytokines (IL-5 and IFN-γ) in lung contents (for compound 1b only). The results are shown in the table below:

Indeed, when compared to lungs of untreated mice, the amounts of the Th2-cytokine: IL-5, were strongly reduced in lungs of treated mice. IL-5 amounts were reduced 2.8 fold upon treatment with OM-174-DP (compound 1b, p<0.01, Mann&Whitney).

| | mice | | | | | | Mean IL-5 | SEM |
|---|---|---|---|---|---|---|---|---|
| Groups | Mice 1 | Mice 2 | Mice 3 | Mice 4 | Mice 5 | Mice 6 | (pg/ml) | IL-5 |
| A: untreated/PBS | 0.25 | 0.25 | 0 | NA | NA | NA | 0.17 | 0.14 |
| B: untreated/LACK | 14.86 | 7.61 | 18.42 | 11.74 | 22.99 | 23.9 | 16.59 | 6.40 |
| E: (1b) OM-174- | 7.41 | 1.06 | 2.15 | 9.23 | 5.81 | 10.33 | 6.00** | 3.75 |
| DP | | | | | | | | |
| [SMORII-39] | | | | | | | | |
| Therapeutic | | | | | | | | |

Mann & Whitney, **p < 0.01

Clearly, compared to group B, a reduction in IL-5, a cytokine known to activate eosinophils, was obtained by a therapeutic treatment with the synthetic molecule 1b (group E).

This decrease was not due to a shift towards Th1 cytokine since IFN-γ levels were not enhanced (1.5 to 3 pg/ml) for all mice compared to group B animals (IFN-γ levels were even reduced twofold in compound 1b-treated mice, see table below):

| | mice | | | | | | Mean IFN-γ | SEM |
|---|---|---|---|---|---|---|---|---|
| Groups | Mice 1 | Mice 2 | Mice 3 | Mice 4 | Mice 5 | Mice 6 | (pg/ml) | IFN-γ |
| A: untreated/PBS | 0.51 | 0.56 | 0.45 | NA | NA | NA | 0.51 | 0.06 |
| B: untreated/LACK | 3.42 | 2.16 | 2.86 | 1.46 | 3.9 | 2.25 | 2.68 | 0.90 |
| E: (1b) OM-174- | 1.62 | 0.88 | 0.94 | 2.2 | 0.81 | 1.8 | 1.38* | 0.58 |
| DP | | | | | | | | |
| [SMORII-39] | | | | | | | | |
| Therapeutic | | | | | | | | |

Mann & Whitney, *p < 0.05

### C: Quantification of Seric Allergen-Specific IgE

In order to further characterize the immune status of mice after therapeutic treatment with OM-174-DP (compound 1b), we have analyzed LACK-specific IgE in sera of treated mice and in those of untreated mice by ELISA. The results from each individual mouse, and the mean value of each group tested are shown in the table below (therapeutic experiment with compound 1b).

| | mice | | | | | | Mean IgE | SEM |
|---|---|---|---|---|---|---|---|---|
| Groups | Mice 1 | Mice 2 | Mice 3 | Mice 4 | Mice 5 | Mice 6 | (ng/ml) | IgE |
| A: untreated/PBS | 84 | 32 | 133 | NA | NA | NA | 82.82 | 50.25 |
| B: untreated/LACK | 679 | 400 | 996 | 219 | 559 | 659 | 585 | 265.28 |
| E: (1b) OM-174- | 188 | 159 | 94 | 382 | 395 | 129 | 224.56* | 130.64 |
| DP | | | | | | | | |
| [SMORII-39] | | | | | | | | |
| Therapeutic | | | | | | | | |

Mann & Whitney, *p < 0.05

Whereas LACK-specific IgE levels increased 7-fold upon exposition to LACK aerosols, sera of OM-174-DP-treated mice contained 2.6-fold less LACK-specific IgE compared to untreated LACK challenged mice (p<0.05; Mann & Whitney).

### Conclusion:

In the first part of the present study, compounds (1b) and (19) were investigated for their effects when preventively administrated. It is shown that systemic administrations of these two synthetic compounds significantly affected the development of BAL eosinophilia. and significantly decreased IL-4 and IL-13 cytokines in lungs.

Moreover, it is shown here that the synthetic compounds of the invention display also a therapeutic anti-asthmatic potential, as illustrated by the results obtained with compound 1b (strong and significant decreases in allergen-induced BAL eosinophilia, pulmonary IL-4, IL-5, and IL-13, and decreased IgE levels).

In summary, the results presented in example 6 provide a clear example that the compounds of the invention could be clinically tested preventively of therapeutically against asthma.

### Example 7

### Effect of OM-174-DP (Compound 16), OM-174-MP-PD (Compound 19) OM-174-MP-EP (Compound 33), OM-174-MP-CM (Compound 35c) and OM-174-MP-PR (Compound 17) in an In Vitro Model of Compound 48/80-Stimulated Assay of Histamine Secretion

Using an in vitro rat model of mast cell degranulation (proposed by the CRO CEREP, catalog number 2006: 771-c), we aimed to investigate whether the synthetic molecules OM-174-DP (compound 1b) OM-174-MP-PD (19), OM-174-MP-EP (33), OM-174-MP-CM (35c) and OM-174-MP-PR (17) would inhibit histamine secretion by compound 48/80-stimulated mastocytes. The protocol used is briefly summarized below:

### Protocol:

General Procedure

| | | Reference | |
|---|---|---|---|
| Assay | Origin | Compound | Bibliography |
| Histamine secretion | rat mast cells | SCG | ²⁹Hakanson |
| (compound 48/80-stimulated) | | | et al. |

Experimental Conditions

| | | | Reaction | Method of |
|---|---|---|---|---|
| Assay | Stimulus | Incubation | Product | Detection |
| Histamine secretion | compound | 2 min./ | histamine | Fluorimetry |
| (compound 48/ | 48/80 | 37° C. | | |
| 80-stimulated) | (0.1 μg/ml) | | | |

### Analysis and Expression of the Results

The results are expressed as a percent of control specific activity ((measured specific activity/control specific activity)×100) obtained in the presence of the test compounds.

The IC50 values (concentration causing a half-maximal inhibition of control specific activity) were determined by non-linear regression analysis of the inhibition curves generated with mean replicate values using Hill equation curve fitting (Y=D+[(A−D)/(1+(C/C50)nH)], where Y=specific activity, D=minimum specific activity, A=maximum specific activity, C=compound concentration, C50IC50, and nH=slope factor). This analysis was performed using a software developed at Cerep (Hill software) and validated by comparison with data generated by the commercial software SigmaPlot® 4.0 for Windows® (© 1997 by SPSS Inc.).

### Compounds Tested:

| | | Molecular |
|---|---|---|
| Compound tested | Batch Number | weight |
| OM-174-DP (1b) | SMORII- | 1134 |
| | 69_110906 | |
| OM-174-MP-PD (19) | SMORII- | 1128 |
| | 32_050906 | |
| OM-174-MP-EP (33) | SMOR-II-83 | 1179 |
| OM-174-MP-CM | SMORII-135 | 1113 |
| (35c) | | |
| OM-174-MP-PR (17) | KAS1-108 | 1097 |

### Reference Compound

In each experiment, the reference compound was tested concurrently with the test compounds in order to assess the assay suitability. It was tested at several concentrations (for IC₅₀ value determination), and the data were compared with historical values determined at CEREP. The assay was rendered valid if the suitability criteria were met, in accordance with the corresponding Standard Operating Procedure.

### Results:

The IC₅₀ values determined for the test compounds and the reference (5 different tests) are indicated in the table below. The IC₅₀ values for the reference compound are within accepted limits of the historic average obtained at CEREP.

| Compounds tested | IC50 |
|---|---|
| OM-174-DP (1b) | 8.6E−08M |
| OM-174-MP-PD (19) | 1.6E−07M |
| OM-174-MP-EP (33) | 2.1E−07M |
| OM-174-MP-CM | 3.2E−07M |
| (35c) | |
| OM-174-MP-PR (17) | 1.6E−06M |
| SCG (ref 1) | 4.7E−06M |
| SGC (ref 2) | 1.1E−06M |
| SGC (ref 3) | 1.3E−05M |
| SCG (ref 4) | 2.3E−05M |
| SCG (ref 5) | 9.5E−07M |
| Mean reference | 8.6E−06M |

### Conclusion:

Clearly the drugs tested are potential inhibitors of histamine secretion induced by compound 48/80-stimulated mast cells. They are all more active than the reference tested: SGS.

### Example 8

### Effect of OM-174-MP-PD (Compound 19) OM-174-MP-EP (Compound 33), OM-174-MP (Compound 16) and OM-174-MP-PR (Compound 17) in In Vitro Cellular Models Expressing Human Toll-Like Receptors (TLRs)

The chemical structure and origin of the products of the invention (OM-174-DP was originally obtained from degradated LPS from E. coli) may suggest that the drugs may be active via TLRs receptors, and more specifically via TLR4 and TLR2. TLR receptors are expressed principally (but not exclusively) by immune cells such as monocytes, macrophages, dendritic cell, T-cells etc, and are key sensors of microbial products, which can be recognized as signal dangers by the host. Even-though they trigger first an unspecific innate immunity, TLR activation will initiate a full immunological cascade, which will result, in the presence of antigens, to the development of acquired immunity.

Cells that constitutively express a given functional TLR gene are valuable tools for many applications, such as the study of the mechanisms involved in TLR recognition or signaling, and the development of new potential therapeutic drugs. It was therefore the aim of the experiments described below to test the activity of 4 compounds of the invention on these key adaptors of the immune response.

The Responses were Tested in the Following Cellular Systems:
a) THP1 blue (lecture of optical density at 625 nm after 48 hours)
b) HEK-TLR2 (IL-8 ELISA after 24 hours)
c) HEK-TLR2-CD14 (IL-8 ELISA after 24 hours)
d) HEK-MD2-TLR4-CD14 (IL-8 ELISA after 24 hours)

### a) THP-1 Blue

A first series of experiment was performed on THP-1 cells, which express naturally both TLR2 and TLR4.

THP-1 cells are human peripheral blood monocytic cells. Monocytes play a key role in innate immunity and express most TLRs at various levels. As for the primary cells, THP-1 cells activate NF-κB and other transcription factors in response to TLR ligands

In contrast to HEK293 cells that were engineered to respond to specific TLR agonists (see below), THP-1 cells naturally express the TLR genes and all the genes involved in the signaling cascade.

To facilitate analysis of TLR response in monocytes, InvivoGen (Toulouse, France) provides THP-1 clones stably transfected with an NF-κB-inducible reporter system, called THP1-Blue™. THP-1 Blue cells were stably transfected with a reporter plasmid expressing a secreted embryonic alkaline phosphatase (SEAP) gene under the control of a promoter inducible by several transcription factors such as NF-κB and AP-1. Upon TLR stimulation, THP1-Blue™ cells activate transcription factors and subsequently the secretion of SEAP which is easily detectable when using QUANTI-Blue\u2122a medium that turns purple/blue in the presence of SEAP.

The cells were stimulated with the controls and compounds of the invention according to manufacturer's instructions.

Results: Increased OD at 625 nm after 48 Hours:

The results for the controls (negative=LPS K12CD25 ultrapure, a TLR4 agonist; and PAM3CSK4=positive, a TLR2 agonist) are provided in the table below. The results (expressed as OD of arbitrary units) show the mean values (from duplicate measures) of Optical density read at 625 nm, 48 h after stimulation at 37° C. with the controls (up to 1000 ng/ml):

### Controls:

| Controls | | PAM |
|---|---|---|
| ng/ml | LPS Mean | Mean |
| 1000 | 1.00 | 1.35 |
| 333 | 0.97 | 1.38 |
| 111 | 0.69 | 1.30 |
| 37 | 0.46 | 1.22 |
| 12 | 0.27 | 1.11 |
| 4 | 0.16 | 0.85 |
| 1 | 0.13 | 0.48 |
| 0 | 0.10 | 0.11 |

The cell line responds clearly to both TLR2 and TLR4 agonists, PAM3CSK4 and LPS K12CD25 ultrapure respectively.

Then the following four compounds of the invention were tested in this assay OM-174-MP-PD (compound 19) OM-174-MP-EP (compound 33), OM-174-MP (compound 16) and OM-174-MP-PR (compound 17).

The results (mean of duplicate OD values at 625 nm after 48 h) are shown below:

### Compounds:

| Compounds/ | Mean | Mean | Mean | Mean |
|---|---|---|---|---|
| dose | (compound | (compound | (compound | (compound |
| (ng/ml) | 16) | 19) | 33) | 17) |
| 10000 | 0.22 | 0.77 | 0.35 | 1.15 |
| 3333 | 0.18 | 0.58 | 0.25 | 1.02 |
| 1111 | 0.17 | 0.37 | 0.19 | 0.74 |
| 370 | 0.16 | 0.24 | 0.16 | 0.36 |
| 124 | 0.15 | 0.17 | 0.13 | 0.19 |
| 41 | 0.13 | 0.13 | 0.12 | 0.15 |
| 14 | 0.15 | 0.15 | 0.13 | 0.13 |
| 0 | 0.14 | 0.14 | 0.13 | 0.14 |

Compounds (19) and (17) are good activators of THP-1 cells, whereas the activity of compounds (16) and (33) is weaker.

As the compounds of the invention are active on a system which expresses both TLR2 and TLR4, we then checked their activity on HEK cells expressing only either TLR2, or only TLR4, but not both TLRs simultaneously.

### b) HEK-TLR2

The HEK293 cell line was chosen for its null or low basal expression of the TLR genes. These cells enable efficient monitoring of TLR activity using ELISA analysis such as IL-8 titration or reporter-based systems that monitor TLR-induced NF-κB activation.

HEK-TLR2 cells (Invivogen, Toulouse, France) are engineered HEK293 cells stably transfected with multiple genes from the TLR2 pathway that include TLR2 and genes participating in the recognition or involved in the signaling cascade. These cells secrete IL-8 after TLR2 stimulation. The experiments were performed according to manufacturers' instructions.

Briefly, 2×10⁴ cells/well (200 ul RPMI) are incubated at 37° C. during 3 days (5% CO₂). The medium is removed, and 90 ul RPMi+5% FCS is added to the wells Then the agonists and controls are added (10 ul/well). The cells return to the incubator for 24 hours. The supernatants are collected and the IL-8 ELISA is performed according to manufacturer's instructions

### Results: IL-8 Secretion

The results for the controls (negative=LPS K12CD25 ultrapure, a TLR4 agonist; and PAM3CSK4=positive, a TLR2 agonist) are provided in the table below. The results (expressed as pg/ml of IL-8) show the mean values (from duplicate measures) of IL-8 secretion, 24 h after stimulation with the controls (up to 1000 ng/ml):

### Controls:

| Controls | | PAM |
|---|---|---|
| ng/ml | LPS Mean | Mean |
| 1000 | 0.90 | 32.94 |
| 333 | 0.41 | 28.24 |
| 111 | 0.30 | 23.93 |
| 37 | 0.23 | 18.33 |
| 12 | 0.19 | 9.12 |
| 4 | 0.23 | 3.89 |
| 1 | 0.29 | 1.84 |
| 0 | 0.28 | 0.38 |

The cell line responds clearly only to the TLR2 agonist PAM3CSK4.

Then the following four compounds of the invention were tested in this assay OM-174-MP-PD (compound 19) OM-174-MP-EP (compound 33), OM-174-MP (compound 16) and OM-174-MP-PR (compound 17).

The results (mean of supplicate values of IL-8 secreted in pg/ml after 24 h) are shown below:

### Compounds:

| Compounds/ | Mean | Mean | Mean | Mean |
|---|---|---|---|---|
| dose | (compound | (compound | (compound | (compound |
| (ng/ml) | 16) | 19) | 33) | 17) |
| 10000 | 0.27 | 0.62 | 3.64 | 2.02 |
| 3333 | 0.19 | 0.37 | 1.66 | 0.88 |
| 1111 | 0.15 | 0.27 | 0.68 | 0.39 |
| 370 | 0.12 | 0.20 | 0.31 | 0.25 |
| 124 | 0.15 | 0.16 | 0.23 | 0.24 |
| 41 | 0.09 | 0.15 | 0.18 | 0.20 |
| 14 | 0.07 | 0.06 | 0.12 | 0.19 |
| 0 | 0.18 | 0.12 | 0.17 | 0.23 |

Clearly compounds 33 and 17 are able to activate IL-8 secretion via TLR2.

### c) HEK-TLR2-CD14

These preliminary results were confirmed in another cell line expressing concomitantly TLR2 and CD14. The procedure used is similar to the one described just above.

The results for the controls and the 4 compounds tested are shown in the 2 tables below:

### Results: IL-8 Secretion

The results for the controls (negative=LPS K12CD25 ultrapure, a TLR4 agonist; and PAM3CSK4=positive, a TLR2 agonist) are provided in the table below. The results (expressed as pg/ml of IL-8) show the mean values (from duplicate measures) of IL-8 secretion, 24 h after stimulation with the controls (up to 1000 ng/ml):

### Controls:

| Controls | | PAM |
|---|---|---|
| ng/ml | LPS Mean | Mean |
| 1000 | 0.67 | 39.04 |
| 333 | 0.32 | 33.30 |
| 111 | 0.19 | 22.64 |
| 37 | 0.13 | 15.90 |
| 12 | 0.16 | 6.78 |
| 4 | 0.11 | 2.55 |
| 1 | 0.11 | 1.33 |
| 0 | 0.22 | 0.32 |

As for the HEK-TLR2 cell line, HEK-TLR2-CD14 cells respond also clearly only to the TLR2 agonist PAM3CSK4.

### Compounds:

Then the following four compounds of the invention were tested in this HEK-TLR2-CD14 assay:

OM-174-MP-PD (compound 19) OM-174-MP-EP (compound 33), OM-174-MP (compound 16) and OM-174-MP-PR (compound 17).

The results (mean of supplicate values of IL-8 secreted in pg/ml after 24 h) are shown below:

| Compounds/ | Mean | Mean | Mean | Mean |
|---|---|---|---|---|
| dose | (compound | (compound | (compound | (compound |
| (ng/ml) | 16) | 19) | 33) | 17) |
| 10000 | 0.22 | 0.65 | 4.24 | 3.59 |
| 3333 | 0.13 | 0.26 | 1.49 | 1.06 |
| 1111 | 0.06 | 0.17 | 0.52 | 0.40 |
| 370 | 0.01 | 0.10 | 0.19 | 0.22 |
| 124 | 0.03 | 0.11 | 0.17 | 0.20 |
| 41 | 0.00 | 0.03 | 0.03 | 0.14 |
| 14 | 0.03 | 0.05 | 0.10 | 0.13 |
| 0 | 0.12 | 0.11 | 0.17 | 0.19 |

The results confirm those obtained with the HEK-TLR2 cell line: compounds 33 and 17 are able to activate IL-8 secretion via TLR2.

### d) HEK-MD2-TLR4-CD14

TLR4 is extensively studied as it is the major receptor involved in the recognition of lipopolysaccharide (LPS) responsible for septic shock.

HEK-MD2-TLR4-CD14 are highly sensitive to LPS. They were obtained by stable transfection of HEK293 cells with the TLR4, MD2 and CD14 genes and an NF-κB-inducible reporter system. They secrete IL-8.

We used the same experimental procedure as for the other HEK cell lines described above.

The results for the controls and the 4 compounds tested are shown in the 2 tables below:

### Results: IL-8 Secretion

The results for the controls (positive=LPS K12CD25 ultrapure, a TLR4 agonist; and PAM3CSK4=negative, a TLR2 agonist) are provided in the table below. The results (expressed as pg/ml of IL-8) show the mean values (from duplicate measures) of IL-8 secretion, 24 h after stimulation with the controls (up to 1000 ng/ml):

### Controls:

| Controls | | PAM |
|---|---|---|
| ng/ml | LPS Mean | Mean |
| 1000 | 58.43 | 2.17 |
| 333 | 56.58 | 1.87 |
| 111 | 58.20 | 1.83 |
| 37 | 50.24 | 1.72 |
| 12 | 32.15 | 1.76 |
| 4 | 18.08 | 1.78 |
| 1 | 10.04 | 1.75 |
| 0 | 1.87 | 1.97 |

As expected, the cell line HEK-MD2-TLR4-CD14 responds clearly only to the TLR4 positive control ultrapure LPS-K12, but not to the negative TLR2 control agonist PAM3CSK4.

### Compounds:

Then the following four compounds of the invention were tested in this HEK-MD2-TLR4-CD14 assay:

OM-174-MP-PD (compound 19) OM-174-MP-EP (compound 33), OM-174-MP (compound 16) and OM-174-MP-PR (compound 17).

The results (mean of supplicate values of IL-8 secreted in pg/ml after 24 h) are shown below:

| Compounds/ | Mean | Mean | Mean | Mean |
|---|---|---|---|---|
| dose | (compound | (compound | (compound | (compound |
| (ng/ml) | 16) | 19) | 33) | 17) |
| 10000 | 1.90 | 1.96 | 1.78 | 1.70 |
| 3333 | 1.63 | 1.88 | 1.62 | 1.89 |
| 1111 | 1.60 | 1.73 | 1.58 | 1.94 |
| 370 | 1.66 | 1.67 | 1.64 | 1.85 |
| 124 | 1.62 | 1.56 | 1.65 | 1.93 |
| 41 | 1.68 | 1.64 | 1.79 | 1.97 |
| 14 | 1.75 | 1.69 | 1.83 | 1.88 |
| 0 | 1.88 | 1.84 | 1.88 | 2.11 |

Clearly, none of the compounds tested is active in this TLR4 assay.

General Conclusion on these TLR Assays:

Very interestingly, the results obtained show that the compounds of the invention are active on human cells expressing preferentially the human TLR2 receptor.

### REFERENCES

¹ (a) Davies, J. G.; Bauer. J.; Hirt, P.; Schulthess, A. PCT 9514026 (Chem. Abstr. 124, 9326). (b) Brandenburg, K.; Lindner, B.; Schromm, A.; Koch, M. H. J.; Bauer, J.; Merkli, A.; Zbaeren, C.; Davies, J. G.; Seydel, U. Eur. J. Biochem. 2000, 267, 3370-3377.
² (a) Zähringer, U.; Lindner, B.; Rietschel, T. H. Adv. Carbohydr. Chem. Biochem. 1994, 50, 211-276. (b) Seydel, U.; Schromm, A. B.; Blunck, R.; Brandenburg, K. Chem. Immunol. 2000, 74, 5-24.
³ Johnson, A. G. Clin. Microbiol. Rev. 1994, 7, 277-289.
⁴ Alexander, C.; Rietschel, E. T. J. Endotoxin Res. 2001, 7, 167-202.
⁵ (a) Raetz, C. R. H. in ‘E. coli and Salmonella: Cellular and Molecular Biology’. Neidhardt, F. C. ed.; American Society for Microbiology, Washington, D.C., 1996, 1035-1063. (b) Rietschel, E. T.; Brade, H.; Holst, O.; Brade, L.; Müller-Loennies, S.; Mamat, U.; Zähringer, U.; Beckmann, F.; Seydel, K.; Brandenburg, K.; Ulmer, A. J.; Mattern, T.; Heine, H.; Schletter, J.; Hauschildt, S.; Loppnow, H.; Schoenbeck, U.; Flad, H.-D.; Schade, U. F.; Di Padova, F.; Kusumoto, S.; Schumann, R. R. Curr. Top. Microbiol. Immunol. 1996, 216, 39-81.
⁶ (a) Imoto M., Kusumoto S., Shiba T., Naoki H., Iwashita T., Rietschel E. Th., Wollenweber H.-W, Galanos C. and Lüderitz O., Tetrahedron Letters, 1983, 24, 4017. (b) Imoto M., Kusumoto S., Shiba T., Rietschel E. Th., Galanos C. and Lüderitz O., Tetrahedron Letters, 1985, 26, 907-908.
⁷ Ribi E.; Mar. 13, 1984, U.S. Pat. No. 4,436,727.
⁸ Myers K. R., Truchot A., Mar. 27, 1990, U.S. Pat. No. 4,912,094.
⁹ Ulrich J. T., Myers K. R., Vaccine Design: The subunit and Adjuvant Approach; Powell M:F:, Newman M:J:, Eds.; Plenum: New York, 1995, 495-524.
¹⁰ Onier, N.; Hilpert, S.; Arnould, L.; Saint-Giorgio, V.; Davies, J. G.; Bauer, J.; Jeannin, J.-F. Clin. Exp. Metastasis 1999, 17, 299-306.
¹¹ Kusomoto, S.; Yoshimura, H.; Imoto, M.; Shimamoto, T.; Shiba, T. Tetrahedron Letters, 1985, 26, 909-912
¹² Imoto, M.; Yoshimura, H.; Shimamoto, T.; Sakaguchi, N.; Kusomoto, S.; Shiba, T. Bull. Soc. Chim. Jpn. 1987, 60, 2205-2214.
¹³ Imoto, M.; Yoshimura, H.; Yamamoto, M.; Shimamoto, T.; Kusomoto, S.; Shiba, T. Bull. Soc. Chim. Jpn. 1987, 60, 2197-2204.
¹⁴ (a) Kusama, T.; Soga, T.; Shioya, E.; Nakayama, K.; Nakajima, H.; Osada, Y.; Ono, Y.; Kusumoto, S.; Shiba, T. Chem. Pharm. Bull. 1990, 38, 3366-3372. (b) Kusama, T.; Soga, T.; Ono, Y.; Kumazawa, E.; Shioya, E.; Osada, Y.; Kusumoto, S.; Shiba, T. Chem. Pharm. Bull. 1991, 39, 1994-1999.
¹⁵ Oikawa, M.; Wada, A.; Yoshizaki, H.; Fukase, K.; Kusumoto, S.; Bull. Soc. Chim. Jpn. 1997, 70, 1435-1440.
¹⁶ (a) Fukase, K.; Liu, W-C.; Suda, Y.; Oikawa, M.; Wada, A.; Mori, S.; Ulmer, A. J.; Rietschel, E. Th.; Kusumoto, S.; Tetrahedron. Letters 1995, 36, 7455-7458; (b) Liu, W-C.; Oikawa, M.; Fukase, K.; Suda, Y.; Winarmo, H.; Mori, S.; Hashimoto, M.; Kusumoto, S.; Bull. Soc. Chim. Jpn. 1997, 70, 1441-1450; (c) Fukase, K.; Fukase, Y.; Oikawa, M.; Liu, W-C.; Suda, Y.; Kusumoto, S.; Tetrahedron. 1998, 54, 4033-4050
¹⁷ Liu, W-C.; Oikawa, M.; Fukase, K.; Suda, Y.; Kusumoto, S.; Bull. Soc. Chim. Jpn. 1999, 72, 1377-1385.
¹⁸ (a) Sakai, Y.; Oikawa, M.; Yoshizaki, H.; Ogawa, T.; Suda, Y.; Fukase, K.; Kusumoto, S.; Tetrahedron Letters, 2000, 41, 6843-6847. (b) Sakai, Y.; Oikawa, M.; Kusumoto, S; Ogawa, T.; Jpn. Kokai Tokkyo Koho, 2000, J.P. Patent 2000226397.
¹⁹ Zamyatina, A.; Sekljic, H.; Brade, H.; Kosma, P. Tetrahedron 2004, 60, 12113-12137.
²⁰ (a) Jiang, Z.-H.; Bach, M. V.; Budzynski, W. A.; Krants, M. J.; Koganty, R. R.; Longenecker, B. M. Bioorg. Med. Chem. Lett., 2002, 12, 2193-2196; (b) Jiang, Z.-H.; Bach, M. V.; Yalamati, D.; Koganty, R. R.; Longenecker, B. M.; May 25, 2001; PCT WO 01/36433.
²¹ Ogawa, T.; Asai, Y.; Yamamoto, H.; Taiji, Y.; Jinno, T.; Kodama, T.; Niwata, S.; Shimauchi, H.; Ochiai, K.; FEMS Immunology and Medical Microbiology, 2000, 28, 273-281.
²² Qureshi N.; Honovich J. P.; Hara H.; Cotter R. J.; Takayama K. J.; J. Biol. Chem., 1988, 263, 5502-5504.
²³ Christ W. J.; McGuinness P. D.; Asano O.; Wang Y.; Mullarkey M. A.; Perez M.; Hawkins L. D.; Blythe T. A.; Dubuc G. R.; Robidoux A. L; J. Am. Chem. Soc. 1994, 116, 3637-3638.
²⁴ Christ W. J.; Rossignol D. P.; Kobayashi S.; Kawata T.; Aug. 10, 1999; U.S. Pat. No. 5,935,938.
²⁵ (a) Watanabe, Y.; Mochizuki, T.; Shiozaki, M.; Kanai, S.; Kurakata, S. I.; Nishijima, M.; Carbohydr. Res., 2001, 333, 203-231; (b) Shozaki, M.; Watanabe, Y.; Iwano, Y.; Kaneko, T.; Doi, H.; Tanaka, D.; Shimozato, T.; Kurakata, S.-I.; Tetrahedron, 2005, 61, 5101-5122.
²⁶ Burns E, Bachrach G, Shapira L, Nussbaum G. Cutting Edge: TLR2 is required for the innate response to Porphyromonas gingivalis: activation leads to bacterial persistence and TLR2 deficiency attenuates induced alveolar bone resorption. J Immunol. 2006 Dec. 15; 177(12):8296-300.
²⁷ Samsom J, Kraal G, Lauener R, Chiavaroli C, Bauer J, Davies W. The novel adjuvant OM-174 activates murine bone marrow dendritic cells through TLR-4 and can also signal through human TLR2. The 5th Annual Conference on Vaccine Research. Baltimore May 6-8, 2002.
²⁸ Garay R P, Viens P, Bauer J, Normier G, Bardou M, Jeannin J F, Chiavaroli C. Cancer relapse under chemotherapy: why TLR2/4 receptor agonists can help. Eur J. Pharmacol. 2007 Jun. 1; 563(1-3):1-17.
²⁹ Håkanson R, Rönnberg A L, Sjölund K. Anal Biochem. 1972 June; 47(2):356-70. Fluorometric determination of histamine with OPT: optimum reaction conditions and tests of identity.

1-92. (canceled)

93. A process for the preparation of an asymmetrically or symmetrically substituted β-(1→6)-linked glucosamine comprising reacting a compound of the formula 10: 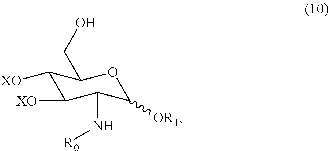
wherein:
R₁ is a group selected from a (C₃-C₆) alkenyl, such as a C₃ or C₄ alkenyl, preferably 2-propenyl or 1-propenyl;
X is a hydrogen, a group selected from benzyl or a substituted benzyl, such as 4-methoxybenzyl or 3,4-dimethoxybenzyl or 2,5-dimethoxybenzyl or 2,3,4-trimethoxybenzyl or 3,4,5-trimethoxybenzyl;
R₀ is selected from R₅ or R₂, wherein R₅ is selected from:
(i) an acyl group derived from a, straight chain-carboxylic acid having from 2 to 24 carbon atoms, preferably a hydroxy acyl group, such as a 3-hydroxy acyl group, an oxo acyl group such as a 3-oxo acyl group, an amino acyl group such as a 3-amino acyl group;
(ii) an acyloxyacyl group, preferably a 3-acyloxyacyl group, an acylaminoacyl group, preferably a 3-acylaminoacyl group, an acyl thioacyl group, preferably a 3-acylthioacyl group;
(iii) an alkyloxyacyl group, preferably a (C₂-C₂₄) alkyloxyacyl group, an alkenyloxyacyl group, preferably a (C₂-C₂₄) alkenyloxyacyl group, an alkynyloxyacyl group, preferably a (C₂-C₂₄) alkynyloxyacyl group an alkyl aminoacyl group, preferably a (C₂-C₂₄) alkylaminoacyl group, an alkenylaminoacyl group, preferably a (C₂-C₂₄) alkenylaminoacyl group, an alkynylaminoacyl group, preferably a (C₂-C₂₄) alkynylaminoacyl group, an alkylthioacyl group, preferably a (C₂-C₂₄) alkylthioacyl group, an alkenylthioacyl group, preferably a (C₂-C₂₄) alkenylthioacyl group, an alkynylthioacyl group, preferably a (C₂-C₂₄) alkynylthioacyl group, an acyl group derived from a branched chain-carboxylic acid having from 2 to 48 carbon atoms, preferably a carboxylic acid branched at the 3-position;
wherein in the groups (i), (ii), (iii) the hydrocarbon chain of the acyl may be saturated or unsaturated and the hydrocarbon chain of the acyl, alkyl, alkenyl, alkynyl may be branched or straight and optionally may be substituted with one or more groups independently selected from halogen such as fluoro, chloro, bromo, or iodo; a hydroxyl or hydroxyl derivative —OY, wherein Y is as defined below; an amine or amine derivative —NHW, wherein W is as defined below; a group —OZ, wherein Z is selected from (f), (g), (h), (i), (j), (k) as defined below;
and R₂ is a group selected from a (C₁-C₆) halogenated alkoxy carbonyl, such as 2,2,2-trichloroethoxycarbonyl (TROC) or a 1,1-dimethyl-2,2,2-trichloroethoxycarbonyl (TCBOC);
with a compound of the formula 7: 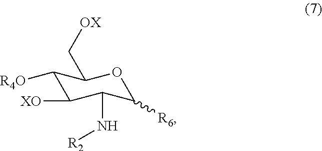
wherein R₄ is selected from:
(a) an acyl group as defined in (i), (ii) or (iii) for R₅; a branched or straight alkyl group, preferably a branched or straight (C₁-C₂₄) alkyl group; a branched or straight alkenyl group, preferably a branched or straight (C₁-C₂₄) alkenyl group; a branched or straight alkynyl group, preferably a branched or straight (C₁-C₂₄) alkynyl group;
(b) a group —[(C₁-C₂₄) alkyl]-COOX, —[(C₂-C₂₄) alkenyl]-COOX or —[(C₂-C₂₄) alkynyl]-COOX wherein X is as defined above;
(c) a group —[(C₁-C₂₄) alkyl]-NHW, —[(C₁-C₂₄) alkenyl]-NHW or —[(C₁-C₂₄) alkynyl]-NHW wherein W is as defined below;
(d) a formyl alkyl group, preferably a formyl [(C₁-C₂₄) alkyl] group; a formyl alkenyl group, preferably a formyl [(C₁-C₂₄) alkenyl] group; a formyl alkynyl group, preferably a formyl [(C₁-C₂₄) alkynyl] group;
(e) a dimethoxyphosphoryl group;
(f) a group —P(O)(OY)₂, wherein Y is as defined below;
(g) a group —P(O)(OH)—O[(C₁-C₂₄) alkyl]-NHW, —P(O)(OH)—O[(C₁-C₂₄) alkenyl]-NHW or —P(O)(OH)—O[(C₁-C₂₄) alkynyl]-NHW wherein W is as defined below;
(h) a group —P(O)(OH)—O[(C₁-C₂₄) alkyl], —P(O)(OH)—O[(C₁-C₂₄) alkenyl], or —P(O)(OH)—O[(C₁-C₂₄) alkynyl];
(i) a group —P(O)(OH)—O[(C₁-C₂₄) alkyl]-COOX, —P(O)(OH)—O[(C₁-C₂₄) alkenyl]-COOX, —P(O)(OH)—O[(C₁-C₂₄) alkynyl]-COOX, wherein X is as defined above;
(j) a group —S(O)(OH)₂;
(k) a protective group selected from benzyl or a substituted benzyl, such as 4-methoxybenzyl or 3,4-dimethoxybenzyl or 2,5-dimethoxybenzyl or 2,3,4-trimethoxybenzyl or 3,4,5-trimethoxybenzyl; or from a (C₃-C₆) alkenyl, such as a C₃ or C₄ alkenyl, preferably 2-propenyl or 1-propenyl;
wherein alkyl, alkenyl, alkynyl groups may be branched or straight and may be unsubstituted or optionally are substituted with one or more groups independently selected from halogen such as fluoro, chloro, bromo, or iodo; a hydroxyl or hydroxyl derivative —OY, wherein Y is as defined below; an amine or amine derivative —NHW, wherein W is as defined below; or a group —OZ, wherein Z is selected from (f), (g), (h), (i), (j), (k);
and wherein Y is selected from hydrogen; an (C₃-C₆) alkenyl, such as a C₂ or C₃ alkenyl, preferably 2-propenyl or 1-propenyl group; a group selected from benzyl or a substituted benzyl, such as 4-methoxybenzyl or 3,4-dimethoxybenzyl or 2,5-dimethoxybenzyl or 2,3,4-trimethoxybenzyl or 3,4,5-trimethoxybenzyl; a O-Xylylene group;
and wherein W is selected from hydrogen; a benzyloxycarbonyl group or a 9-fluorenylmethyloxycarbonyl;
and wherein R₆ is a group selected from trichloroacetimidate, fluoride, chloride, bromide, and X and R₂ are as defined above, under reaction conditions suitable for forming a compound of the formula 11h: 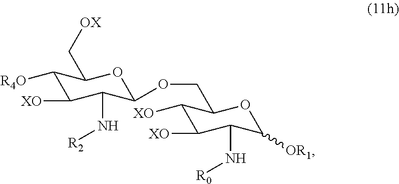
wherein R₁, R₂, R₄, R₀ and X are as defined above, which process optionally further comprises one or more of the following steps (2)-(11)
(2) Reacting the compound of the formula 11h under reaction conditions suitable for the removal of a number of groups R₂ of said compound of the formula 11h, while forming a compound of the formula 12a: 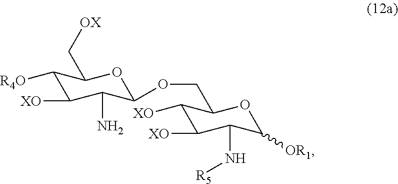
wherein R₁, R₄, R₅ and X are as defined above, when R₀ is selected as R₅ in formula 11h, or a compound of the formula 12b: 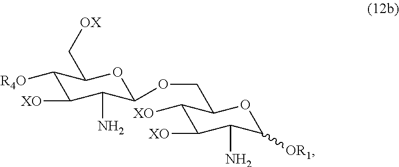
wherein R₁, R₄, and X are as defined above, when R₀ is selected as R₂ in formula 11h.
(3) reacting the compound of the formula 12a or 12b under reaction conditions suitable for forming an amide bond between a free amino group of said compound of the formula 12a or 12b and a carboxy group of a (activated) carboxylic acid of the formula R₅OH, wherein R₅ is as defined before, to form of a compound of the formula 13: 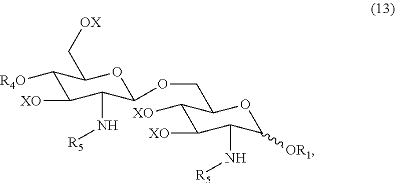
wherein R₁, R₄, R₅, and X are as defined above
(4) Forming a hemiacetal of the formula 14: 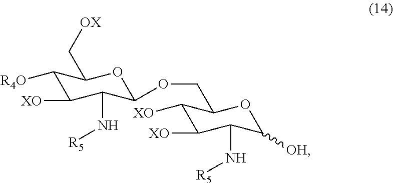
wherein R₄, R₅, and X are as defined above, by removal under suitable reaction conditions of the group R₁ from the compound of the formula 13, as defined above;
(5) Phosphorylation of the free hydroxyl group of compound 14, under reaction conditions suitable for forming a compound of the formula 15a: 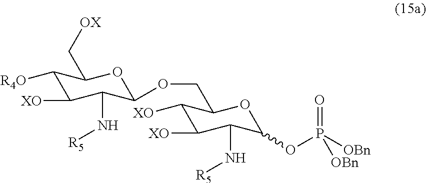
(6) Sulfatation of the free hydroxyl group of compound 14, under reaction conditions suitable for forming a compound of formula 15b: 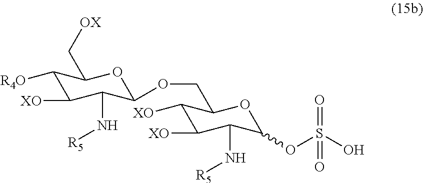
(7) Reacting the free hydroxyl group of compound 14 with an (activated) carboxylic acid of the formula R₈OH, wherein R₈ is selected from (a) as defined previously for R₄, to form compound of the formula 15c: 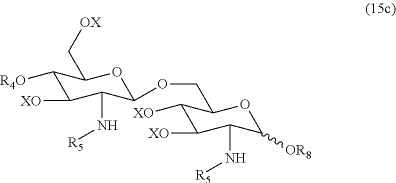
wherein R₄, R₅, and X are as defined before, and R₈ is selected from (a) as defined previously for R₄.
(8) Coupling of leaving group such as a trichloroacetimidate group to the free hydroxyl group of compound 14, under reaction conditions suitable for forming a compound of the formula 24: 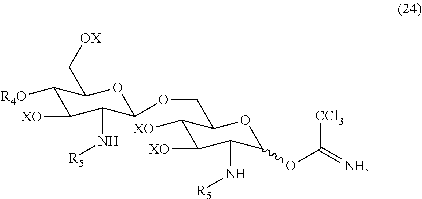
wherein R₄, R₅, and X are as defined previously.
(9) Reacting Compound 24 of step (8) with an organic molecule R₈OH, wherein R₈ is selected from (b), (c), (d) or (e) as defined previously for R₄, under reaction conditions suitable for forming a compound of the formula 15d: 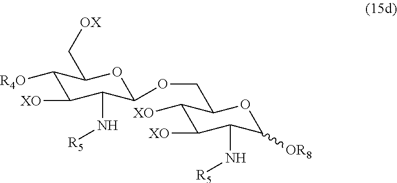
(10) Reacting, under suitable reaction conditions, reactive groups, such as hydroxyl groups, amine groups, carboxy groups, or carbon double bonds, on a compound of the formula 15a, 15b, 15c, 15d or 13 for example in a reaction selected from esterification, methylation, amidation, oxidation, hydrogenation or α, β hydroxylation with osmium tetroxide, and wherein said reacting of reactive groups optionally is preceded by removing protective groups, such as the groups Y or W to liberate said reactive groups;
(11) Removing a number of protecting groups X from a compound of the formula 13, 14, 15a, 15b, 15c or 15d, under reaction conditions suitable for forming of a compound of the formula 1: 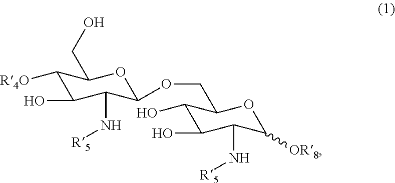
wherein R₄′, R₅′ and R₇′ are as defined previously for R₄, R₅ and R₇ respectively, and R₈′ is selected from (a), (b), (c), (d), (e), (f), (g), (h), (i) (j) or (k) as defined previously for R₄, or is selected as H, and wherein Y and W preferably are H

94. A process according to claim 93, wherein the reaction between compounds of formula 10 and 7 is carried out in the presence of a solvent.

95. A process according to claim 94, wherein the solvent is dichloromethane.

96. A process according to claim 93, wherein the reaction between compounds of formula 10 and 7 is carried out in the presence of a catalytic amount of Lewis acid.

97. A process according to claim 96, wherein the Lewis acid is trimethylsilyltrifluoromethanesulfonate.

98. A process according to claim 93 wherein compounds of formula 10, 7, 11h, 12a, 12b, 13, 14, 15a, 15b, 15c, 24, 15d, 1 are prepared and/or used as α anomer, β anomer, stereoisomers, or mixtures thereof.

99. A process according to claim 93, wherein when R₅ is acyl, it is preferably substituted at the 3-position.

100. A process according to claim 99 wherein the 3-position substituted acyl is selected from 3-acyloxyacyl, 3-acylaminoacyl or 3-acylthioacyl group.

101. A process according to claim 100 wherein the 3-acyloxyacyl preferably comprises a 3-hydroxy fatty acid residue or for the ester-linked group, a 3-oxo fatty acid residue.

102. A process according to claim 93, wherein R₅ comprises one or two acyl moieties, selected from fatty acid residues, hydroxy fatty acid residues and oxy fatty acid residues.

103. A process according to claim 93, wherein:
(a) R₅ is the 3-hydroxy C₁₄-alkanoyl ester-linked at the 3-hydroxy position with the C₁₂-fatty acid, and the acyloxyacyl group is at the N2′-position,
(b) R₅ is the 3-hydroxy C₁₄-alkanoyl ester-linked at the 3-hydroxy position with the C₁₄-fatty acid, and the acyloxyacyl group is preferably at the N-2′ position,
(c) R₅ is the 3-hydroxy C₁₄-alkanoyl ester-linked at the 3-hydroxy position with the C₁₂-fatty acid, with this acyloxyacyl group is at the N-2 position,
(d) R₅ is the 3-hydroxy C₁₄-alkanoyl ester-linked at the 3-hydroxy position with the C₁₂-fatty acid, with the acyloxyacyl group at both the N2-position and the N-2′-position,
(e) R₅ is 3-hydroxy (C₄-C₂₄)-fatty acid, preferably a 3-hydroxy (C₁₀-C₁₈)-fatty acid, most preferably 3-hydroxy C₁₄-fatty acid, at the N2-position or at the N2′-position which optionally may be protected,
(f) R₅ is 3-hydroxy (C₄-C₂₄)-fatty acid-acyl which is ester-linked at the 3-hydroxy position with (C₁-C₂₀)-carboxylic acid, preferably an 3-hydroxy (C₃-C₁₈)-fatty acid-acyl ester-linked at the 3-hydroxy position with (C₁₀-C₁₈)-fatty acid,
(g) R₅ at the N2 position is the 3-hydroxy C₁₄-alkanoyl ester-linked at the 3-hydroxy position with the C₁₂-fatty acid or C₁₆-fatty acid, and wherein R₅ at the N2′ position is the 3-hydroxy C₁₄-alkanoyl ester-linked at the 3-hydroxy position with the C₁₂-fatty acid or C₁₄-fatty acid,
(h) a first group R₅ is selected from the subgroup (i) as defined in claim 1 and a second group R₅ is selected from a subgroup (ii) or (iii) as defined in claim 1, wherein preferably the group R₅ at the N-2 position is selected from (i),
(i) two groups of R₅ are both selected identically or differently from the subgroup (i) or are both selected identically or differently from a subgroup (ii) or (iii),
(j) in the group R₅ the acyl groups and/or the acyl and alkyl group may be interlinked

104. A process according to claim 93 wherein in step (3) at least one of R₅ is selected from a branched acyl group as defined in (ii), (iii).

105. A process according to claim 104 wherein the group R₅ connected to the N-2′-position is selected as a branched acyl group.

106. A process according to claim 93 wherein step (3) is preferably carried out in the presence of a coupling agent.

107. A process according to claim 106 wherein the coupling agent is isobutyl chloroformate or 1-isobutyloxy 2-isobutyloxycarbonyl-1,2-dihydroquinoleine or a carbodiimide.

108. A process according to claim 93, wherein step (5) phosphorylation is carried out with tetrabenzyl pyrophosphate in the presence of a suitable base and a polar solvent.

109. A process according to claim 108 wherein the base is lithium bis(trimethylsilyl)amide and the polar solvent is THF.

110. A process according to claim 93, wherein step (6) is carried out by reaction with a sulfur trioxide complex in a solvent.

111. A process according to claim 110 wherein the sulfur trioxide complex is trimethyl amine sulfur trioxide complex and the solvent is DMF.

112. A process according to claim 93 wherein the step (7) is carried out in presence of a coupling agent such as isobutyl chloroformate or 1-isobutyloxy 2-isobutyloxycarbonyl-1,2-dihydroquinoleine or a carbodiimide.

113. A process according to claim 93 wherein the step (8) coupling reaction is carried out by reacting compound 14 with trichloroacetonitrile in the presence of a mineral base and a polar solvent.

114. A process according to claim 113 wherein the mineral base is cesium carbonate or potassium carbonate and the polar solvent is preferably an aprotic polar solvent such as dichloromethane.

115. A process according to claim 93, wherein the step (9) is carried out in a polar solvent.

116. A process according to claim 115 wherein the polar solvent is an aprotic polar solvent.

117. A process according to claim 116, wherein the solvent is dichloromethane.

118. A process according to claim 93, wherein the step (9) is carried out in the presence of catalytic amount of Lewis acid.

119. A process according to claim 118 wherein the Lewis acid is trimethylsilyltrifluoromethanesulfonate.

120. A process according to claim 93, wherein in the step (9) R₈ is in α or β configuration.

121. A process according to claim 93, wherein in step (11) protecting groups are removed by of hydrogenolysis.

122. A process according to claim 121, wherein hydrogenolysis is carried out in the presence of a high-grade metal.

123. A process according to claim 122 wherein the high-grade metal is such as palladium on carbon.
